(19) United States
(12) Patent Application Publication
(10) Pub. No.: US 2025/0352113 A1
(43) Pub. Date: Nov. 20, 2025

(54) Ablation Catheters with Deployable Electrode Structures for Use in Ablation and Electrophysiological Mapping

(71) Applicant: CARDIOFOCUS, INC., MARLBOROUGH, MA (US)

(72) Inventors: Gerald Melsky, Lexington, MA (US); Curtis Keohane, Brighton, MA (US); Omar Colon, Nashua, NH (US); Brian Connor, Bolton, MA (US); Christopher Brushett, Cambridge, MA (US); Jacob Laughner, Medford, MA (US)

(73) Assignee: CARDIOFOCUS, INC., MARLBOROUGH, MA (US)

(21) Appl. No.: 19/208,127

(22) Filed: May 14, 2025

Related U.S. Application Data
(60) Provisional application No. 63/647,274, filed on May 14, 2024.
(60) Provisional application No. 63/647,282, filed on May 14, 2024.

(51) Int. Cl. A61B 5/287; A61B 5/00; A61B 18/00

(52) U.S. Cl. CPC A61B 5/287; A61B 5/6853; A61B 5/6859; A61B 18/00; A61B 2018/0016; A61B 2018/00184; A61B 2018/00267; A61B 2018/00577; A61B 2018/0091; A61B 2018/00982; A61B 2560/0468

(57) ABSTRACT

A balloon catheter includes an inflatable balloon coupled to an outer catheter shaft and an axially translatable nose tip to which the inflatable balloon is coupled. An electrode basket surrounds the balloon and has a plurality of first splines and a plurality of second splines, wherein the plurality of first splines and the plurality of second splines are configured to deploy under inflation of the inflatable balloon and wherein at least some first splines of the plurality of first splines include at least one electrode. An actuator axially translates the nose tip in a longitudinal direction to facilitate the electrode basket moving to a collapsed state when the balloon is deflated and to an expanded state when the balloon is inflated. The actuator moves between an extended position and a retracted position and a lock mechanism locks the actuator in the extended position.

### CROSS REFERENCE TO RELATED APPLICATIONS

The present application claims the benefit of and priority to U.S. patent application Ser. No. 63/647,274, filed May 14, 2024, and U.S. patent application Ser. No. 63/647,282, filed May 14, 2024, each of which is hereby expressly incorporated by reference in its entirety.

### TECHNICAL FIELD

The present disclosure is directed to ablation of atrial fibrillation and specifically to ablation of atrial fibrillation with a device that includes a deployable structure to provide electrodes on the surface of an endoscopically guided laser ablation catheter for use in ablation and electrophysiological mapping.

### BACKGROUND

Balloon catheters that are configured to perform ablation of atrial fibrillation are well known and are described in Melsky et al U.S. Pat. No. 9,421,066B2 and Melsky et al U.S. Pat. No. 9,033,961B2, each of which is hereby expressly incorporated by reference in its entirety. The aforementioned patents treat atrial fibrillation by using an energy source to create non-electrically conducting lesions in the atrial tissue in such a fashion that a circumferential ring of lesion is created in the region of the left atrium where the pulmonary veins join the atrium. Such circumferential lesions prevent electrical signals originating in the veins from entering the atrium and vice versa. Blocking the passage of such electrical signals can, in most cases, restore sinus rhythm to a previously fibrillating left atrium.

Typically, ablation for atrial fibrillation consists of the steps of introducing an ablation catheter into the left atrium, creating the circumferential lesions around the pulmonary veins and then confirming that the circumferential lesions have been adequately produced so as to actually block electrical signals. This confirmation process generally consists of removing the ablation catheter then introducing a catheter with multiple electrodes which can be placed in a pulmonary vein distal to the circumferential lesion and then using the electrodes to monitor the electrograms originating in the pulmonary veins. When the vein has been electrically isolated from the atrium, the vein is silent with only far-field electrical activity seen in the vein. Occasional spikes within the vein may occur but with no conduction to the rest of the atrium. Pacing the atrium via a catheter with electrodes placed in the coronary sinus can help confirm that only far-field activity and random spikes are seen in the vein.

Now, the aforementioned devices in Melsky et al U.S. Pat. No. 9,421,066B2 and Melsky et al U.S. Pat. No. 9,033,961B2 are effective ablation devices but, as with many other ablation devices, they contain no means for quickly and easily confirming electrical isolation once the ablation of a vein has been completed. It is very desirable to be able to ablate veins and then, without having to exchange catheters, be able to confirm that the ablation has resulted in the desired electrical isolation of the veins. Therefore, one object of the present invention is to provide an ablation device that provides endoscopically guided laser ablation and provides a means to confirm that electrical isolation of the pulmonary veins has been achieved and to perform such confirmation without the need to remove or exchange catheters. Exchanging catheters carries the risk of introducing air into the left atrium if performed incorrectly. Air introduction into the left atrium could lead to damage to the brain or heart or of other organs should the air travel into the organs capillary beds and impede blood flow there. For this reason, catheter exchanges are always done slowly and methodically to minimize the risk of air introduction. However, slow and methodical catheter exchanges increase the time to complete an ablation procedure. Prolonged procedures carry other risks to the patient as well as increasing the cost of the procedure so reducing the number of catheter exchanges during a procedure is desirable.

In addition to confirming electrical isolation of the veins has been achieved, the addition of electrodes to the ablation catheters described in Melsky et al U.S. Pat. No. 9,421,066B2 and Melsky et al U.S. Pat. No. 9,033,961B2 would also enable the delivery of ablative energy that requires an electrically conductive pathway from the energy source to the region of ablation. The ablative energy delivered may be either radiofrequency energy or electroporative energy (also called pulsed field ablation energy) or other energy, such as laser or microwave. The ability to deliver these other ablative energy types may be desirable in instances where anatomical considerations favor one type of energy over the other. For example, laser energy is desirable because it creates lesions that penetrate through the full thickness of the atrial wall thus ensuring that the electrical disassociation caused by lesions created using laser energy will be robust and durable. However in circumstances where the esophagus lies against the left atrium in an area that must be ablated, use of electroporative energy in that particular region may be desirable since it has been proposed that electroporative energy creates lesions differentially in cardiac tissue and esophageal tissue thereby opening the possibility that cardiac tissue adjacent to the esophagus can be safely ablated via electroporation without the need to closely monitor the temperature of the esophagus and halt ablation if the esophagus temperature rises too high.

As mentioned, pulsed electric field therapy is one of the types of ablation therapies that has been developed due to its advantages over thermal ablation. In this technique, high voltage pulses of short duration voltage are applied to electrodes of a delivery catheter.

As is known, electroporation is a non-thermal ablation technique that involves applying strong electric-fields that induce pore formation in the cellular membrane. The electric field may be induced by applying a relatively short duration pulse. Such a pulse may be repeated to form a pulse train. When such an electric field is applied to tissue in an in vivo setting, the cells in the tissue are subjected to an increased trans-membrane potential, which opens the pores on the cell plasma membrane. Electroporation may be reversible (i.e., the temporally-opened pores will reseal) or irreversible (i.e., the pores do not reseal and will remain open). In certain targeted therapeutic applications, a suitably configured pulse train alone may be used to cause cell destruction, for instance by causing irreversible electroporation. For example, pulsed field ablation (PFA) may be used to perform instantaneous pulmonary vein isolation (PVI). PFA generally involves delivering high voltage pulses from electrodes disposed on a catheter. These fields may be applied between pairs of electrodes (bipolar therapy) or between one or more electrodes and a return patch (monopolar therapy).

In PFA, different waveforms may be used to achieve different goals. For example, some waveforms may result in larger or smaller lesion size than other waveforms. Further, some waveforms result in higher or lower overall energy delivery than other waveforms (less overall energy delivery generally corresponds to less heating of the target tissue). Thus, the waveform characteristics can be selected and customized in view of the specific given application.

### SUMMARY

In summary, one object of the present disclosure is to provide a means to quickly and easily confirm electrical isolation of pulmonary veins that have been isolated by endoscopically guided laser ablation using devices similar to those described in Melsky et al U.S. Pat. No. 9,421,066B2 and Melsky et al U.S. Pat. No. 9,033,961B2. A further object of the invention is to provide such means in such a manner that no catheter exchanges are required. A further object of the invention is to provide a means to both confirm isolation and to deliver other forms of ablative energy that can be delivered via electrodes which either contact the tissue or are in close proximity to tissue. A further object of the invention is to provide electrodes for either isolation confirmation or ablation that can be visualized endoscopically using the endoscopic apparatus already present in Melsky et al U.S. Pat. No. 9,421,066B2 and Melsky et al U.S. Pat. No. 9,033,961B2.

In one exemplary embodiment, an ablation balloon catheter includes:
a handle;
an outer catheter shaft coupled to the handle;
an inflatable balloon coupled at a first end to the outer catheter shaft;
an axially translatable nose tip to which a second end of the inflatable balloon is coupled;
an electrode basket surrounding the balloon and being coupled to the outer catheter shaft and to the nose tip, the electrode basket having a plurality of first splines and a plurality of second splines, wherein the plurality of first splines and the plurality of second splines are configured to deploy under inflation of the inflatable balloon and wherein at least some first splines of the plurality of first splines include at least one electrode;
an actuator disposed within the handle for axially translating the nose tip in a longitudinal direction to facilitate the electrode basket moving to a collapsed state when the balloon is deflated and to an expanded state when the balloon is inflated, the actuator moving axially within the handle between an extended position and a retracted position; and a lock mechanism for locking the actuator in the extended position.

### BRIEF DESCRIPTION OF DRAWING FIGURES

FIG. 1 shows one exemplary device of the present disclosure in a deployed state, deployed over a surface of an inflated balloon of an exemplary balloon catheter;

FIG. 2 shows the device of FIG. 1 in a retracted state ready to be advanced over a deflated balloon of the balloon catheter;

FIG. 3 shows the device of FIG. 1 in a state in which it has been advanced over an inflated balloon of a balloon catheter and it is in a partial state of deployment, such deployment being accomplished by inflation of the balloon;

FIG. 4 shows a PFA catheter mounted basket;

FIG. 5 shows an electrode catheter for use with a balloon catheter;

FIG. 6 shows a dual transeptal/second catheter device that includes the electrode catheter of FIG. 5 disposed over the balloon catheter;

FIG. 7 shows a retractable tine electrode array embodiment;

FIGS. 8A-8C show the various states of the retractable tine electrode array;

FIG. 9 shows a balloon catheter with PFA braided wire mesh electrode array;

FIG. 10 shows a balloon with embedded electrode array;

FIG. 11 shows balloon catheter with micropores with an inner electrode array;

FIG. 12 shows the balloon catheter with micropores with the inner electrode array;

FIG. 13 shows another balloon catheter with micropores and an inner electrode array;

FIG. 14 is a block diagram depicting exemplary components of an endoscope-guided cardiac ablation system according to the invention;

FIG. 15A is a perspective view of a translating tip balloon catheter according to one embodiment and being shown in a collapsed state;

FIG. 15B is a perspective view of the translating tip balloon catheter of FIG. 15A in an expanded state;

FIG. 16A is a perspective view of a handle suitable for use with the translating tip balloon catheter of FIG. 15A and being shown in an extended state;

FIG. 16B is a perspective view of the handle of FIG. 16A in a retracted state;

FIG. 17 is a top plan view of a catheter in accordance with one embodiment;

FIG. 18 is a close-up view of an expandable electrode basket that is at a distal end of a catheter shaft and is shown in an expanded state;

FIG. 19 is a close-up view of a distal end of a main shaft that is contained inside the expandable electrode basket;

FIG. 20 is a close-up view of an expandable electrode basket that is at a distal end of a catheter shaft and is shown in an expanded state;

FIG. 21 is a close-up view of a tip of the catheter;

FIG. 22 is a close-up view of a proximal end of the balloon and distal end of the shaft;

FIG. 23 is a cross-sectional of a section of the nitinol tube;

FIG. 24 is a close-up of a distal end of the main shaft and proximal end of the balloon;

FIG. 25 is a close-up of a section of the main shaft;

FIG. 26 is a close-up of a section of a handle;

FIG. 27 is a close-up perspective of a section of a handle according to another embodiment showing a first locking mechanism in the form of a knob ratcheting mechanism;

FIG. 28 is a cross-sectional view of the section of the FIG. 27;

FIG. 29 is a perspective view of a knob ratcheting mechanism showing a torsion spring thereof;

FIG. 30 is a cross-sectional view of the knob and an actuator shaft that is coupled to the knob at a distal end of the actuator shaft;

FIG. 31 is a perspective view showing the coupling between a torsion spring at a torsion cap that is coupled to the actuator shaft;

FIG. 32 is a close-up perspective view of a knob ratcheting mechanism showing meshing surfaces;

FIG. 33 is a close-up perspective view of an alternative knob lock mechanism;

FIGS. 34A-34C are close-up perspective views of another knob lock mechanism, with FIG. 34A showing an unlocked position; FIG. 34B showing an unlocked position with the actuation tube moved in a distal direction and FIG. 34C showing a locked position;

FIGS. 35A-35B are sectional views showing the knob lock mechanism of FIGS. 34A-34C in an unlocked position (FIG. 35A) and a locked position (FIG. 35B);

FIG. 36 is a close-up of the expandable electrode basket in the collapsed state;

FIG. 37 is a close-up of the expandable electrode basket in the expanded state;

FIG. 38A-38C illustrate different electrode marker patterns;

FIG. 39A is an image of a balloon catheter with electrode basket under fluoroscopy with spline (electrode) #1 being visually marked;

FIG. 39B is an image of a balloon catheter with electrode basket under fluoroscopy with spline (electrode) #1 being visually marked, as well as first and second reference markers on other splines;

FIG. 40 is a perspective view of a 3D mapping image showing in real-time the balloon catheter at the target surgical site;

FIG. 41A is a fluoroscopic image of a balloon catheter with radiopaque markers on two different splines that form the collapsible electrode basket;

FIG. 41B is an initial random endoscopic image of the balloon catheter in the position shown in FIG. 41A;

FIG. 41C is a final endoscopic image that has been manipulated (e.g., rotated) from FIG. 41B such that the top of the image is towards the patient's head;

FIG. 42A is a fluoroscopic image of a balloon catheter with radiopaque markers on two different splines that form the collapsible electrode basket;

FIG. 42B is an initial random endoscopic image of the balloon catheter in the position shown in FIG. 42A;

FIG. 42C is a final endoscopic image that has been manipulated (e.g., rotated) from FIG. 42B such that the top of the image is towards the patient's head;

FIG. 43A is a fluoroscopic image of a balloon catheter with radiopaque markers on two different splines that form the collapsible electrode basket;

FIG. 43B is an initial random endoscopic image of the balloon catheter in the position shown in FIG. 43A;

FIG. 43C is a final endoscopic image that has been manipulated (e.g., rotated) from FIG. 43B such that the top of the image is towards the patient's head;

FIG. 44A is a fluoroscopic image of a balloon catheter with radiopaque markers on two different splines that form the collapsible electrode basket;

FIG. 44B is an initial random endoscopic image of the balloon catheter in the position shown in FIG. 44A;

FIG. 44C is a final endoscopic image that has been manipulated (e.g., rotated) from FIG. 44B such that the top of the image is towards the patient's head;

FIG. 45A is a fluoroscopic image of a balloon catheter with radiopaque markers on two different splines that form the collapsible electrode basket;

FIG. 45B is an initial random endoscopic image of the balloon catheter in the position shown in FIG. 45A;

FIG. 45C is a final endoscopic image that has been manipulated (e.g., rotated) from FIG. 45B such that the top of the image is towards the patient's head; and

FIG. 46 is a system diagram that includes the catheter with an endoscopic, an image signal processing device, an image rotation processing device, and a display device.

### DETAILED DESCRIPTION OF CERTAIN EMBODIMENTS

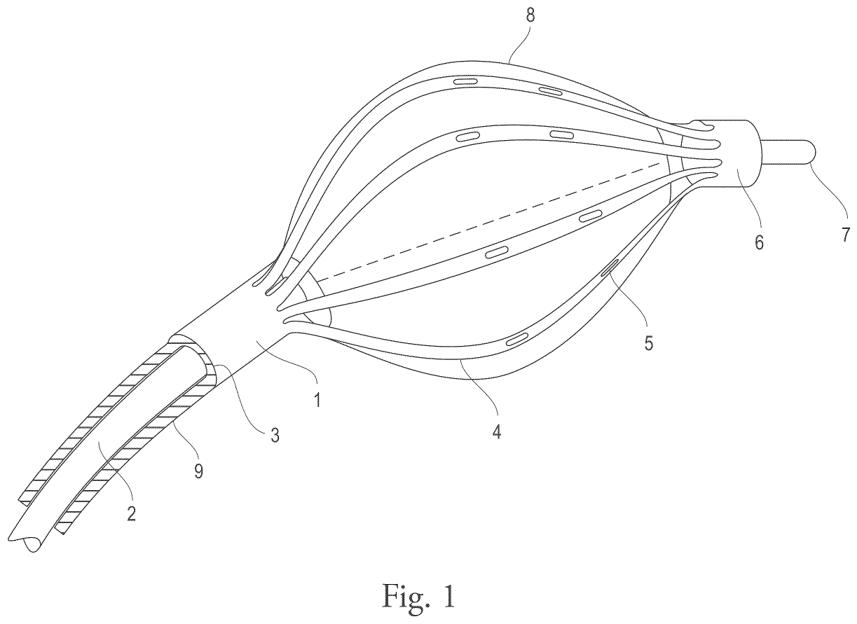

FIG. 1 shows an exemplary balloon catheter, such as the one described in Melsky et al U.S. Pat. No. 9,421,066B2 and Melsky et al U.S. Pat. No. 9,033,961B2, each of which has been incorporated by reference.

### Exemplary Ablation System

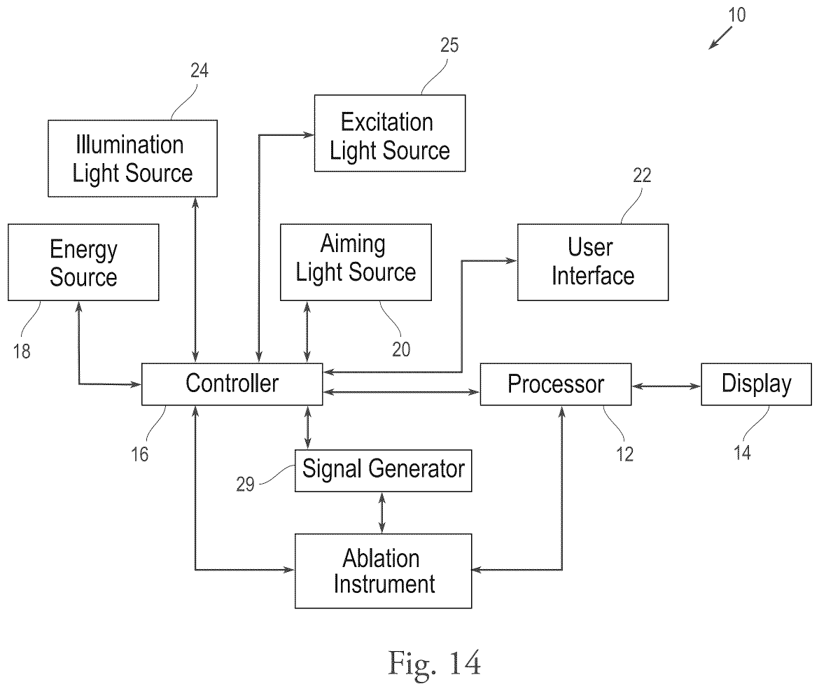

FIG. 14 is an exemplary schematic block diagram illustrating one ablation/endoscopic system in accordance with the invention, designated generally by reference numeral 10. Ablation system 10 preferably includes a treatment ablation instrument, such as one of the ones described herein, preferably including an endoscope and ablation device as discussed below.

The ablator system 10 further preferably includes an aiming light source 20 and an illumination light source 24. A processor 12 designed to accept input and output data from the connected instruments, a display 14, and a controller 16 and process that data into visual information.

As will also be appreciated from the discussion below, an endoscope is preferably provided in ablation instrument 100 and has the capability of capturing both live images and recording still images. An illumination light 24 is used to provide operating light to the treatment site. The illumination light is of a frequency that allows the user to differentiate between different tissues present at the operating site. An aiming light source 20 is used to visualize the location where energy will be delivered by the ablation instrument 100 to tissue. It is envisioned that the aiming light 20 will be of a wavelength that can be recorded by an image capture device and visible on a display.

The processor 12 can be designed to process live visual data as well as data from the ablation instrument controllers and display. The processor 12 is configured execute a series of software and/or hardware modules configured to interpret, manipulate and record visual information received from the treatment site. The processor 12 can further be configured to manipulate and provide illustrative and graphical overlays and composite or hybrid visual data to the display device.

As seen in FIG. 14, the system 10 further includes the controller 16, an energy source 18, the aiming light source 20 and a user interface 22. Controller 16 is preferably configured to control the output of the energy source 18 and the illumination and excitation sources 24 and 25 of an energy transmitter, as well as being configured to determine the distance and movement of an energy transmitter relative to tissue at an ablation treatment site (as discussed further below). As will also be appreciated from the below discussion, an endoscope is preferably supported by the ablation instrument and captures images that can be processed by the processor 12 to determine whether sufficient ablative energy deliveries have been directed to a specific area of a treatment site. Data obtained from the endoscope includes real-time video or still images of the treatment site as seen from the ablation instrument. As discussed herein, these images/videos can be stored in memory for later use.

The aiming light source 20 is used to visualize the treatment site location where energy will be delivered by the ablation instrument to the target tissue. Preferably, the aiming light source outputs light in a visible region of the electromagnetic spectrum. If a suitable ablation path is seen by the user, the controller 16 can transmit radiant energy, via energy source 18, from the ablation instrument to a target tissue site to effect ablation by lesions. It is to be appreciated that the term “radiant energy” as used herein is intended to encompass energy sources that do not rely primarily on conductive or convective heat transfer. Such sources include, but are not limited to, acoustic, laser, electroporative energy, and electromagnetic radiation sources and, more specifically, include microwave, x-ray, gamma-ray, ultrasonic and radiant light sources. Additionally, the term “light” as used herein is intended to encompass electromagnetic radiation including, but not limited to, visible light, infrared and ultraviolet radiation.

The illumination light source 24 is a light source used to provide proper illumination to the treatment site. The illumination is configured so that natural biological tones and hues can be easily identifiable by an operator.

The controller 16 can provide the user with the ability to control the function of the aiming light source, the user input devices, and the ablation instrument. The controller 16 serves as the primary control interface for the ablation system. Through the controller 16, the user can turn on and off both the aiming and illumination lights 20, 24. Furthermore the controller 16 possesses the ability to change the illumination and aiming light intensity. The ability to switch user interfaces or display devices is also envisioned. Additionally, the controller 16 gives access to the ablation instrument, including control over the intensity of the discharge, duration and location of ablative energy discharges. The controller 16 can further provide a safety shutoff to the system in the event that a clear transmission pathway between the radiant energy source and the target tissue is lost during energy delivery (e.g., see commonly owned U.S. patent application Ser. No. 12/896,010, filed Oct. 1, 2010, which is hereby incorporated by reference in its entirety).

The controller can be a separate microprocessor based control interface hardware or it can be a portion of a configured as a module operating through a processor based computer system configured to accept and control inputs from various physical devices.

### Pulsed Electric Field Ablative Energy

While the technical field of pulsed electric fields for tissue therapeutics continues to evolve, it is generally understood that application of brief high DC voltages to tissue may generate locally high electric fields typically in the range of hundreds of volts per centimeter that disrupt cell membranes by generating pores in the cell membrane. While the precise mechanism of this electrically-driven pore generation or electroporation continues to be studied, it is thought that the application of relatively brief and large electric fields generates instabilities in the lipid bilayers in cell membranes, causing the occurrence of a distribution of local gaps or pores in the cell membrane. This electroporation may be irreversible if the applied electric field at the membrane is larger than a threshold value such that the pores do not close and remain open, thereby permitting exchange of biomolecular material across the membrane leading to necrosis and/or apoptosis (cell death). Subsequently, the surrounding tissue may heal naturally.

Generally, a system, such as the ones described herein, for delivering a pulse waveform to tissue includes a signal generator configured for generating a pulse waveform and an ablation device coupled to the signal generator and configured to receive the pulse waveform. In some embodiments, the ablation device is configured to generate an electric field intensity of between about 200 V/cm and about 1500 V/cm. Accordingly, a system for ablating tissue described herein can include a signal generator and an ablation device having one or more electrodes and an expandable/inflatable member (e.g., balloon) for the selective and rapid application of DC voltage to drive electroporation.

In some embodiments, the voltage pulse waveforms disclosed herein may be hierarchical and have a nested structure.

An irreversible electroporation system as described herein may include a signal generator and a processor configured to apply one or more voltage pulse waveforms to a set of electrodes to deliver energy to a region of interest. In order to deliver the pulse waveforms generated by the signal generator, one or more electrodes of the ablation device may have an insulated electrical lead configured for sustaining a voltage potential of at least about 2500 V without dielectric breakdown of its corresponding insulation at least in one embodiment. In some embodiments, at least some of the electrodes may be independently addressable such that each electrode may be controlled (e.g., deliver energy) independently of any other electrode of the device.

As shown in FIG. 14, the system can include a signal generator 29 that is configured to generate pulse waveforms for irreversible electroporation of tissue, such as, for example, a pulmonary vein. For example, the signal generator 29 can be a voltage pulse waveform generator and be configured to deliver a pulse waveform to one of the ablation devices (ablation instruments) described herein. The processor 12 can incorporate data received from memory to determine the parameters of the pulse waveform to be generated by the signal generator 29, while some parameters such as voltage can be input by a user. The memory can further store instructions to cause the signal generator 29 to execute modules, processes and/or functions associated with the system, such as pulse waveform. For example, the memory can be configured to store pulse waveform for pulse waveform generation.

Some embodiments are directed to pulsed high voltage waveforms together with a sequenced delivery scheme for delivering energy to tissue via sets of electrodes. The signal generator and the processor are capable of being configured to apply pulsed voltage waveforms to a selected plurality or a subset of electrodes of an ablation device.

In one application, a pulsed voltage waveform can be in the form of a sequence of double pulses, with each pulse, such as the pulse being associated with a pulse width or duration. The pulse width/duration can be about 0.5 microseconds, about 1 microsecond, about 5 microseconds, about 10 microseconds, about 25 microseconds, about 50 microseconds, about 100 microseconds, about 125 microseconds, about 140 microseconds, about 150 microseconds, including all values and sub-ranges in between. The pulsed waveform can be defined by a set of monophasic pulses where the polarities of all the pulses are the same (e.g., all positive, as measured from a zero baseline). In some embodiments, such as for irreversible electroporation applications, the height of each pulse or the voltage amplitude of the pulse can be in the range from about 400 volts, about 1,000 volts, about 5,000 volts, about 10,000 volts, about 15,000 volts (e.g., in one application a maximum amplitude of 2500 volts is used), including all values and sub ranges in between. The pulse is separated from a neighboring pulse by a time interval, also sometimes referred to as a first-time interval. As examples, the first time interval can be about 1 microsecond, about 50 microseconds, about 100 microseconds, about 200 microseconds, about 500 microseconds, about 800 microseconds, about 1 millisecond including all values and sub ranges in between, in order to generate irreversible electroporation. It will be appreciated that the aforementioned values are only exemplary in nature and are not limiting of the scope of the present invention since values outside the aforementioned ranges can exist for other applications.

### Exemplary Ablation Catheter

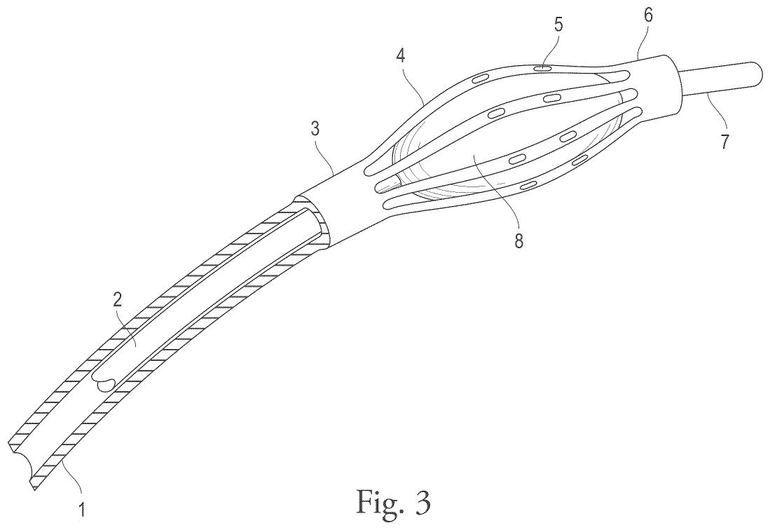

As shown in FIGS. 1-3, one exemplary ablation device is directed to a generally flexible and elongate structure 1, that is slidably disposed over an elongate shaft 2 of a balloon ablation catheter. The elongate structure 1 can be considered to be a sleeve that is longitudinally displaceable over the balloon catheter. While the term “elongate structure” is used herein, it will be understood that the term “sleeve” can be interchangeably used therewith. As described herein, the elongate structure 1 can be moved along the balloon catheter so as to cover different regions of the balloon catheter. As described herein, the elongate structure 1 is configured to respond to the movements of the balloon ablation catheter and more particularly, to the expansion and contraction of the balloon when the elongated structure at least partially covers the balloon.

The elongate structure 1 generally has several different portions including a proximal portion and a distal portion. The proximal portion of the elongate structure 1 comprises a first tubular shaped portion 3 as shown in FIG. 1. This proximal region is spaced back from the distal end at a distance of 2 cm to 4 cm; however, this is merely one exemplary value and not limiting of the scope of the present invention. The first tubular shaped portion 3 is configured such that the shaft 2 of the balloon ablation catheter passes through a lumen of the first tubular shaped portion 3. In other words, the first tubular shaped portion 3 completely surrounds the catheter shaft 2 in at least one region of the first tubular shaped portion 3.

The first tubular shaped portion 3 can be formed of a flexible material.

The distal portion of the elongated structure 1 multifurcates into two or more but preferably six or more branches 4, which are also flexible. Each branch 4 contains one or more electrodes 5 on their outward facing surface. Each electrode 5 is connected to an insulated conductor wire embedded in the body of the elongated flexible structure 1 but such conductor wires or the like are not shown in FIG. 1. For example, the structure 1 can be overmolded over the conductor wires. As shown, when multiple electrodes 5 are used for each branch 4, the electrodes 5 are spaced longitudinally apart along the respective branch 4. It will also be appreciated that the electrodes 5 can be of the same type or can be of different types. In other words, the electrodes 5 can be of different sizes and/or different shapes. The arrangement of the electrodes 5 can be of an asymmetric nature in that the electrodes 5 can be focused on one or more regions of the branches 4. For example, the electrodes 5 can be more centrally located and distally located along the branches 4 as opposed to be located proximally.

The branches 4 can thus be circumferentially spaced apart from one another and extend circumferentially about the balloon. It is also possible for the branches 4 to be designed to have an asymmetric appearance in that instead of having a symmetric angular displacement between the branches 4, an asymmetric arrangement can be provided. In other words, within one half of the elongated structure 1, the branches 4 can have one type of angular displacement and within the other half, a different angular displacement can be provided. In other words, there can be more branches 4 in one half of the structure 1 compared to the other half of the structure 1. For example, the first circumferential half can have a first number of electrodes, while the second circumferential half can have a second number of electrodes that can be different than the first number.

As shown, each branch 4 has a first end (proximal end) and an opposing second end (distal end). The first ends of the branches 4 are attached to the first tubular shaped portion 3 and in one embodiment, the branches 4 are formed integral with the first tubular shaped portion 3.

The multiple flexible branches 4 rejoin at their second ends to again form a second tubular structure 6 at the distal end of the elongate structure 1. The second tubular structure 6 encircles, in a slidable manner (both axially and rotationally), a distal tip 7 of the balloon ablation catheter.

In general, the multifurcations (branches 4) form an expandable cage like structure which circumferentially surrounds the inflated balloon 8 when the elongated structure is positioned over at least a portion of the balloon. The proximal portion of the elongate structure 1 can maintain a tubular shape proximally from the multifurcations (branches 4) on back or, alternatively, the proximal portion of the elongate structure 1 can consist of only a partial circumferential portion of a tube as shown at 9 and thereby be more flexible and occupy less volume than if it were entirely tubular. Shaft 2 can be visible between portions of the elongated structure 1.

It will be appreciated that the present device 1 is preferably formed as a single elongate structure in which the tubular portions 3, 6 and branches 4 located therebetween are formed as a single unitary part (e.g., molded part).

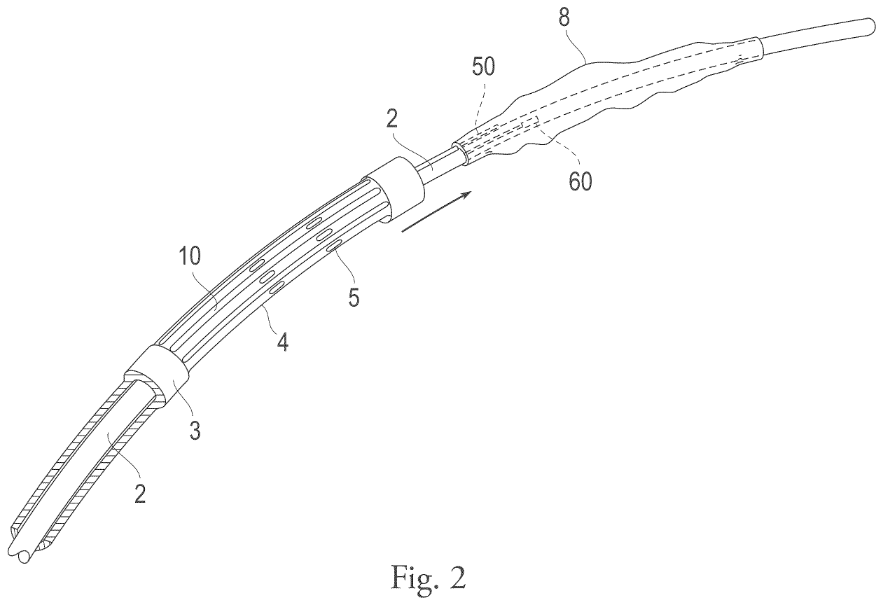

FIG. 2 shows travel of the elongated structure 1 over the balloon catheter. More particularly, the first tubular shaped portion 3 and the branches 4 are shown in their relaxed state. This represents the normal, at rest state of the elongate structure 1. In this state it is clear to see how such a structure can be produced by creating a series of longitudinal slits 10 in a generally thin flat material that has been formed into a tubular shape. In other words, the branches 4 are formed by incorporating longitudinal slits in the structure 1 so as to define one branch between two adjacent slits. A suitable thin flat material would be polyimide film such as is commonly used to produce flexible printed circuits or flex-circuits. It will be appreciated that other materials are equally possible.

FIGS. 1 and 2 together illustrate how the present device accomplishes the objectives of providing a means to allow for pulmonary vein isolation using an endoscopically guided balloon catheter and to additionally provide a means to confirm electrical isolation of the vein without the need to exchange catheters as required in the prior art. As discussed in more detail below, the inner surface of the tubular structure can contain marks on the inside surface that are visible to the endoscope for indicating the location of the electrodes.

There can thus be two defined stages of operation including a first stage which is an ablation stage of the procedure in which the elongate structure 1 is not used. During this ablation stage, the elongate structure 1 resides, as shown in FIG. 2, proximal to the balloon of the balloon catheter and in a collapsed state closely surrounding the shaft 2 of the balloon catheter. As shown in this stage and state, the entire elongate structure 1 is displaced from and located proximal to the balloon of the balloon catheter. The distal second tubular shaft portion 6 is thus located proximal to the balloon.

In this state (first stage), the present elongate structure 1 allows for the balloon of the ablation catheter to be inflated and placed in a pulmonary vein while not being encumbered by the elongate structure 1. The vein may be visualized endoscopically by the ablation catheter and laser energy may be delivered to the vein without regard to the invention. In other words, as in Applicant's previous ablation catheter designs, energy from a movable energy emitter 0 (FIG. 2) that resides within the balloon passes through the balloon to the target site without any impediment from the elongate structure 1 due to the elongate structure 1 being spaced from and not in contact with the inflated operating area of the balloon.

This would not be the case if electrodes (such as electrodes 5) had been placed directly on the surface of the balloon since such electrodes would block both the laser energy and endoscopic visualization over the portion of the balloon on which such electrodes resided.

Once ablation (the first stage) of a vein has been accomplished, the balloon of the ablation catheter is deflated but the elongate structure 1 of the ablation catheter is not repositioned relative to the vein. With the ablation catheter structure stationary relative to the ablated vein, the elongate structure 1 is advanced distally over the deflated balloon. The balloon is then re-inflated and such re-inflation expands the branches (multifurcations) 4 of the elongate structure 1 and forces at least some number of the electrodes 5 into contact with the lumen of the vein. Said electrodes 5 can now be used to confirm electrical isolation by connecting the conductor wires connected to the electrodes 5 and extending proximally along the proximal portion of the elongate structure 1 until they are present outside of the patient's body, to know devices which are capable of amplifying and displaying the electrical activity emanating from the tissue in contact with the electrodes 5.

It should also be noted that the electrodes 5, when in this state of contact with the pulmonary vein tissue (or other target tissue) are also capable of delivering ablative energy such as radiofrequency energy or electroporative energy or microwave energy by connecting a source of such energy to the conductor wire attached to the electrodes. It should be also noted that the positions of the electrodes are visible to the endoscope 50 (FIG. 2) which resides inside the balloon of the ablation catheter. This visibility is accomplished by either making the multifurcations 4 out of a transparent material or by creating marks on the inner surfaces of the multifurcations directly adjacent to the position of the electrodes. Such visualization of the electrode position endoscopically enables a visual assessment of the condition of contact between electrodes and tissue. For example, a given electrode may be in firm contact with the vein tissue throughout the entire cardiac cycle. Alternatively, the electrode 5 can be in contact with tissue during a portion of the cardiac cycle and during the other portion of the cycle, the electrode 5 may not be in contact with tissue but it is in contact with blood instead or the electrode may not be in contact with tissue during any part of the cardiac cycle. Such visual assessments of the nature of the contact between tissue and the electrodes in not currently available in any known devices. Such assessment is valuable in aiding interpretation of the electrograms measured by the electrodes. Further, if the electrodes are to be used for the purposes of applying radiofrequency or electroporative or microwave ablation energy, such visual information about the degree of tissue contact can be used to determine which of the several electrodes are suitable to deliver ablative energy by virtue of the degree of tissue contact they afford. Also, the endoscopic view can be used to guide the repositioning of the balloon in the vein in order to improve the contact between electrodes and the vein tissue if deemed necessary for a better assessment of the electrical activity in the vein or for better electrode contact to enable ablation via radiofrequency or electroporative energy application.

### Sliding Action of Elongate Structure 1

As discussed herein, the elongate structure 1 is configured to move longitudinally along the balloon catheter as illustrated in FIGS. 1-3. It is also to have rotational movement relative to the balloon. The elongate structure 1 can be moved manually as by grasping one end (such as the first tubular portion 3) of the elongate structure 1 and the moving the entire structure 1 longitudinally in a distal or proximal direction. Alternatively, to move the elongate structure 1 in the proximal direction, the first tubular portion 3 can be grasped and pulled in the proximal direction. Preferably, the first tubular portion 3 extend proximally to a point where it exits the body and is available to be grasped directly by the user. To assist the user in moving the structure 1, the most proximal end of the structure 1 can have a grasp feature, such as an enlarged ring section or the like at the proximal end of the first tubular portion 3. Alternatively, surface texture or the like can be provided to one or more regions of the first tubular portion 3.

When the elongate structure 1 is retracted and moved proximally, it can enter into a lumen formed in the catheter structure or into a lumen in a guiding sheath or deflectable sheath commonly employed in atrial ablation procedures, through which the balloon catheter and tubular structure would be passed. This to say that the tubular structure can be slid so that it is retracted into the catheter shaft or into a guiding or deflectable sheath and this retraction will cause the elongate structure 1 to collapse and be removed from surrounding relationship around the balloon. The retraction of the structure 1 within the lumen of the catheter shaft causes the collapsing of branches to a compact state. It is noted that when the tubular structure is retracted into a guiding or deflectable sheath the multifurcations of the tubular structure are supported and prevented from expanding or deflecting outwardly by the inner surface of such sheath and are also prevented from deflecting inwardly by the shaft of the balloon catheter. In such a state the tubular structure is constrained from expanding or contracting and is therefore more easily repositioned relative to the balloon catheter. In the case of a device were the only ablative energy employed is delivered via the electrodes, the elongate structure would not necessarily need to be retracted to a position fully proximal of the balloon. In other words, the elongate structure 1 is movable between a multitude of positions with one position being a position in which at least some electrodes at least partially cover the balloon.

### Controllable Electrodes

The overall ablation system described herein that includes the elongate structure 1 and the ablation balloon catheter can communicate over a network to the various machines that are configured to send and receive content, data, as well as instructions that, when executed, enable operation of the various connected components/mechanisms. The content and data can include information in a variety of forms, including, as non-limiting examples, text, audio, images, and video, and can include embedded information such as links to other resources on the network, metadata, and/or machine executable instructions. Each computing device can be of conventional construction, and while discussion is made in regard to servers that provide different content and services to other devices, such as mobile computing devices, one or more of the server computing devices can comprise the same machine or can be spread across several machines in large scale implementations, as understood by persons having ordinary skill in the art. In relevant part, each computer server has one or more processors, a computer-readable memory that stores code that configures the processor to perform at least one function, and a communication port for connecting to the network. The code can comprise one or more programs, libraries, functions or routines which, for purposes of this specification, can be described in terms of a plurality of modules, residing in a representative code/instructions storage, that implement different parts of the process described herein. As described herein, each of the robotic devices (tools) has a controller (processor) and thus, comprises one form of the above-described computing device.

Further, computer programs (also referred to herein, generally, as computer control logic or computer readable program code), such as imaging or measurement software, can be stored in a main and/or secondary memory and implemented by one or more processors (controllers, or the like) to cause the one or more processors to perform the functions of the invention as described herein. In this document, the terms “memory,” “machine readable medium,” “computer program medium” and “computer usable medium” are used to generally refer to media such as a random access memory (RAM); a read only memory (ROM); a removable storage unit (e.g., a magnetic or optical disc, flash memory device, or the like); a hard disk; or the like. It should be understood that, for mobile computing devices (e.g., tablet), computer programs such as imaging software can be in the form of an app executed on the mobile computing device.

The system can include a graphical user interface (GUI) that can be provided to allow for remote control over the system. As is known, the GUI is a system of interactive visual components for computer software. A GUI displays objects that convey information and represent actions that can be taken by the user. The objects change color, size, or visibility when the user interacts with them. GUI objects include icons, cursors, and buttons. These graphical elements are sometimes enhanced with sounds, or visual effects like transparency and drop shadows.

The graphical user interface typically includes a display, such as a touch screen display to allow user input to be registered and then steps are taken by the main controller (main processor).

In one exemplary embodiment, the main controller can be used to control the operation of the electrodes 5. In other words, select electrodes 5 can be operated (activated) at a given time using the main controller. Those electrodes 5 that are activated are supplied with ablative energy, while those electrodes 5 that are not activated are not supplied with ablative energy. As mentioned, the electrodes 5 can be wired to an electrical connector that is itself connected to a terminal (console) or the like (e.g., an outlet or plug thereof), thereby providing power to the electrodes 5.

Depending upon certain parameters, such as the location of the balloon catheter in the body, certain electrodes 5 can be activated and turned on, while certain electrodes 5 can be turned off and not activated. For example, if the balloon catheter and tubular structure are contacting certain tissue and such contact with tissue is being visualized by an endoscope inside the balloon, the user may want only those electrodes 5 that are in contact with tissue to receive ablative energy and therefore, based on endoscopic guidance or the like, the operator can strategically select which branches 4 and electrodes 5 that are to be activated.

The master controller can communicate with a display, such as display 14, on which images and data can be displayed.

A touch screen or the like can be used to select the branches 4 and electrodes 5 that are to be activated (energized). For example, a graphic image of the elongated structure 1 and more specifically, a graphic image of the branches 4 and the electrodes 5, can be displayed to the operator and then the operator can select those branches 4/electrodes 5 to be activated. When a touch screen is used, the operator can simply highlight and select with a finger those branches 4/electrodes 5 that are to be activated. It will also be appreciated that AI based software can be used to determine and then recommend to the user which electrodes should be activated based on the determination that those electrodes are in contact with tissue.

### PFA Catheter Mounted Basket

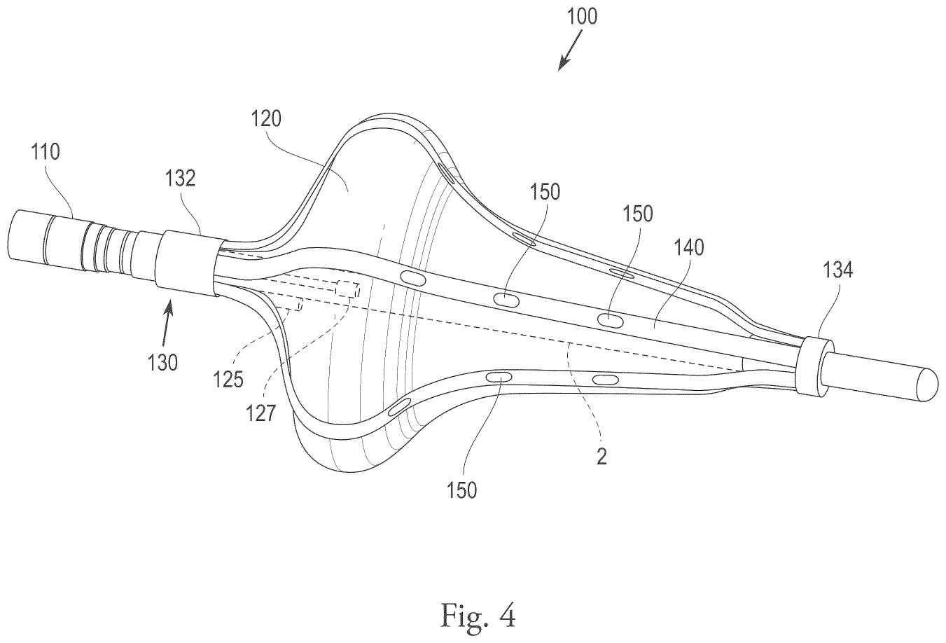

FIG. 4 illustrates a balloon catheter 100 that includes a main catheter shaft 110 that has a distal end. It will also be appreciated that the balloon catheter 100 typically includes more than one shaft and often includes an inner catheter shaft and an outer catheter shaft or can otherwise include multiple concentric tubular structures. An inflatable balloon 120 is included and is coupled to the main catheter shaft 110 with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

FIG. 4 also shows an inner shaft 115 along with an endoscope 125. The endoscope 125 extends along the exterior of the inner shaft 115 and is typically located at one end of the balloon is forward looking in that it looks forward toward the other end of the balloon.

The inflatable balloon 120 is preferably a compliant balloon.

The inflatable balloon 120 also includes an endoscope 125 that is located within the compliant balloon. The endoscope allows the operator of the catheter to visualize the balloon surface and thereby aim the laser energy to those portions of the balloon surface which contact the atrial tissue it is desired to treat with the laser energy. Such a system is described in Melsky et al. (U.S. Pat. No. 9,421,066 (the '066 patent) and Melsky et al. (U.S. Pat. No. 9,033,961 (the '961 patent), each of which is incorporated by reference in its entirety. The endoscope is at a location that is proximal to the location at which the energy is delivered to the tissue to allow the user to view the delivery of the energy and the resultant tissue lesion(s). The endoscope can be one of the ones described herein and also one that is described in any one of the documents incorporated by reference herein.

In FIG. 4, an energy emitter 127 is illustrated; however, it will be appreciated that in the embodiment in which the electrode array is intended to remain in a position surrounding the inflatable balloon 120, then the energy emitter 127 can be eliminated or is present but never used. In the event that the electrode array can be displaced off of the balloon, then the energy emitter 127 can be used.

The endoscope 125 is forward-facing and is disposed adjacent to one of the catheter shafts, such as a central tubing typically formed of a transparent polymer material. As used herein, the term forward-facing refers to the view of the endoscope in a distal direction relative to the catheter body. Similarly, the term side-facing refers to the view of the endoscope in a direction that is radially outward from a side of the catheter body.

The endoscope 125 can be a fiber optic endoscope that is inserted through a lumen of the catheter and located within a proximal region of the inflatable balloon 120.

In another embodiment, the ablation catheter 100 includes first and second imaging devices for providing direct visualization of the region to be treated, with the first imaging device being fixed relative to the catheter body. The first and second imaging devices can be in the form of first and second imaging chip endoscopes. Details of the first and second imaging chip endoscopes are described in U.S. patent application Ser. No. 17/524,472, which is expressly incorporated herein by reference in its entirety.

The balloon catheter 100 includes an expandable basket 130 that surrounds the inflatable balloon 120 and is configured to expand upon expansion (inflation) of the inflatable balloon 120 and similarly, is configured to contract upon deflation and contraction of the inflatable balloon 120. The expandable basket 130 has a first collar (first ring) 132 at a first (proximal) end of the expandable basket 130 and a second collar (second ring) 134 at a second (distal) end of the expandable basket 130. The first and second collars 132, 134 have annular shapes and can thus have a continuous ring shape. The size of the two collars 132, 134 can be different from one another with the first collar 132 in the illustrated embodiment being larger than the second collar 134. The two collars 132, 134 are sized and configured to fixedly couple the expandable basket 130 to the main catheter shaft 110 (or one or more other catheter shafts) with the inflatable balloon 120 being located between the two collars 132, 134. The first collar 132 is thus preferably located proximal to the inflatable balloon 120, while the second collar 134 is located distal to the inflatable balloon 120.

The expandable basket 130 includes a plurality of splines 140 that are attached at one end to the first collar 132 and at the other end to the second collar 134. The plurality of splines 140 extend longitudinally along a length of the inflatable balloon 120. The plurality of splines 140 are circumferentially offset from one another with open spaces formed between adjacent splines 140. The splines 140 are constructed to expand and contract under action of the underlying inflatable balloon 120. In particular, when the inflatable balloon 120 expands under inflation, the splines 140 expand outwardly and conversely, when the inflatable balloon 120 contracts under deflation, the splines 140 contract inwardly.

The splines 140 thus conform to the shape of the inflatable balloon 120.

Each spline 140 carries one or more electrodes 150. For example, each spline 140 can include a plurality of electrodes 150 that can be described as being an electrode array. In the illustrated embodiment, there are three electrodes 150 located along the length of the spline 140. The electrodes 150 are spaced longitudinally along the spline (in series). The electrodes 150 are thus spaced apart from one another a predefined set distance. The locations of the splines 140 along the spline 140 are selected so as to centrally position the electrodes 150 relative to the inflatable balloon 120 since when the inflatable balloon 120 is inflated, the electrodes 150 are, as discussed herein, for placement against the target tissue to be ablated using PFA technique.

The electrodes 150 that define the electrode can be the same electrode type or they can be different. For example, the shapes and sizes of the electrodes 150 can be the same as shown. The material of the expandable basket 130 is not elastic in that the splines do not stretch elastically in a longitudinal direction but can expand and contract with the underlying inflatable balloon 120. Thus, the longitudinal spacing between the electrodes 150 does not change when the expandable basket 130 moves between the expanded position and the retracted position. Instead, it is a fixed distance which is important and this information is used during the visualization and ablation process in order to form the desired lesion as discussed herein.

Compared to the embodiment of FIGS. 1-3, FIG. 4 shows a product in which the expandable basket 130 is fixed at least in one embodiment.

In yet another aspect of the present disclosure, the system can include electrode markers which mark the location of the electrodes 150 along the splines. In particular, the electrodes 150 are located on the outer surface of the splines 140 and the splines are typically formed of a non-transmissive material and therefore, the electrodes 150 are not visible in the live endoscope image. Since the splines 140 are typically formed of an opaque material, the electrodes 150 cannot be seen since the endoscope 125 only sees the inner surface of the splines 140. In order for the locations of the electrodes 150 to be determined during visualization (i.e., use of the endoscope 125), markers (markings that can be seen during visualization) can be provided along the inner surface and/or outer surface of the spline 140. Each marker is located on the inner surface of the spline 140 directly opposite the location of the electrode 150 to mark the location of the electrode 150. The markers are visually identifiable in the live endoscopic image and therefore can be in the form of visual indicia formed along the inner surface of the spline 140. For example, the visual indicia can be in the form of numbers and/or text indicia. In addition, the visual indicia is selected such that one electrode can be differentiated from another electrode. For example, each spline can be numbered, such as spline 1, and then each electrode 150 can be lettered, such as A, B, C, etc. Thus, in the illustrated embodiment, the most distal electrode of spline 1 can be identified by marker 1A, the middle electrode can be identified by marker 1B and the most proximal electrode can be identified by marker 1C. Similarly, for the adjacent spline 2, the markers can be 2A, 2B, and 2C. It will be appreciated that there are many different ways to visually identify one electrode on one spline from another electrode on another spline.

For example, color can be used to identify one spline 140 from the other ones. For example, the letters A, B, C or numbers 1, 2 and 3 can be in one color for one spline and another color for another spline. Symbols can also be used as the markers.

It will be appreciated that not all of the electrodes 150 are visible in the live endoscopic image since not all of the electrodes are in the desired contact with tissue at the target site and therefore it is important to understand which electrodes are visible in the live endoscopic image and in contact with tissue so that these electrodes can be actuated (activated).

The movement of the expandable basket 130 and the inflatable balloon 120 can vary depending on the embodiment. For example, in one embodiment, the expandable basket 130 and the inflatable balloon 120 can move together, while in another embodiment, the basket 130 can move independently from the balloon 120. For example, the basket 130 can be fixed in the rotational direction but can move in the axial (longitudinal direction) or in another embodiment it can be fixed.

The movement of the expandable basket 130 relative to the catheter body and the inflatable balloon 120 can be either an automated process as by using an electronic controller or it can be a manual process that occurs under action of the user. The controls permit the desired movements in rotational and/or longitudinal directions.

Delivery of energy and electrode selection: In one embodiment, energy is delivered to two or more electrodes 150 that are located along the same spline 140. In this embodiment, since the distance between the electrodes 150 on one spline 140 is fixed and does not change based on the expansion of the basket. This allows the PFA dosing to be selected since the distance between the electrodes 150 to be activated is known. In another embodiment, energy is delivered between two electrodes 150 that are not located along the same spline 140 but rather are located along adjacent splines 140. In this case, the distance between the splines 140 does change depending on the degree of basket expansion. For example, the greater the degree of basket expansion, the greater the distance between the splines 140 and thus, the greater the distance between the electrodes 150. When the electrode spacing remains fixed, there is a greater degree of dosing predictability.

In view of the visualization information and the location of and spacing between the electrodes that are to be actuated to cause lesion formation, the (PFA) dosing is selected. The correct (optimal) dose is one which provides good tissue isolation but does not adversely affect tissue quality.

In ablating tissue, certain select electrodes 150 are activated as opposed to activating all of the electrodes 150. Only those electrodes 150 that are in direct contact with tissue are activated to deliver energy and form the tissue lesion.

Depending upon the visualization information, the basket 130 may need to be moved axially and/or rotationally in order to perform the ablation. For example, if the electrode spacing is too great, energy is delivered to form a first lesion segment and then the basket may need to be moved relative to the balloon (axially and/or rotationally) to reposition the electrodes and deliver energy to form a second lesion segment that is combined with the first lesion segment to form a more complete lesion segment. Alternatively, circumferential electrode spacing can be inferred from the endoscopic view and the PFA dosing adjusted to compensate for differing electrode spacing.

The shape and size of the formed lesion segment will depend on which electrodes were actuated and their locations. For example, activation of two electrodes 150 located along the same spline 140 will result in a formed lesion that extends more longitudinally, while activation of two electrodes 150 located along adjacent splines results in a formed lesion that extends more in a circumferential direction.

### Dual Transeptal/Secondary Catheter

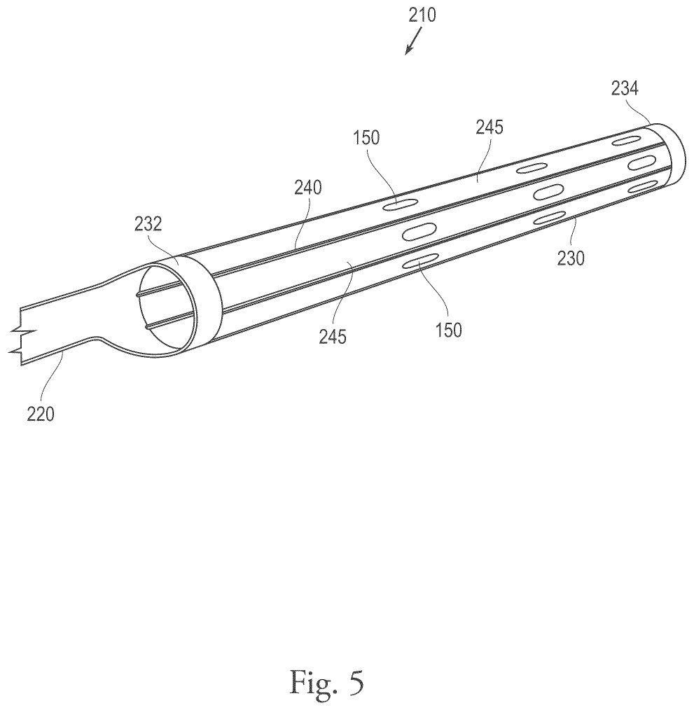

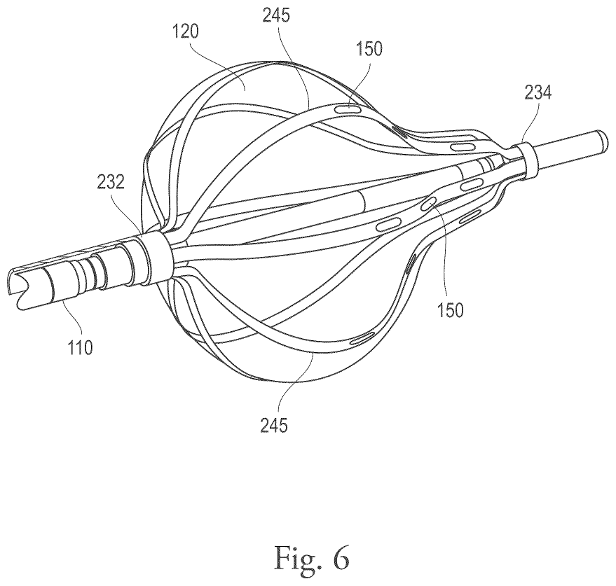

FIGS. 5 and 6 illustrate a balloon catheter 200 that is similar to the balloon catheter 100 with the exception that the balloon catheter 200 does not include the expandable basket 130. As a result, the reference numbers used in FIG. 4 are also used in FIGS. 5 and 6 for the parts that are in common to the two embodiments. The inflatable balloon is typically transparent and thus FIG. 6 shows the transparent nature of the balloon.

The balloon catheter 200 includes the main catheter shaft 110 that typically includes more than one shaft and often includes an inner catheter shaft and an outer catheter shaft or can otherwise include multiple concentric tubular structures as shown. The inflatable compliant balloon 120 is included and is coupled to the main catheter shaft 110 with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

In this embodiment, there is a second catheter, namely, an electrode catheter 210 that is for use with the balloon catheter 200. The electrode catheter 210 comprises an elongated structure that has an open distal end and has a proximal region 220 and a distal electrode region 230. The proximal region 220 can comprise an elongated arcuate shaped body that is not completely circumferential in shape. Conversely, the distal electrode region 230 can be a completely circumferential structure. The distal electrode region 230 includes a proximal collar 232 at a proximal end of the distal electrode region 230 and a distal collar 234 at a distal end of the distal electrode region 230. Between the two collars 232, 234, the body of the distal electrode region 230 includes a plurality of longitudinal slits 240 that are spaced circumferentially about the body. These slits 240 define a plurality of longitudinal splines 245. The slits 240 do not extend into the areas of the two collars 232, 234. Much like the splines 140, the splines 245 carry one or more and preferably a plurality of the electrodes (e.g., electrodes 150) that are located along the outer surface (outer face) of the splines 245. Much like the previous embodiment, each spline 245 can carry a plurality of electrodes, such as three of more electrodes that are disposed in series and spaced apart from one another in the longitudinal direction of the spline 245.

Both ends of the distal electrode region 230 are open and thus, it represents an open-ended tubular structure that, as described herein, is configured to receive the balloon catheter in its contracted (deflated) at rest state.

As in the previous embodiment, the splines 245 are not elastic and thus do not stretch but can expand in response to the expansion of the inflatable balloon 120. Therefore, the distance between the electrodes along the same spline 245 do not change based on whether the spline 245 is expanded or retracted. However, as in the previous embodiment, the distance between two electrodes on two different splines 245 does change based on the degree of expansion.

The balloon catheter is inserted into and through the hollow interior (inner lumen) of the electrode catheter 210 such that the splines 245 surround the inflatable balloon 130. As the balloon inflates, the splines 245 expand radially outward and separate from one another.

As in the other embodiment, the splines 245 can collapse as by retracting the splines 245 inside a main (outer) catheter shaft.

Visualization is used in this embodiment also to determine which electrodes are in contact with the tissue and also the visualization can guide the user in terms of making any adjustments with the balloon catheter and/or the electrode catheter in order to form a complete continuous lesion.

### Retractable Tines Electrode Array

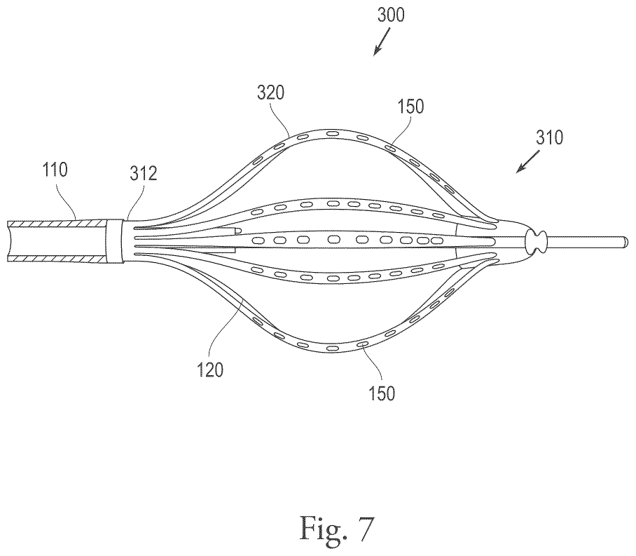

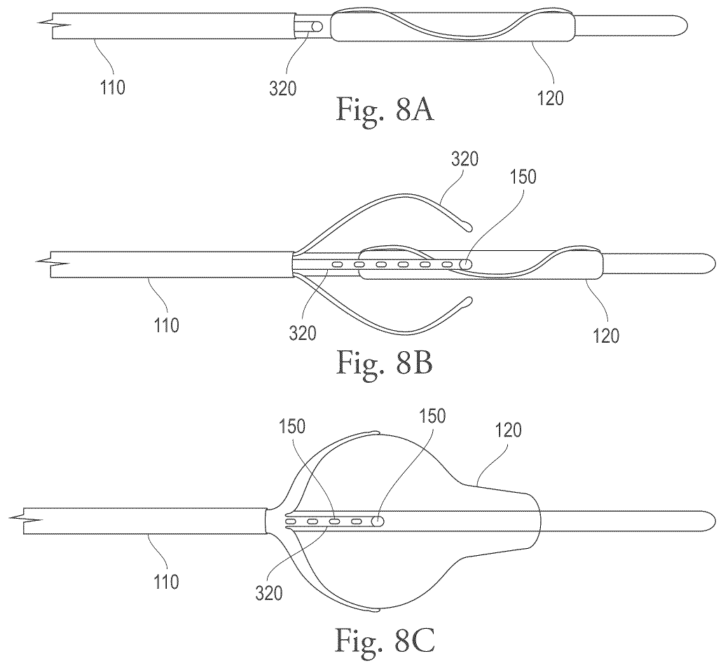

FIGS. 7 and 8A-C illustrate a balloon catheter 300 that is similar to the balloon catheter 100 with the exception that the balloon catheter 300 does not include the expandable basket 130. As a result, the reference numbers used in FIG. 4 are also used in FIGS. 7 and 8A-C for the parts that are in common to the two embodiments.

The balloon catheter 300 includes the main catheter shaft 110 that typically includes more than one shaft and often includes an inner catheter shaft and an outer catheter shaft or can otherwise include multiple concentric tubular structures as shown. The inflatable balloon 120 is included and is coupled to the main catheter shaft 110 and/or an additional shaft with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

The balloon catheter 300 further includes a retractable electrode sheath 310 that is configured to retract within the main catheter shaft 110 or another shaft of the catheter. Thus, as described herein, the retractable electrode sheath 310 is designed to move longitudinally along the main catheter shaft 110 and more particularly, the retractable electrode sheath 310 can travel within the main catheter shaft 110 to allow the retractable electrode sheath 310 to move between a fully retracted position and a fully extended position. In the fully retracted position, at least a substantial length of the retractable electrode sheath 310 is contained within the main catheter shaft 110 and in the fully extended position, a substantial length of the retractable electrode sheath 310 is disposed outside of the main catheter shaft 110 and surrounds the inflatable balloon 120 as described herein. As shown, in the fully extended position, the tines 320 can extend at least 75% of the length of the balloon 130 and can extend over 90% of the length of the balloon. In another embodiment, the tines 320 extend at least 50% of the length of the balloon 130 (e.g., they extend at least to the widest part of the inflated balloon 130).

The retractable electrode sheath 310 includes a proximal collar 312 that can be a continuous cylindrical structure and a plurality of expandable tines 320 that are integral at their proximal ends to the proximal collar 312. The tines 320 are cantilevered structures in that a distal end of each tine 320 is a free end and is not attached to another structure. The tines 320 are spaced apart and extend circumferentially around the balloon 130 when in the fully extended positions.

As in the other embodiments, the tines 320 are not elastic and do not stretch in any way; however, the tines 320 are able to expand (radially) outward as the inflatable balloon 130 inflates and similarly, when the inflatable balloon 130 deflates, the tines 320 can contract. The tines 320 thus can conform to the compliant balloon 130.

To cause retraction and full collapse of the tines 320, the retractable electrode sheath 310 is pulled back in the proximal direction and as the retractable electrode sheath 310 enters into the main catheter shaft 110, the presence of the main catheter shaft 110 in surrounding manner, applies an inward force to the tines 320 that collapses them and allows them to travel within the main catheter shaft 110 and retract away from the balloon 130.

As shown in the figures, each tine 320 includes one or more electrodes 150 and preferably a plurality of electrodes 150 that are spaced along the tine 320. The electrodes 150 are disposed in series along the length of the tine 320. The electrodes 150 along the tine 320 can be of the same type (e.g., same shape and size, etc.) or different type electrodes can be used in another embodiment.

As in the other embodiments, visualization (e.g., the endoscope) is used to determine which electrodes 150 are in contact with the tissue and those select electrodes can then be activated (actuated) to form the lesion. The user interface allows for the identification and powering of those electrodes 150 that are in contact with the tissue. As mentioned previously, the operating software can be programmed so that based on the distance between the activated electrodes 150, the proper dosing amount can be calculated and the requisite energy can be delivered to the electrodes 150.

As in all embodiments, it is desirable to limit the activation of electrodes to only those that are required to form the lesion (segment).

FIG. 8A shows the inflatable balloon 130 in a deflated state and the tines 320 are fully retracted and are located substantially within the main catheter shaft 110 (e.g., only the tips of the tines 320 protrude outside of the main catheter shaft 110).

FIG. 8B shows the inflatable balloon 130 still in its deflated state but the tines 320 have been deployed. As mentioned, the degree of coverage of the tines 320 relative to the balloon 130 can vary.

FIG. 8C shows the balloon 130 inflated and this results in the expansion of the deployed tines 320. In these figures, the tines 320 are shown extended about 50% the length of the balloon 130; however, this is merely exemplary in nature and it will be understood that it can extend along more or less of the balloon length.

The embodiment of FIGS. 7 and 8A-C thus consists of semi-rigid retractable tines 320 with one or more electrodes 150 along the outer surface of each tine 320, that are housed within the catheter (main catheter shaft 110) and deployed before inflating the balloon 130 (by sliding the retractable electrode sheath 310 distally using a controller or the like (manual or motorized). When the balloon 130 is inflated, the electrodes 150 are pressed against the inner surface of the vessel to achieve tissue contact. As with the other embodiments, the endoscope in this embodiment within the balloon 130 to allow for confirmation of tissue contact and electrode spacing under direct visualization, once tissue contact and desired electrode spacing is confirmed, energy is applied to the desired (selected) electrodes 150 to create the lesion. This embodiment can incorporate as few as four deployable tines 320, but a larger number of tines 320 is likely to provide the user with the ideal number of electrodes 150 and electrode spacing for effective treatment.

In this embodiment, as in the other embodiments, markers can be provided along the inner surface of the tine 320 to identify the location of the electrodes 150 along the tine 320 under visualization. This allows the user to determine which electrodes 150 are in contact with the tissue and then instruct the energy delivery module to deliver energy to those selected electrodes 150. In addition, in one embodiment, the system can include image recognition software that analyses the live image feed from the endoscope and identifies the electrode markers that are present. For example, if the markers, such as A1 and A2, are present, then the image recognition module will identify these electrodes and provide the user with the option to confirm that the electrodes that correspond to markers A1 and A2 should be activated and energy delivered to the user.

This image recognition functionality can be implemented in any of the other embodiments described herein in which the electrode markers are present to provide the user with a suggested electrode activation plan.

Balloon with PFA Braided Wire Mesh Electrode Array

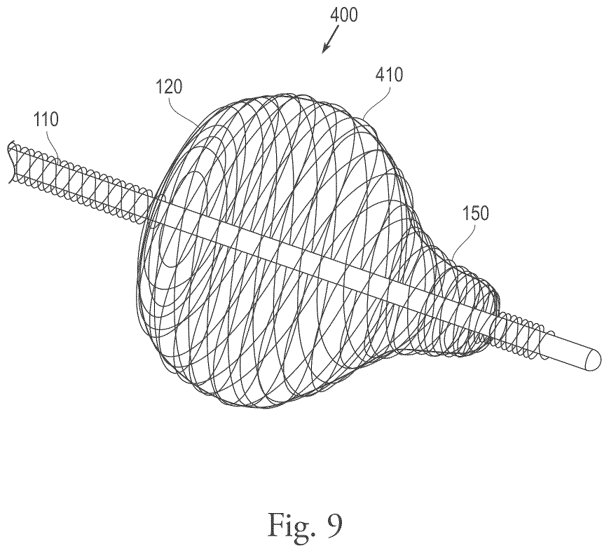

FIG. 9 illustrates a balloon catheter 400 that is similar to the other balloon catheters described herein. As a result, the reference numbers used in FIG. 4 are also used in FIG. 9 for the parts that are in common to the two embodiments.

The balloon catheter 400 includes the main catheter shaft 110 that typically includes more than one shaft and often includes an inner catheter shaft and an outer catheter shaft or can otherwise include multiple concentric tubular structures. The inflatable balloon 120 is included and is coupled to the main catheter shaft 110 with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

The balloon catheter 400 includes a wire braid 410 that is disposed over the inflatable balloon 120 and is configured to expand radially as the inflatable balloon 120 is inflated. The wire braid 410 can comprise a mesh wire braid as shown. This wire mesh can be used as a support structure for an electrode array formed of electrodes 150 and can be formed of an insulating material. The electrodes 150 are disposed along the outer surface of the wire braid 410 and the coverage of the electrodes 150 can be uniform of non-uniform. In the non-uniform embodiment, the electrodes 150 can be more concentrated in one or more regions of the wire braid 410. For example, the electrodes 150 can be primarily located at the center region of the wire braid 410 where tissue contact is more likely.

In addition, the spacing between electrodes can be the same along the entire electrode array or the spacing can be different in one or more regions of the wire braid 410. For example, the spacing can be closer together in a central region of the wire braid 410.

As in the other embodiments, the electrodes 150 are connected to the energy source using conventional electric traces or wires (conductive paths) that are associated with and/or incorporated into the wire braid.

Alternatively, the wire braid 410 (support structure) itself can serve as and define the electrode array by incorporating an insulating coating on a conductive (metal) braid wires that is stripped off at desired locations for energy delivery by defining discrete electrodes in those area where the coating is removed. The wire braid 410 would be operatively connected to the energy source and electric current (energy) is delivered across the wire braid 410 with the areas in which the insulating coating is removed defining the electrodes that define the electrode array.

The wire mesh braid can be formed of separate discrete insulated wires to define discrete pathways along which the electrodes are present. By defining discrete electrode pathways, discrete regions of the wire mesh braid can be activated without activation of the other regions to allow for activation of those electrodes or that electrode region that is in contact with the tissue.

As shown, the wire braid 410 can extend beyond the inflatable balloon 130 in that one end of the wire braid 410 extends proximal to the inflatable balloon 130 and the other end of the wire braid 410 extends distal to the inflatable balloon 130.

As with the other embodiments, this embodiment once again uses an endoscope inside the balloon 130 in order to confirm electrode placement and tissue contact. The number of electrodes 150 in the array may vary along with the number of braid wires in order to achieve the most clinically effective energy delivery, and the user may be able to select or deselect a number of electrodes in order to customize the treatment zone.

Balloon with Embedded Electrode Array

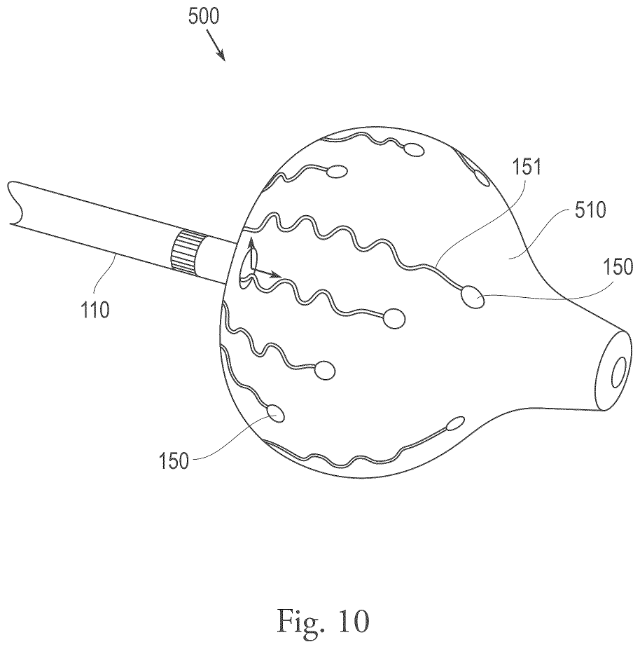

FIG. 10 illustrates a balloon catheter 500 that includes the main catheter shaft 110 along with an inflatable balloon 510 that is coupled to and extends along the main catheter shaft 110 as in the other embodiments. The distal end of the inflatable balloon 510 is coupled to a distal end of the main catheter shaft 110 and a proximal end of the inflatable balloon 510 is coupled to the main catheter shaft 110 at a location spaced from the distal end of the main catheter shaft 110.

The inflatable balloon 510 is a compliant balloon in which the electrodes 150 are integral. The balloon 510 itself includes electrodes 150 and flexible wire traces 151 embedded in the balloon material.

In this embodiment, the electrodes 150 can be disposed in and made integral to the balloon 510 as part of the molding process of the balloon 510. The electrodes 150 are spaced across the balloon 510 in a desired pattern. For example, the electrodes 150 are located circumferentially around the balloon 510. Alternatively, instead of being positioned and attached to the balloon material during the manufacture process, the electrodes 150 can be attached to the balloon 510 after the manufacture process. In particular, the electrodes 150 can be attached to the outer surface of the balloon 510 with the traces 151 also being attached to the outer surface of the balloon 510. Any number of conventional techniques can be used to attach these elements to the outside of the balloon 510 such as use of adhesive, bonding agents, etc.

The electrodes 150 are formed so that an outer surface of each electrode 150 is exposed along the surface of the balloon 150 for placement in contact with the tissue. Each flexible trace 151 is formed in a zig-zag pattern which is purposeful in order to permit the flexible traces 151 to move with the compliant balloon during inflation/deflation and during placement against the tissue. In other words, this zig-zag pattern accommodates the flexible traces 151 during the expansion and contraction of the balloon and prevents damage to the trace(s). Each flexible trace 151 is operatively coupled to the energy source so that energy can be delivered to select ones of the electrodes 150.

As with the other embodiments, this embodiment once again uses an endoscope inside the balloon 510 in order to confirm electrode placement and tissue contact. Once the user determines which electrodes 150 are in contact with the tissue, the user can then select these electrodes for activation.

In addition, electrode markers can be provided as in the other embodiments that are visible on the inside of the balloon 510 to the endoscope to allow the user or to allow image recognition software to determine which electrodes are clearly visible in the field of view of the endoscope. Based on this information, energy is delivered to those select electrodes 150 for forming the lesion. The user interface can be configured to easily allow the user the ability to select which electrodes to deliver energy to as by presenting the user with a touch screen with an electrode map and/or having the image recognition software prepopulate the screen with a proposed electrode activation map indicating which electrodes are visible in the endoscope and in contact with tissue.

Balloon with Micropores and Inner Electrode Array

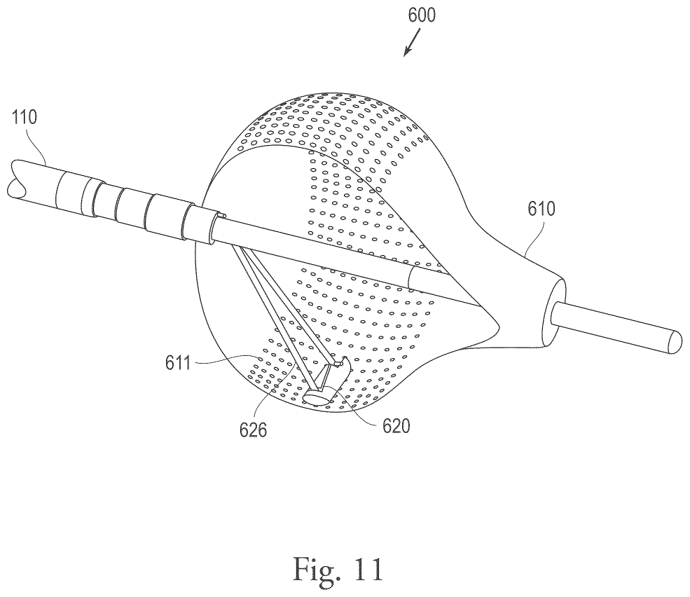

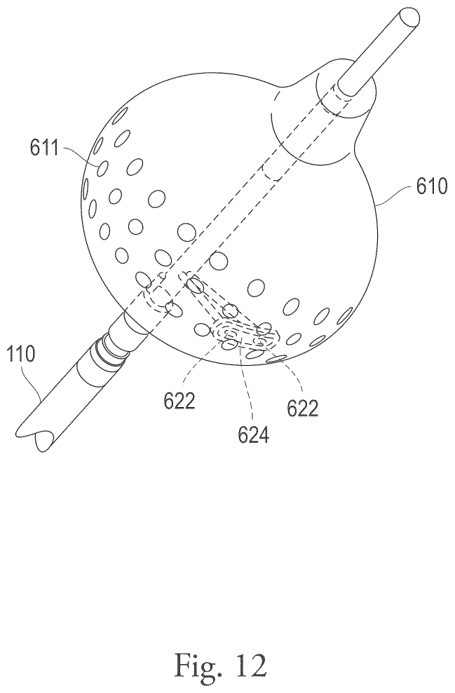

FIGS. 11 and 12 illustrate a balloon catheter 600 that includes the main catheter shaft 110 along with an inflatable compliant balloon 610 that is coupled to and extends along the main catheter shaft 110. An outer catheter body or sleeve 115 is also present and as mentioned, the catheter 600 can include other shafts, such as outer and inner catheter shafts, etc. The distal end of the inflatable balloon 610 is coupled to a distal end of the main catheter shaft 110 and a proximal end of the balloon 610 is coupled to the main catheter shaft 110 at a location that is spaced from the distal end.

As with the other embodiment, an endoscope is provided inside of the balloon 610 and can be coupled to the main catheter shaft 110. The endoscope is forward looking and allows view of the transparent balloon 610 and its contact with surrounding tissue.

In accordance with this embodiment, at least a portion of the balloon 610 has micropores 611 formed therein. The micropores 611 are preferably formed in one or more regions of the balloon 610 in which energy is to be delivered to the tissue. In the illustrated embodiment, the proximal and distal ends of the balloon 610 are devoid of micropores 611, while the center region includes the micropores 611 since it is this center region that contacts the tissue during use.

For ease of simplicity, the micropores 611 in FIG. 12 are shown as having greater dimensions than the micropores in FIG. 11; however, it will be understood that the micropores in FIGS. 11 and 12 can be the same size and the same number. However, FIG. 12 does convey that the micropores 611 can be formed to have different sizes and even different shapes.

The micropores 611 can have uniform constructions (i.e., same size and shape) or there can be two or more types of micropores 611. The micropores 611 can be formed in a uniform pattern as shown or can be formed in a non-uniform pattern. For example, as illustrated, the micropores 611 can be formed in a grid that extends circumferentially around the entire balloon 610.

The balloon catheter 600 also includes an electrode carrier 620 that is disposed within the balloon and can, in at least one embodiment, move within the balloon 610 (i.e., move rotationally within the balloon 610 and/or move longitudinally within the balloon 610). The electrode carrier 620 includes one or more electrodes 622 that are contained in a housing (hood) 624. In the illustrated embodiment, there is a pair of electrodes 622 in the housing 624 (however, it is possible to use a single electrode in the hood, with the hood rotating within the inside of the porous balloon). The housing 624 serves to contain and direct the energy of the electrodes 622. The electrodes 622 are placed in close proximity to the balloon itself, and the housing itself is placed in direct contact with the balloon's inner surface. The hood 624 can optimize the fraction of ablative energy delivered to tissue; however, the hood 624 can be eliminated and is not necessary.

The electrode array 622 is thus contained in the housing 624 which also serves to encapsulate a conductive liquid media, such as saline (e.g., normal saline or hypertonic saline) that allows for energy flow directly into the tissue via the micropores 611. In other words, the conductive liquid media can be delivered to the housing 624 as by use of one or more conduits 626 that open up into the inside of the housing 624. When the electrodes 622 are activated, energy is produced by the electrodes (e.g., between the electrodes) and since the electrodes 622 are bathed in the conductive liquid media, the energy serves to heat the conductive liquid media. The presence of the micropores 611 allows the heated conductive liquid media to weep through the micropores 611 to the tissue, which in combination with the energy from the electrodes 622 being conducted across the balloon material results in a target lesion being formed. In particular, a lesion segment is formed. To form a complete lesion, the electrode carrier 620 can be rotated and/or moved along the inner surface of the balloon. The electrode carrier 620 is held in contact with the inner surface of the balloon 610 by means of a mechanical adjustment controlled by the user, or a secondary balloon that can be inflated or deflated by the user to adjust the electrode contact pressure.

The combination of the electrode array and the conductive liquid media defines an electroconductive pathway used to form the lesion segment. It will be appreciated that the inflation media to control inflation or deflation of the balloon 610 can be the same or different than the conductive liquid media delivered to the inside of the housing 624.

In yet another embodiment, the balloon 610 does not include micropores 611 but instead is formed of a conductive balloon material (e.g., balloon material doped with carbon nanotubes). In this alternative embodiment, the housing (hood) can also be eliminated or it can be maintained. A non-conductive fluid can thus be used inside the balloon. The electrode array (or single electrode) is still disposed inside of the balloon 610 and is movable therein as by being able to freely rotate within the balloon and/or move longitudinally. Energy delivered to the electrode array is thus transferred to a local region of the conductive balloon that is in close proximity to the electrode array to form the lesion. In other words, the electrode array faces a localized area of the balloon and energy that is delivered to the electrode array is conducted to this localized area of the balloon to form the lesion.

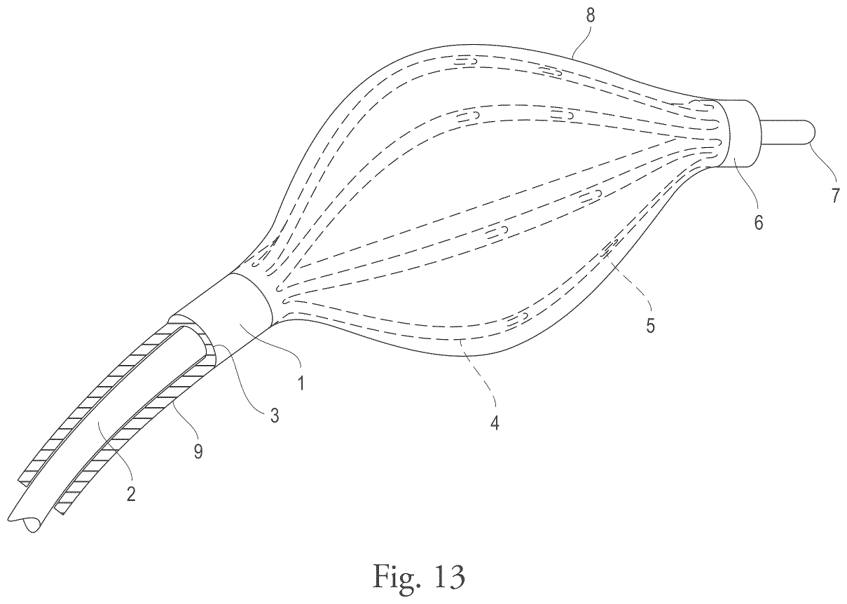

Now referring to FIG. 13, in yet another embodiment, a porous balloon catheter 700 is shown. The porous balloon catheter 700 is similar the balloon catheter 600 and therefore, like elements are numbered alike. The balloon thus includes micropores 611. Instead of the electrode carrier 620, the balloon catheter 700 includes an elongate structure 710 that can be similar to the elongate structure 1 of FIG. 1 with several notable differences being that the elongate structure 710 is located inside the balloon as opposed to being located outside the balloon as in FIG. 1. The elongate structure 710 comprises a first tubular shaped portion 712 and a second tubular shaped portion 714 that surround the catheter shaft. The elongate structure 710 multifurcates into two or more but preferably six or more branches 720, with each branch 720 containing one or more electrodes 715 on their outward facing surface. The elongate structure 710 can be made of an elastic material pre-shaped into a geometry that allows it to expand and remain in contact with the balloon inner surface as the balloon is inflated. The elongate structure 710 would be collapsed by the balloon when the balloon is deflated by removing liquid from the balloon under vacuum. In other words, as the balloon is inflated, the elongate structure is constructed to automatically and naturally expand and similarly, it contracts due to the contraction of the balloon. This can naturally occur due to the memory characteristics of the elongated structure 710. The electrodes 715 that are on the outer surface of the elongate structure 710 are thus in contact with the inner surface of the porous balloon. As in the other embodiment, the balloon contains conductive fluid that passes through the micropores. Thus, energy from the electrodes 715 is conducted across the balloon itself and/or the conductive fluid within the balloon passes through the micropores to the target tissue.

It will be appreciated that in all embodiments, the electrodes are connected to a controllable energy source using conventional techniques, including electric leads, wires, conductive pathways, etc. The energy source can be controlled using traditional controls such as a master controller that can be a part of a console at which the user enters input and can control and select different operating parameters such as dosing information (dose power (wattage), etc.

Those embodiments that incorporate an electrode array are particularly suited for delivery of electroporative ablation energy (PFA).

Additional details concerning certain embodiments of the present disclosure are as follows.

A device for the alteration of tissue for the purpose of changing, amongst other things, the conducting properties of the tissue to achieve a desired result.

An external sheath that is positioned over the existing catheter system.

Consisting of three distinct portions, a location collar of a hard material at the most distal end, a balloon expandible section of softer, more pliable material (or alternative arrangement) located in the vicinity of the primary balloon and an overcoat on the body of the catheter continuing until near the proximal end.

Electrodes may be placed on the hard collar section for measurement of distal electrical activity or may be employed in the delivery of energy.

Electrodes are primarily placed on the balloon expandible section, for the delivery of energy to achieve the alteration of the properties of the target tissue, in a variety of configurations (another section)

The overcoat of the body incorporates the conductors for the distal measurement and energy delivery and are terminated in the vicinity of the control for the rotation of the other energy delivery source.

The electrodes on the collar may be in a variety of configurations, including square electrodes in a 2,4, or 6 style equally spaced around the measuring area on the collar.

The balloon expandible area electrodes are intended to be the primary energy delivery (therapeutic) of the device. The most likely embodiment would be 16 electrode arrangement, equally spaced, positioned proximally to the primary treatment area, allowing the balloon to be deflated slightly to allow the electrode array to be extended distally into the area to be treated, perhaps, but not necessarily in an arc similar in location to where the primary energy was or will be delivered. The area will be aligned so that at the inflation pressure designated for “PFA” therapy, the electrodes will be equally spaced and are separate, so that they may be accessed individually or in a variety of groupings.

The overcoat of the catheter will have the conducting means for all of the sensing and energy delivery electrodes (some or all of them serve dual purposes) so as not to provide any, or at least a minimal amount, of impingement on the flexure or rotation of the primary catheter. This may be a spiral routing with the ability to use a variety of spiral pitches.

The balloon expandible section of the device may be a complete sheath consisting of a very elastic material with the electrodes on the surface or may be more rigid with sections of the device removed so that the electrodes are placed into the desired area by displacement of the structure.

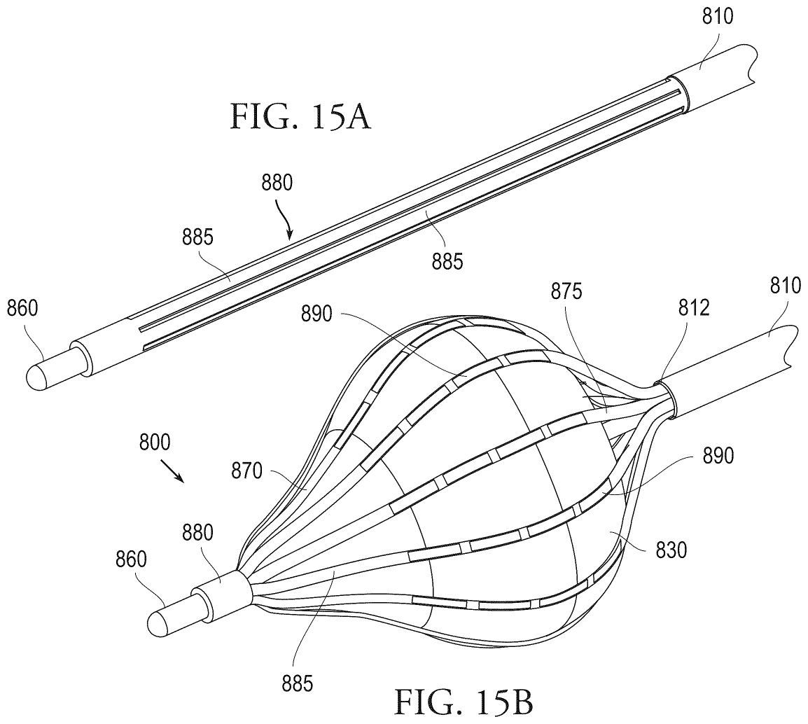

### Translating Tip Balloon Catheter (FIGS. 15A and 15B)

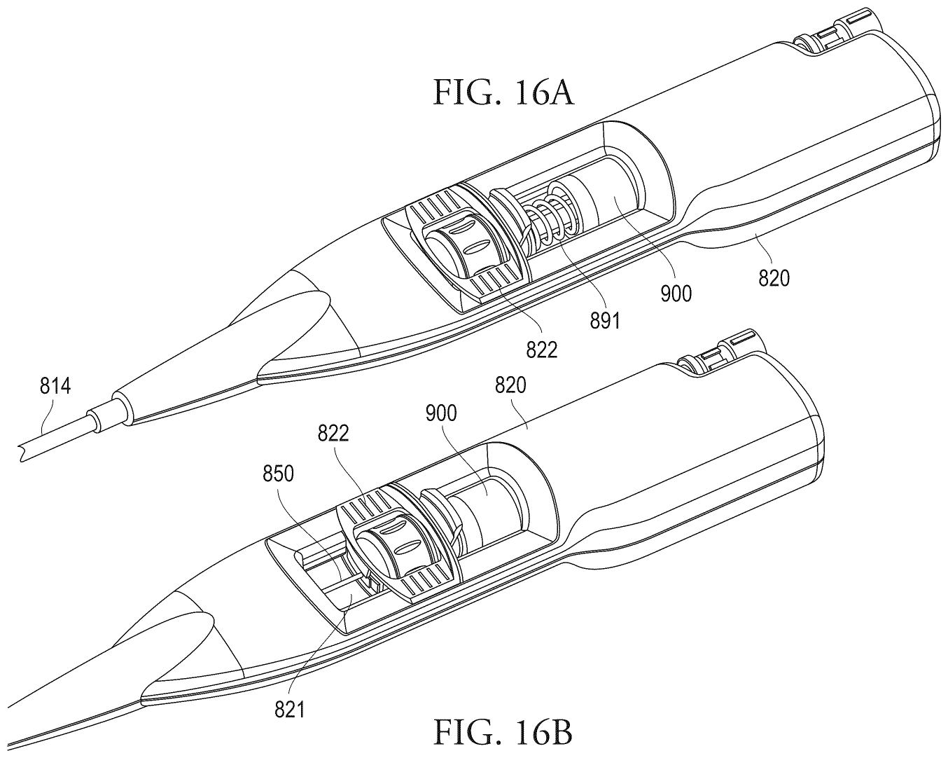

Now referring to FIGS. 15A and 15B in which a translating tip balloon catheter 800 is illustrated. FIG. 15A shows the catheter 800 in a collapsed state for delivery to the target site, while FIG. 15B shows the catheter 800 in an in use, expanded state. The catheter 800 includes an elongated outer catheter (shaft) 810 that has a distal end 812 and an opposite proximal end 814 (FIG. 16A). The outer catheter 810 is an elongated hollow structure. The catheter 800 also includes a handle 820 (FIG. 16A) for grasping by the user. The handle 820 is coupled to the proximal end 814 of the outer catheter 810. Additional details concerning the handle 820 are set forth below.

The handle 820 can take any number of different forms including being formed of two parts that are attached to one another to define the hollow interior that houses the working parts of the translating tip balloon catheter 800.

The catheter 800 also includes an inflatable balloon 830 that is coupled to the distal end 812 of the outer catheter 810. The outer catheter 810 can terminate proximal to the balloon 830 or it can extend partially into the balloon 830; however, the outer catheter 810 does not extend completely to the distal end of the catheter 800.

The balloon 830 comprises a compliant balloon. It will be appreciated that the catheter 800 and balloon 830 have traditional inflation and deflation architecture such as an inflation and/or deflation lumen through which inflation media flows into the balloon for inflation thereof. As is known, the inflation media can be circulated using a pump or the like.

The catheter 800 is configured to deliver PFA energy using an expandable electrode basket construction that surrounds the balloon 830 along with an actuator or translation mechanism that allows the electrode basket construction to both expand and collapse to a more flattened state. More specifically, the translation mechanism can include an elongated structure such as an actuator shaft 850 that can be a tube or solid rod 850 (FIG. 16B) that is coupled at a first (proximal) end to the handle and is coupled at an opposite second (distal) end to a flexible nose tip 860. Thus, while the element 850 is described as being a tube, it will be appreciated that it does not have to have a tubular structure but can be solid. In the present disclosure, the expressions “actuator shaft 850” and “tube 850” and “nitinol tube 850” are used interchangeably.

In one embodiment, the actuator shaft 850 comprises a nitinol tube.

The nose tip 860 defines the distal end of the catheter 800. The nose tip 860 is not directly attached to the outer catheter 810 but instead is capable of axial movement thereto and this provides the axial translation aspect of the catheter 800. The distal end of the tube 850 is thus fixedly attached to the nose tip 860 and passes through the inside of the balloon 830. As a result, when the tube 850 is driven in a forward direction, the nose tip 860 is driven forward and conversely, when the tube 850 is driven in a rearward direction, the nose tip 860 is driven rearward towards the handle.

The expandable electrode basket, according to one embodiment, is formed of a first electrode basket 870 and a second electrode basket 880 that are described in more detail herein. Each of the first electrode basket 870 and the second electrode basket 880 is coupled to both the distal end 812 of the outer catheter 810 and to the nose tip 860. As illustrated, the first and second electrode baskets 870, 880 are layered in that the first electrode basket 870 can be considered to be an inner basket and the second electrode basket 880 can be considered to be an outer basket.

The first electrode basket 870 includes a distal end portion that can be in the form of a solid cylindrical portion and a proximal end portion that can also be in the form of a solid cylindrical portion. Similarly, the second electrode basket 880 includes a distal end portion that can be in the form of a solid cylindrical portion and a proximal end portion that can also be in the form of a solid cylindrical portion. The distal end portion of the first electrode basket 870 is coupled to and can surround the nose tip 860, while the distal end portion of the second electrode basket 880 can be disposed over the distal end portion of the first electrode basket 870 and thus surrounds (superimposed over) the distal end portion of the first electrode basket 870. Similarly, the proximal end portion of the second electrode basket 880 can be disposed over the proximal end portion of the first electrode basket 870 and thus surrounds (superimposed over) the proximal end portion of the first electrode basket 870.

Each of the first electrode basket 870 and the second electrode basket 880 carries one or more electrodes. As illustrated, the first electrode basket 870 and the second electrode basket 880 are splined structures in that the first electrode basket 870 includes a plurality of longitudinal slits that create and define a plurality of first splines 875 that extend circumferentially around the first electrode basket 870. The second electrode basket 880 includes a plurality of longitudinal slits that create and define a plurality of second splines 885 that extend circumferentially around the second electrode basket 880. As described herein, the locations of the first and second splines 875, 885 are purposedly selected by orientation of the first and second electrode baskets 870, 800 such that when the balloon 830 expands and the first and second splines 875, 885 move to their expanded states (FIG. 15B), the first and second splines 875, 885 do not overlap but are interposed within one another. In other words, each second spline 885 is located between two adjacent first splines 885 and vice versa.

In one embodiment, each of the first and second electrode baskets 870, 880 are constructed such that they have six (6) splines and as a result, the superimposed and offset nature of the first and second electrode baskets 870, 880 defines twelve (12) splines. The first and second electrode baskets 870, 880 can thus have the same or very similar structure with one fitted over the other and rotated so as to rotationally offset the splines of each. Since each spline can carry one or more electrodes, when the balloon is inflated and the splines 875, 885 are in the expanded state, the twelve splines are circumferentially spaced along the outside of the balloon and provide increased electrode coverage for contact with tissue. If only one electrode basket (one sheath or collar) was used, the inclusion of enough longitudinal slits to form twelve splines would result in each spline having insufficient width to carry the desired ablation electrodes (e.g., for PFA). The present arrangement of two superimposed electrode baskets that are rotationally offset overcomes this deficiency and permits twelve splines each of sufficient width to carry one or more electrodes of desired size suitable for PFA.

In the initial, collapsed state, the first splines 875 are at least substantially covered by the second electrode basket 880 and thus, in this initial, collapsed state, only the second electrode basket 880 is substantially visible since the first electrode basket 880 is covered.

In one embodiment, each spline 875, 885 carries one or more electrodes 890 (FIG. 15A omits the electrodes 890 for ease of illustration). In the illustrated embodiment, each spline 875, 885 includes four electrodes 890 that are spaced apart and arranged in series (longitudinally) along the spline. In one embodiment, each electrode 890 can be controlled independently or in another embodiment, all of the electrodes 890 on one spline 875, 885 can be controlled together. As is known, a controller is used to control the supply of the ablation energy (e.g., PFA) to the electrodes 890 and in some embodiments, visualization can be used to detect which spline electrodes 890 are in contact with the target tissue. In one embodiment, instead of applying energy to all electrodes 890, energy can be supplied to select spline electrodes 890 (e.g., those electrodes 890 in contact with the target tissue).

The first and second electrode baskets 870, 880 are not compliant like the balloon 830. In one embodiment, the first and second electrode baskets 870, 880 are formed of polyimide.

The electrodes 890 are secured to the splines using conventional techniques, such as bonding, etc. Each electrode 890 can be connected to the energy source with an electrical trace as is known. For example, the electrical traces can be copper traces and the electrodes 890 can be gold plated electrodes.

Now referring to FIGS. 15A-16B, the handle 820 includes a through opening 821 in which an actuator 822 is movable disposed and contained. For example, the actuator 822 can be in the form of a slider that can be accessed and manipulated on opposite sides (faces) of the handle 820. A forward end of the actuator 822 is fixedly coupled to the tube 850 and therefore, when the actuator 822 is moved axially, the tube 850 moves axially since they are fixed to one another (no relative movement therebetween). Thus, when the actuator 822 is moved forward, the tube 850 moves forward and this is translated into the nose tip 860 moving forward. Since the balloon 830 and each of the first and second electrode baskets 870, 880 are fixedly attached to the nose tip 860, all of these structures move forward as well. The forward movement results in flattening (elongation) of the first and second electrode baskets 870, 880 toward and to the position shown in FIG. 15A as a result of the balloon 830 being deflated.

It will however be understood that the actual expansion and contraction of the spline baskets 870, 880 are due to the inflation and deflation of the balloon 830. When the balloon 830 inflates, it will begin to pull on the splines. If the nose tip 860 is in the forward position and is preferably locked therein, there will be a lot of stress on the splines by the balloon 830. The balloon 830 is trying to stretch to a size and the splines 875, 885 resist it. As described herein, the locking of the actuator shaft 850 and nose tip 860 prevents any rearward movement of either to permit the deflated balloon and the splines 875, 885 to be in and remain in the contracted state until the delivery to the target site. When the actuator shaft 850 is moved rearward, the actuator shaft 850 moves rearward and this is translated into the nose tip 860 moving rearward. When the actuator shaft 850 and nose tip 860 move rearward as by action of the knob/slider, the stress of the splines lessens. It will be understood that this rear movement in and of itself does not cause expansion of the splines 875 885 but reduces the stress on the splines 875, 885 and the splines 875, 885 will appear to be somewhat loose/floppy but in this state, upon inflation of the balloon 830, the splines 875, 885 will expand outward as the ballon expands, thereby allow the splines 875, 885 to assume the position shown in FIG. 15B.

Additional details concerning the actuator 822 are found in U.S. Pat. No. 11,389,236, which is hereby incorporated by reference in its entirety.

In yet another aspect, the handle 820 includes a biasing mechanism that operates on the tube 850. In particular, the biasing mechanism can be in the form of a spring 891 that is contained within the handle and applies a biasing force to the rear of the actuator 822. The spring 891 can be a coil spring. One end of the spring 891 seats against a fixed surface of the handle, while the opposite end seats against the rear of the actuator 822. The spring 891 shown is designed to assist in some of these axial movements. To deliver the catheter 800 to the target location, the catheter 800 can be delivered through a delivery sheath (not shown). Going through the sheath, the distal nose tip 860 will be pushed in a proximal direction by the frictional forces of going into the sheath. Some force will be required to resist this movement, and the spring 891 provides that force. Once inside the patient and in place, the doctor will inflate the balloon 830, thereby causing expansion of the splines and electrodes.

In one implementation of this design, the spring 891 is designed such that the inflation of the balloon 830 is enough to overcome the spring 891 and pull the distal nose tip 860 backwards, allowing the splines 875, 885 to expand to the shape of the balloon 830 as the balloon 830 inflates. In another implementation, there will be some sort of lock that can be removed so that the spring 891 is either not pushing on the nitinol tube 850 or is pushing on it less. This is shown simply for now by a removable block 900 proximal to the spring 891. However, specific exemplary lock mechanisms are described below.

FIG. 16A shows the handle and the actuator 822 in an extended state and FIG. 16B is a perspective view of the handle of FIG. 16A in a retracted state. When the balloon 830 is deflated and the first and second electrode baskets 870, 880 are in the flattened state of FIG. 15A, the actuator 822 is in the position shown in FIG. 16A and conversely, when the balloon 830 is the inflated state of FIG. 15B, the actuator 822 is in the position shown in FIG. 16B. Thus, as the balloon 830 is deflated, at some point, the spring force of the spring 891 overcomes the applied force of the balloon 830 and the actuator 822 is driven to the extended position shown in FIG. 16A which is the rest position of the catheter when the balloon 830 is deflated and the first and second electrode baskets 870, 880 are in the flattened state of FIG. 15A.

It will also be appreciated that in another embodiment, the catheter 800 only includes a single electrode basket, namely the first electrode basket 870 with a plurality of first splines 875 that form a complete circumference. The second electrode basket 870 is thus eliminated. In one embodiment, the plurality of first splines 875 comprises 6 or more splines that are circumferentially spaced apart. Thus, the single basket embodiment will appear as shown in FIG. 15A in that there is one electrode basket coupled to the outer catheter 810 and to the nose tip 860 as described herein. It will be understood that in FIG. 15A, the outermost second electrode basket 880 is shown; however, in a single basket design, the second electrode basket of FIG. 15A would represent the single (only) electrode basket that can be described as being the first electrode basket since there is only one.

The single catheter basket design works exactly like the two-basket design in that the user manipulates the actuator 822 to cause axial movement of the nitinol tube 850 which results in axial movement of the nose tip 860 resulting in the single electrode basket moving between the flattened state (FIG. 15A) and the expanded state, like FIG. 15B, when the balloon 830 inflates.

In the single catheter basket design, the handle can be spring loaded as well as described herein.

It will be appreciated that in one embodiment, the number of splines can be more than two and the widths of the splines are sufficient to support the one or more electrodes that are disposed on one or more electrodes. As with the other embodiment(s), each spline 875 can include one or more electrodes spaced longitudinally therealong for tissue ablation (e.g., PFA).

The nitinol tube 850 serves as an axial push/pull rod in each of these embodiments whether there is one or two electrode baskets 870, 880 surrounding the balloon 830. The movement of the nitinol tube 850 is preferably controlled within the handle.

### Inner Lumen Management

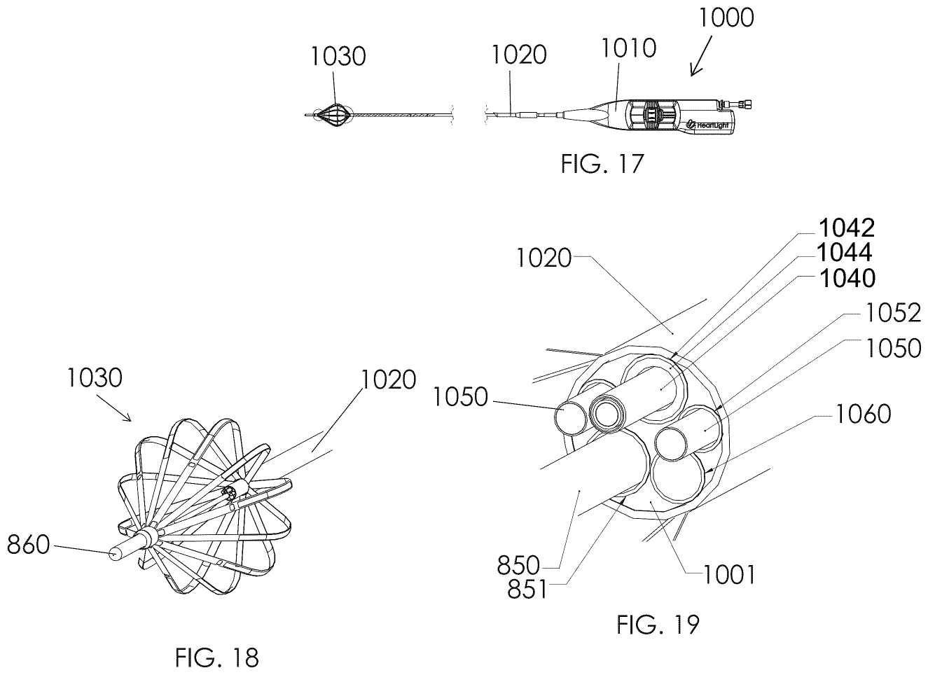

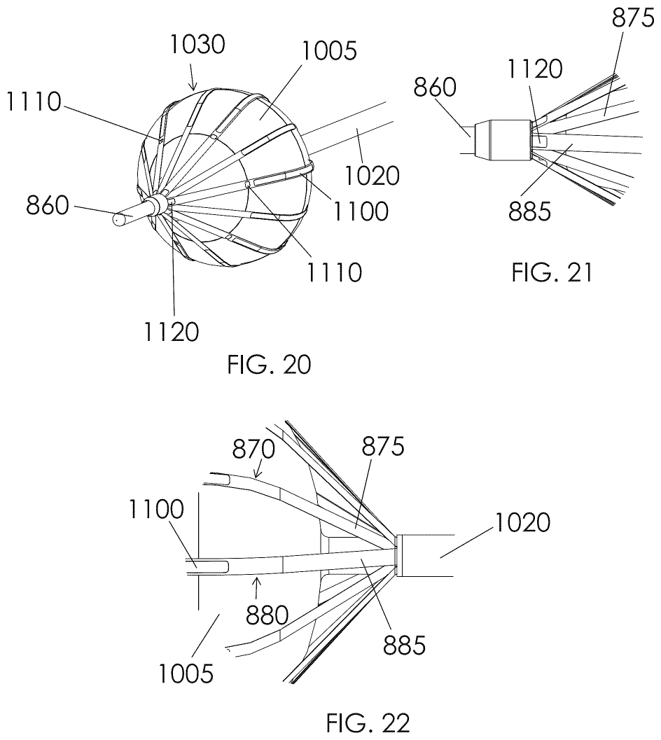

With reference to FIGS. 17-19, an inner lumen management construction is illustrated in relation to a catheter 1000. The catheter 1000 includes a handle 1010 along with an elongated shaft 1020, as well as an expandable basket 1030. It will be appreciated that the expandable basket 1030 can be used with an inflatable balloon 1005 when the catheter comprises a balloon catheter. Alternatively, the balloon can be absent in the case of a non-balloon catheter embodiment. It will be appreciated that in FIG. 18 the inflatable balloon is not shown only for ease of illustration but the balloon 1005 is shown in FIG. 20, as well as FIG. 17.

As will be understood, the catheter has a multi lumen construction to accommodate the working components and allow the different components to be routed along the length of the catheter and define fluid flow paths. As shown, the elongated shaft 1020 comprises and can be thought of as being a main lumen. The elongated shaft 1020 is a hollow tube and contains the other lumens and other working components of the catheter 1000 and extends from the handle 1010 to the expandable basket 1030.

The illustrated catheter 1000, like the other catheters (e.g., catheter 800) described herein, has visualization functionality in that it contains an endoscope 1040. The endoscope 1040 is contained within an endoscope lumen 1042 that is contained within the shaft 1020. The endoscope lumen 1042 can comprise a tubular structure that is separate from and is contained within the hollow interior of the tubular shaped shaft 1020. An endoscope stop 1044 is also provided and is configured to limit the movement of the endoscope 1040. The endoscope 1040 is a forward-looking device and is contained within the interior of the inflatable balloon at the proximal end thereof. The endoscope 1040 is thus fixed in its position.

The operation of the endoscope is described throughout the present disclosure and the drawings set forth representative figures showing endoscopic views generated by the endoscope. The use of the endoscope thus allows for live, real-time imaging of the surgical site to guide the surgeon and provide immediate feedback to the surgeon.

The catheter 1000 can also include one or more illumination devices 1050 and in the illustrated embodiment, there are a pair of illumination devices 1050 that are positioned within the main shaft 1020 opposite one another. In other words, the two illumination devices 1050 are generally positioned on opposite sides of the main shaft 1020. The illumination devices 1050 can comprise illumination fibers. Each of the illumination fibers 1050 is contained within an illumination lumen 1052.

FIG. 19 also illustrates a lumen 1060 that is used in the inflation and/or deflation of the inflatable balloon. For example, the lumen 1060 can be a deflation lumen. As described herein, a suitable fluid is delivered to the inside of the inflatable balloon for inflation thereof and conversely, fluid is removed from the inside of the inflatable balloon for deflation thereof.

The catheter 1000, like the other catheters, such as catheter 800, has an actuator or translation mechanism that allows the electrode basket 1030 construction to both expand and collapse to a more flattened state. More specifically, the translation mechanism can include an elongated structure such as the actuator shaft 850 (tube or solid rod 850), described hereinbefore, that is coupled at a first (proximal) end to the handle and is coupled at an opposite second (distal) end to the flexible nose tip 860 Thus, while the element 850 is described as being a tube, it will be appreciated that it does not have to have a tubular structure but can be solid. As mentioned, the tube 850 can comprise a nitinol tube. As shown, the tube 850 is contained within a tube lumen 851.

In the illustrated embodiment, the different lumens contained within the main shaft 1020 comprise different independent lumen structures (e.g., tubular structures routed within the main shaft 1020). It is possible in different embodiments that lumens could be formed as voids in the main shaft.

The balloon catheter 1000 thus has multiple lumens that help the catheter 1000 operate. The design uses fluid flow to inflate and deflate the balloon inside of the heart and this action allows a pressing of the flex strips (splines 875, 885/electrodes) up against the inside of the pulmonary veins. This fluid flow travels inside of the nitinol tube 850 and into the balloon, and travels out of the balloon through a braided polyimide tube (lumen 1060). The procedure is also endoscopically guided, and this is achieved by a fiber endoscope 1040 that is inserted into another braided polyimide tube (lumen 1042), and pokes into the balloon section for viewing. The endoscope 1040 requires light, and this is achieved through two light fibers 1050 that also poke into the balloon.

In accordance with the present disclosure, the catheter 1000 includes an inner lumen management feature and more specifically, the inner lumen management feature comprises a structure 1001 that keeps the various inner lumens in fixed positions within the main shaft 1020. For example, it is desired to separate the tube 850 from the endoscope 1040 and more particularly, it is desired to maximize separation of the tube 850 from the endoscope 1040. In order to maintain this separation, the inner lumen management feature acts to fixedly hold the lumens in their desired relative locations within the main shaft 1020 so as to maintain the desired separation at all times.

In one embodiment, the structure 1001 comprises a potting compound. The various lumens (tubes) are aligned at the end of the main shaft 1020 and then are potted in place. This ensures that the relative positions of the lumens (tubes) are fixed in place at the end of the main shaft 1020. In this way, the tube 850 and the endoscope 1040 are separated a maximum distance within the main shaft 1020. In other words, the tube 850 and endoscope 1040 are located directly opposite one another (180 degrees) within the main shaft 1020.

In yet another embodiment, a machined or molded cap (e.g., structure 1001) is used and the lumens (e.g., the braided polyimide tubes) are glued into the rear (back) end of the cap, and the face between the balloon and main shaft 1020 is now made up of the cap. The cap needs to include the endoscope stop 1044, which is how the endoscope extension into the balloon region is controlled. For the other lumens, the cap just provides a clean termination of the main shaft 1020 that the different components travel through. The light fibers 1050 travel inside of braided polyimide tubes 1052, and are glued in place extending out of the cap.

FIG. 19 illustrates the structure 1001 in the form of a potting compound that forms a hardened substance around all of the inner lumens within the main shaft 1020.

It will be appreciated that other techniques can be used to maintain the relative positions of the inner lumens, especially, the actuation tube 850 and the endoscope 1040.

### Electrode Design for Ablating and Mapping Functions

Now referring to FIGS. 17 and 20-22, in which an exemplary electrode design for the ablation/mapping catheter 1000 is shown. The catheter 1000 is similar to catheter 800 and thus, identical parts are numbered alike.

As previously mentioned, the ablation catheter 1000 includes the handle 1010 that is connected to the expandable basket 1030 by main shaft 1020. In these figures, the inflatable balloon is shown at 1005.

Electrode design (shape, size, material) is critical to the overall function of the catheter. In particular, the electrodes that are part of the catheter 1000 need to perform multiple functions. In one embodiment, the catheter 1000 is a single shot PFA (pulsed field ablation) catheter and the electrodes on such catheter need to serve multiple functions: 1) ablation; 2) visualization on mapping systems; and 3) measurement of electrograms in the heart to assess treatment.

As described previously, in one embodiment, the expandable basket 1030 comprises a plurality of splines that can expand and collapse. It will be appreciated that the expandable basket 1030 can comprise any of the expandable baskets described herein. For example, the expandable basket 1030 can comprise the superimposed first electrode basket 870 and the second electrode basket 880 that are described previously herein or it can be a single electrode basket defined by a plurality of splines.

As mentioned, each of the first electrode basket 870 and the second electrode basket 880 is coupled to both the distal end main shaft 10102 and to the nose tip 860 which is part of the axial translation mechanism to cause the expansion and collapse of the spines. As illustrated, the first and second electrode baskets 870, 880 are layered in that the first electrode basket 870 can be considered to be an inner basket and the second electrode basket 880 can be considered to be an outer basket. The various constructions and features of the baskets 870, 880 have previously been described herein and therefore, for sake of brevity will not be repeated again. Additional details concerning the two baskets 870, 880 will be appreciated by viewing the other figures. It will also be appreciated that at least a portion of each spline can be secured directly to the balloon's outer surface (using conventional techniques, like bonding agents).

Each of the first electrode basket 870 and the second electrode basket 880 carries one or more electrodes. As illustrated, the first electrode basket 870 and the second electrode basket 880 are splined structures. In one embodiment, each of the first and second electrode baskets 870, 880 are constructed such that they have six (6) splines and as a result, the superimposed and offset nature of the first and second electrode baskets 870, 880 defines twelve (12) splines. The first and second electrode baskets 870, 880 can thus have the same or very similar structure with one fitted over the other and rotated so as to rotationally offset the splines of each basket 870, 880. Since each spline can carry one or more electrodes, when the balloon 1005 is inflated and the splines 875, 885 are in the expanded state, the twelve splines are circumferentially spaced along the outside of the balloon 1005 and provide increased electrode coverage for contact with tissue. If only one electrode basket (one sheath or collar) was used, the inclusion of enough longitudinal slits to form twelve splines would result in each spline having insufficient width to carry the desired ablation electrodes (e.g., for PFA). The present arrangement of two superimposed electrode baskets 870, 880 that are rotationally offset overcomes this deficiency and permits twelve splines each of sufficient width to carry one or more electrodes of desired size suitable for PFA.

For the ablation function on a twelve-spline single shot ablation balloon catheter (e.g., catheter 1000), the need is to create very wide and very deep lesions such that the lesion under one electrode is able to connect to the lesion under the neighboring electrode and create a continuous zone of ablation to avoid the need to reposition the balloon 1005. With a point source electrode, the field decreases proportional to the square of the distance as you move away from the electrode (1/r²), while with a line source electrode, the field decreases proportional to the distance (1/r). So optimal depth and width of lesion for a twelve-spline single shot balloon catheter is achieved with electrodes 1100 that approximate parallel lines, i.e. long, evenly spaced electrodes 1100. It will thus be seen that each of the splines carries one long electrode 1100 that acts as the ablation electrode. The electrodes 1100 are located along the area of the balloon 1005 that is intended for tissue contact and thus, as shown, the electrodes 1100 do not extend to the distal end of the balloon 1005 and also do not extend to the proximal end region of the balloon 1005.

For both the mapping visualization function and the electrogram functions, the need is to create high spatial resolution signals with minimal noise. The larger an electrode is the more it acts as an antenna picking up noisy signals from far field tissue. Therefore, the optimal electrode for the mapping and electrogram functions is a point source electrode or one that's as small as possible. Further, for the electrogram function one wants to measure signals from tissue distal to the ablation in order to assess the ablation.

In view of the foregoing, the PFA balloon catheter 1000 optimizes all of these functions by providing long parallel electrodes 1100 for ablation with small electrodes 1110 just distal to the long parallel electrodes 1100 to mark the position where treatment ends with high resolution and to gather electrograms with high SNR and an additional electrode 1120 on the tip of the catheter to mark the catheter tip position with high resolution. As shown, the electrode 1110 is thus located between the electrodes 1100 and 1120 but is typically closer to the distal end of the electrode 1100 than to electrode 1120.

In one embodiment, the small electrode 1110, which can be referred to as a mapping electrode, can be a 1 mm×1 mm (length/width) sized electrode. The long (ablation) electrode 1100 can be a 20 mm×1 mm sized electrode. On other words, in one embodiment, the long ablation electrode 1100 can have a length that is at least 20 times the length of the mapping electrode 1110; in another the long ablation electrode 1100 can have a length that is at least 15 times the length of the mapping electrode 1110; in another embodiment, and in yet another embodiment, the long ablation electrode 1100 can have a length that is at least 10 times the length of the mapping electrode 1110.

The ablation electrodes 1100 are located on all twelve splines 875, 885, while the mapping electrodes do not have to be located on all twelve splines. For example, the mapping electrodes 1110 can be located on every other spline meaning that there can be six mapping electrodes 1110 which is sufficient to map.

It will also be appreciated that at least a portion of the ablation electrodes 1100 can be directly mounted (attached) to the balloon 1005 itself. This direct coupling avoids an issue in that when the balloon 1005 is inflated, the distal sections of the long ablation electrodes 1100 can potentially lift (the distal sections are those closest to the tip). Direct bonding of other electrode regions can prevent lifting of those regions. The direct bonding can be achieved by bonding the splines to the outer surface of the balloon.

It will also be appreciated that in one embodiment, the electrodes 1100 can comprise tapered electrodes in that a width of one end (e.g., distal end) of the electrode is less than the width at the other end (e.g., proximal end). The rationale is that that when expanded, the middle of the inflatable balloon represents the greatest diameter of the balloon, while the two ends are less. Thus, since it is desirable to keep the spacing between adjacent ablation electrodes 1100 the same along the length of the balloon, the tapered electrodes 1100 have their larger width at the wider center region of the inflatable balloon and have their smaller width at the end (e.g., distal end) of the inflatable balloon that has a small diameter. This tapered construction thus has an objective to maintain adjacent electrode spacing along the changing diameter of the balloon.

In one embodiment, the width of each end of the electrode 1100 can be at least ½ the width of the center section of the electrode.

### Handle Construction and Actuation Mechanism

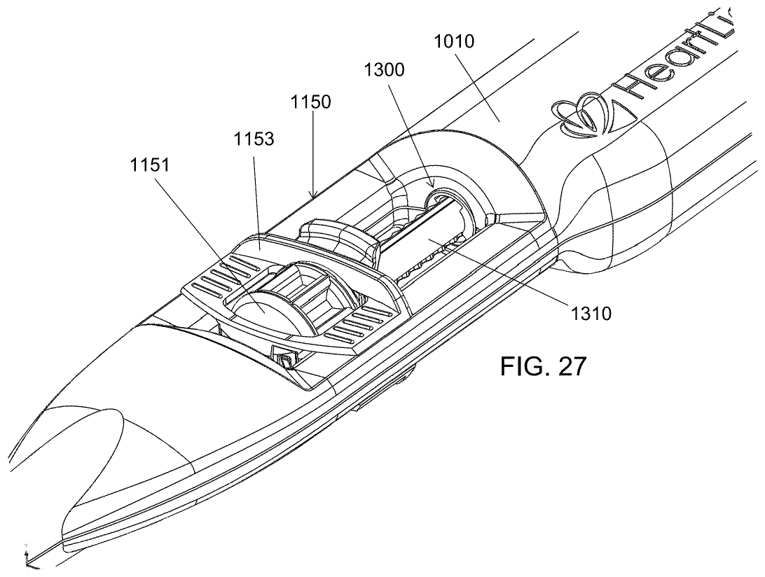

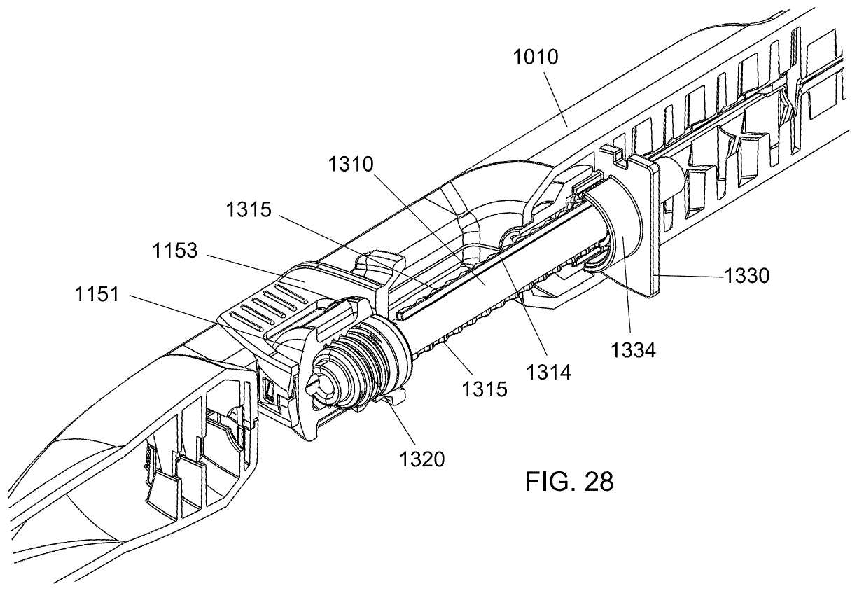

With reference FIGS. 27-28, the handle 1010 includes an actuator 1150 for controlling the deployment of the splines 875, 885 and more particularly, the expansion and contraction of the splines 875, 885. The catheter embodiment in FIG. 16A-B also has an actuator which is identified at 822 and can be the same or similar.

As previously mentioned, one of the major components of the actuation mechanism is the actuator shaft 850 that attaches to the nose tip 860 and since the nose tip 860 is coupled to the splines 875, 885, the axial movement of the actuator shaft 850 is translated into the movement of the splines 875, 885 between the expanded state (condition) and the collapsed state (condition).

The actuator 1150 is accessible to the user and is coupled to the actuator shaft 850, as described herein, and thus, manipulation of the actuator 1150 by the user results in axial movement and/or rotation of the actuator shaft 850. The handle 1110 includes a through hole or window that is defined as an open space between two opposing side walls or rails. In the illustrated embodiment, the actuator 1150 is defined by a knob 1151 and a slider 1153. Both the knob 1151 and the slider 1153 are disposed within the window. The knob 1151 is coupled to the actuator shaft 850 and therefore, rotation of the knob 1151 is directly translated into rotation of the actuator shaft 850 and axial movement of the knob 1151 within the window is translated into axial movement of the actuator shaft 850. The degree of travel of the knob 1151 is limited by the dimensions of the window in that the knob 1151 can only travel at most from one end of the window to the other end of the window. The slider 1153 comprises a sliding part that surrounds the rotating knob 1151. The rotating knob 1151 thus surrounds the actuator shaft 850 and can be a generally cylindrical shaped part that includes surface features, such as ribs or the like, that can be gripped by and manipulated by the user.

The slider 1153 is configured to slide along the two opposing rails that define the window. The slider 1153 is also configured such that the rotational movement (motion) of the rotating knob 1151 is not impeded by the surrounding slider 1153. The slider 1153 is constructed such that it can be easily contacted by the user to axially (longitudinally) advance the rotating knob 1151 and thus, axially move the actuator shaft 850.

As illustrated and as with the knob 1151, the slider 1153 is contained within the window of the catheter handle 1010 such that the two ends of the window define the ends of travel of the slider 1153.

Additional details concerning exemplary knob and slider constructions are set forth in US patent application publication No. 2024/0197395, which is hereby incorporated by reference in its entirety.

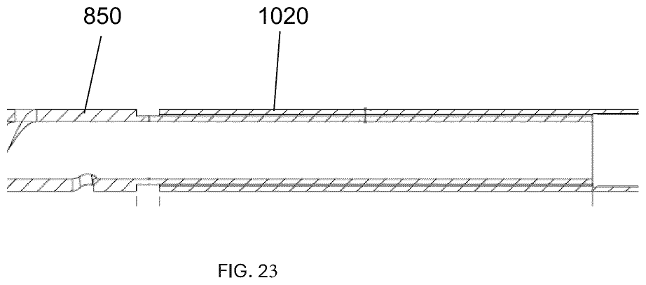

### Actuator Shaft (Nitinol Tube) Design (FIGS. 23-26)

As described herein, the catheters described herein, including catheters 800, 1000 utilize the actuator shaft 850 (e.g., a nitinol tube) that provides multiple functions to the overall catheter design. It will be understood that while the actuator shaft 850 is described herein as being a nitinol tube 850, other shaft materials and shaft constructions are possible and thus, referring to the actuator shaft 850 as being a nitinol tube is only exemplary and illustrative and not limiting of the scope of the present disclosure.

The actuator shaft 850 is utilized as a structural member in the spline/electrode basket end, providing enough rigidity and flexibility for the basket to be placed into veins. The distal tip of the catheter is bonded to the nitinol tube 850, connecting the tip (860) to the nitinol tube 850. The nitinol tube 850 rides inside of a braided polyimide tube (lumen 851) which is bonded to the main lumen catheter shaft 1020. The nitinol tube 850 only slides forward and back, so all other forces are translated between the nitinol tube 850 and main lumen (shaft 1020).

As described herein, since the nitinol tube 850 is fixedly attached to the knob and/or slider 822 (FIG. 16A) held within the handle, movement of the slider 822 causes axial movement of the nitinol tube 850, which in turn causes axial movement of the nose tip 860. As discussed herein, due to the present construction, the axial movement of the nose tip 860 facilitates either the expansion or contraction of the basket based on and in response to inflation or deflation of the balloon.

The nitinol tube 850 can thus be considered to be an actuation element for allowing and facilitating movement of the electrode baskets 870, 880 between the expanded state and the collapsed state.

The nitinol tube 850 also carries fluid flow into the balloon. The tube 850 is snugly fit inside of the braided polyimide tube 851, so there is not much of a gap for fluid flow. The majority of the flow goes inside the nitinol tube 850. In the handle 1010, there are holes laser cut in the nitinol tube 850 to allow fluid to flow inside, and in the basket end there are also holes laser cut into the tube for fluid to flow out of the tube and into the balloon.

As shown in FIG. 23, part of the design that allows for faster inflation speed is a step up in the inner diameter of the nitinol tube 850. The inner diameter (ID) needs to be smaller in the basket region so that the tube 850 has adequate wall thickness for adequate rigidity for supporting the basket. In the catheter main lumen (shaft 1020), the outer braided pebax tube provides the rigidity the catheter needs, so the nitinol tube 850 does not need as much wall thickness to provide rigidity. This is why it is safe to transition from one tube to another, through a tube welding process (FIG. 23 shows two tubes coupled to one another). The larger inner diameter (ID) for a majority of the length of the catheter allows for lower back pressure, resulting in higher flow rate and reduced balloon inflation time.

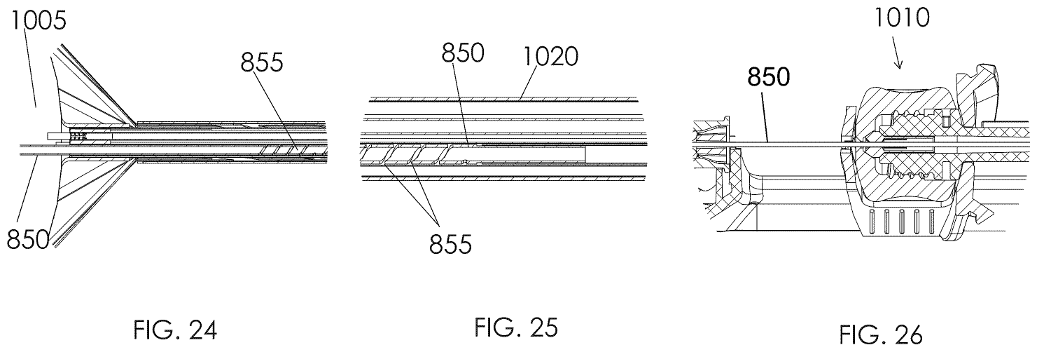

With reference to FIGS. 24-25, additional functionality that the nitinol tube 850 provides is a spiral cut section, generally shown at 855. This allows for improved bending flexibility in the region of the catheter that experiences bending in a deflectable sheath (that surrounds the catheter). This spiral cut section 855 also acts as a (distal) spring. When the balloon inflates, the balloon is expanded, and this pulls on the balloon, requiring the nose tip 860 to retract in a proximal direction. The nitinol tube 850 inside of the braided polyimide tube 851 has some resistance to motion, especially when the catheter is deflected in the sheath. There is also resistance to motion where the nitinol tube 850 goes through an O-ring in the handle 1010, where the O-ring is providing the seal and transition from fluid flow to outside air environment. As a result of all this resistance to motion, the balloon 1005 has to build a lot of pressure, which applies greater tensional/stretching force on the balloon, to finally overcome the resistance and move the nitinol tube 850 proximally. This results in the balloon 1005 bulging between the splines, which reduces the contact the splines have with the inside of the heart. This is where the nitinol spiral cut section 855 acting as a spring helps, by being closer to the movement in the basket, and compressing to absorb the motion. The long length of the spiral cut provides a low k factor (lbs/in, aka force per unit of deflection), which reduces the amount of pressure the fluid needs to provide to move the nitinol tube 850 proximally.

FIG. 24 illustrates that the spiral cut section 855 is located proximal to and outside of the balloon 1005 since it desirable for the portion of the tube 850 within the balloon 1005 to be rigid.

As mentioned, the spiral cut section 855 allows for movement of the tube 850 in the proximal direction since the spiral cut section 855 can compress due to its spring characteristics (i.e., the spacing between the windings are reduced as it compresses).

Since it is desired that the catheter remains in its collapsed state during delivery of the catheter to the target site, as typically occurs by passing it through an outer sheath device, a lock mechanism can be incorporated into the handle. The lock mechanism is desired to lock and prevent movement of the tube 850 which comprises the means for expanding the basket. Since one end of the tube 850 is attached to the (linearly) movable knob within the handle, the lock mechanism can be configured to lock the knob, and thereby lock the tube 850. The lock mechanism can be configured to lock the knob in the forward position which corresponds to the collapsed position of the basket. The lock mechanism can be a mechanical component that contacts/engages the knob or otherwise provides interference and prevents movement of the knob within the handle (e.g., prevents rearward movement of the knob in the handle). Once the catheter is delivered to the target position and it is time for inflation of the balloon, the lock mechanism is moved from the locked position to the unlocked position and this allows the knob to move rearwardly within the handle to accommodate the rearward movement of the tube 850 that results when the basket moves from the collapsed state to the expanded state.

When the tube 850 includes the spiral cut section 855, the handle 1110 does not include the spring 891 that is discussed previously herein with respect to a different embodiment (FIG. 16A).

### Locking Mechanism for Actuator Shaft

One of the challenges in constructing the balloon catheters described and illustrated herein concerns deployment of non-flexible polyimide splines (e.g., splines 875, 885) that deploy over a flexible urethane balloon 1005 (it will be appreciated that the balloon 1005 can be the same as balloon 830). A typical balloon would be fixed at both ends and stretch as needed to inflate to different shapes, but since the polyimide splines 875, 885 do not stretch, the present balloon catheter employes the axially movable (slidable) actuator shaft/tube 850 (e.g., the nitinol tube) that allows the distal end of the balloon catheter (e.g., the nose tip 860) to come proximal. The actuator shaft 850 will move on its own with enough force from the balloon 1005 inflating and pressing against the splines 875, 885, which pull on the distal nose tip 860, which is connected to the central actuator shaft 850. Since there is so much resistance to this actuator shaft 850 sliding throughout the catheter 1000 and from an O-ring that is disposed within the handle 1010, the splines 875, 885 must be under a lot of tension to cause the actuator shaft 850 to move, and this means they are pressing down on the balloon 1005 with all that force. This results in peaks and valleys in the balloon 1005, where the spline 875, 885 is creating the valley with the force it is pressing down on the balloon 1005. This results in poor contact with the inside of the heart (between the balloon catheter and the tissue).

Another problem, somewhat counter to the last problem, is that sliding of the central actuator shaft 850 proximally can cause the balloon 1005 to shift proximal, so that less of the balloon 1005 is in the endoscopes field of view, making it harder to confirm contact. When pressing the catheter against the vein, the balloon 1005 is being pushed in this direction, so the central actuator shaft 850 needs to hold the nose tip 860 forward, which holds the splines 875, 885 in place, and that holds the balloon 1005 in place.

Moreover, there are also some basic requirements of the actuator shaft 850. The actuator shaft 850 must be stiff in the basket region so that it remains straight and can withstand some sideways force as the user pushes the catheter in contact with the vein. The actuator shaft (nitinol tube) 850 must also bend just proximal of the basket, so that the sheath is able to bend it towards hard-to-reach veins.

The actuator shaft (e.g., nitinol tube) 850 is also designed to have some springiness which has multiple benefits. The nitinol tube has some compliance to move with force, so the adjustment of the knob 1151 in the handle 1010 does not need to be perfectly located to get good deployment of splines 875, 885 over the balloon 1005 (this is thus a balance of opposing problems previously discussed above).

Moreover, as the heart is moving, and especially as PFA energy is being delivered and the heart convulses, the surgeon will see the vein press up against the balloon 1005 more, and sees the nitinol tube 850 piston back and forth from this additional force. A spring, such as those described herein, provides some compliance to this force and ensures better contact with the vein throughout the movement. This can also be a safety benefit in allowing the nitinol tube movement to absorb the vein movement, instead of holding place and resisting that motion with a lot of force. This is especially important in PFA as the energy delivery can cause the patient to cough, some patients more than others, and this results in the same heart convulsing.

Based on the foregoing, there is a need for a solution in which the user is provided with some control of this sliding nitinol tube movement, and/or for the moving pieces to slide on their own. Proper design of the mechanism is critical to balancing the two opposing problems.

As previously discussed, to insert the catheter through the sheath, the surgeon needs to collapse the balloon 1005 by pushing the actuator shaft (nitinol tube) 850 forward (i.e., move in the distal direction) and lock this forward position of the nitinol tube 850 in the handle 1010. Once again, this forward position of the nitinol tube 850 corresponds to the spline basket being in the collapsed state. By locking the nitinol tube 850 in this forward position, the basket is maintained in the collapsed state and the nitinol tube 850 is prevented from moving in the proximal direction which would result in an undesired expansion of the basket. As described herein, there are several different lock mechanisms that can be used to ensure that the nitinol tube 850 is locked in the axial (longitudinal) direction.

### Knob Ratchet Mechanism (FIGS. 27-31)

FIGS. 27-31 illustrate one locking mechanism 1300.

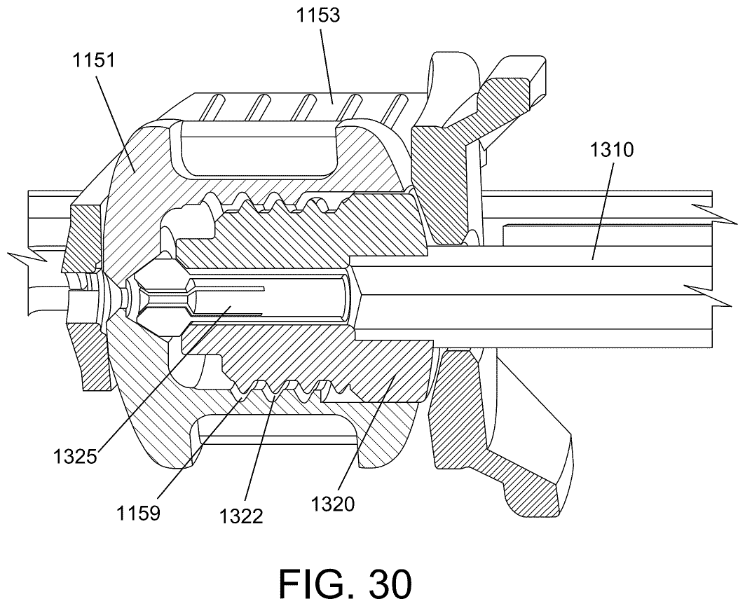

The locking mechanism 1300 includes a first lock shaft 1310 that has a proximal end and a distal end. At the distal end, there is a coupling member 1320 that can have a cylindrical shape and is disposed within the hollow interior of the knob 1151. As shown in FIG. 30, the coupling member 1320 can have a contoured outer surface for engaging the inside of the knob 1151 resulting in a coupling between the first lock shaft 1310 and the knob 1151. For example, as shown, the outer surface of the coupling member 1320 has first threads 1322 that are formed in a longitudinal direction and are spaced apart from one another. The first threads 1322 engage and interlock with complementary second threads 1159 that are formed on the inside of the knob 1151 to allow the knob 1151 to be screwed onto the coupling member 1320. Within the coupling member 1320 there is also another part 1325 that is configured to grasp and hold the actuation tube 850 (FIG. 16B). As a result, when the first lock shaft 1310 moves in an axial (longitudinal) direction, the actuation tube 850 also moves axially and similarly, when the first lock shaft 1310 rotates, the actuation tube 850 rotates. As mentioned since the knob 1151 is carried by the slider 1153, axial sliding of the slider 1153 by the user is directly translated into axial movement of the first lock shaft 1310 and the actuation tube 850. In addition, rotation of the knob 1151 is translated into rotation of the first lock shaft 1310.

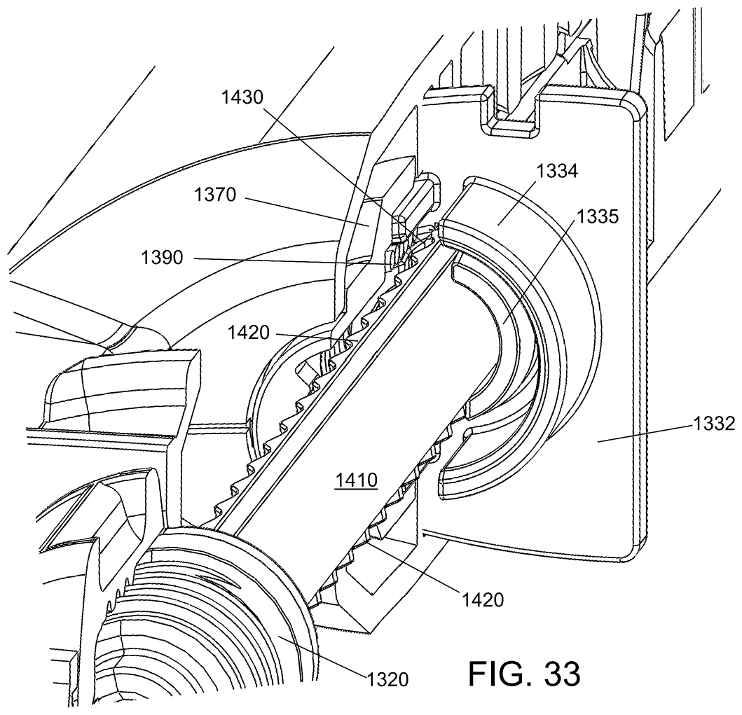

As shown, the first lock shaft 1310 can be a tubular structure with a hollow center lumen and a cylindrical shape. The first lock shaft 1310 also includes a pair of longitudinal rails or fins 1314 that are formed along the outside of the first lock shaft 1310 can extend in the longitudinal direction. In the illustrated embodiment, there are two fins 1314 that are located 180 degrees apart. Along one surface of each fin 1314 there are a series of rounded teeth 1315 that extend along the length of the fin 1314. Unlike the embodiment of FIG. 33 which utilizes sharp teeth, the teeth 1315 have rounded, smooth edges (rounded peaks and rounded valleys between the rounded peaks). As will be appreciated in the discussion below, the teeth 1315 can be considered to be a rack of teeth and the rounded design allows for axial slip of the teeth 1315 when a sufficient axial force is applied.

As shown, the rounded teeth 1315 of one fin face outward in a first direction and the rounded teeth 1315 of the other fin face outward in a second direction opposite the first direction.

As shown, the fins 1314 do not need to extend the entire length of the first lock shaft 1310 and can extend to the proximal end of the first lock shaft 1310 but not all the way to the distal end of the first lock shaft 1310.

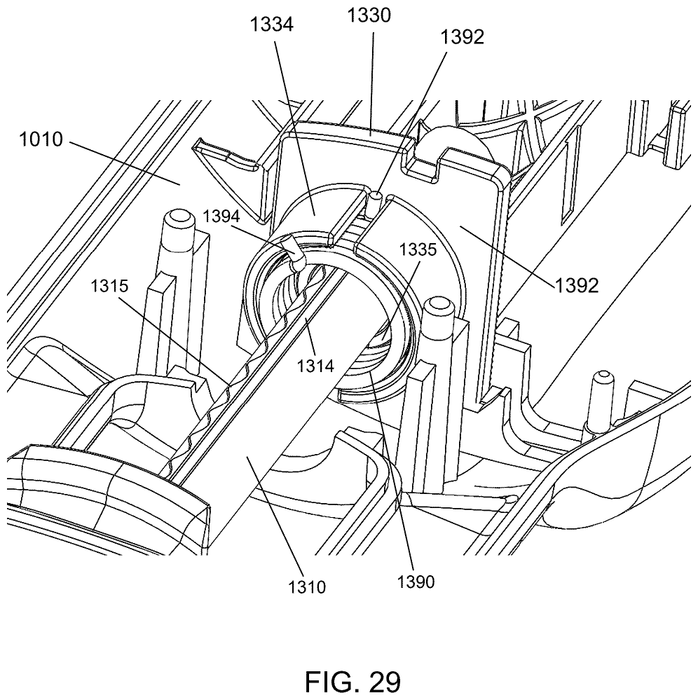

The locking mechanism 1300 also includes a locking plate 1330 that is fixedly disposed within the handle 1010. As best shown in FIG. 29, the locking plate 1330 is an upstanding structure that includes a rear base 1332 and an outer split ring protrusion 1334 that is integral with the rear base 1332 and extends outwardly therefrom (such as at a 90 degree angle) along with an inner split ring protrusion 1335 that is integral with the rear base 1332 and extends outwardly therefrom (such as at a 90 degree angle). The outer split ring protrusion 1334 is located radially beyond (outside) of the inner split ring protrusion 1335.

When assembled within the handle 1010, the outer split ring protrusion 1334 and the inner split ring protrusion 1335 extend in the distal direction toward the knob 1151 and the slider 1153. The illustrated outer split ring protrusion 1334 can be considered to be an outer boss and comprises a pair of discontinuous arcuate shaped walls with a pair of gaps 1336 between the ends of the arcuate shaped walls. Similarly, the illustrated inner split ring protrusion 1335 can be considered to be an inner boss and comprises a pair of discontinuous arcuate shaped walls with a pair of gaps 1337 between the ends of the arcuate shaped walls. The gaps 1336 and 1337 are at least partially aligned with one another.

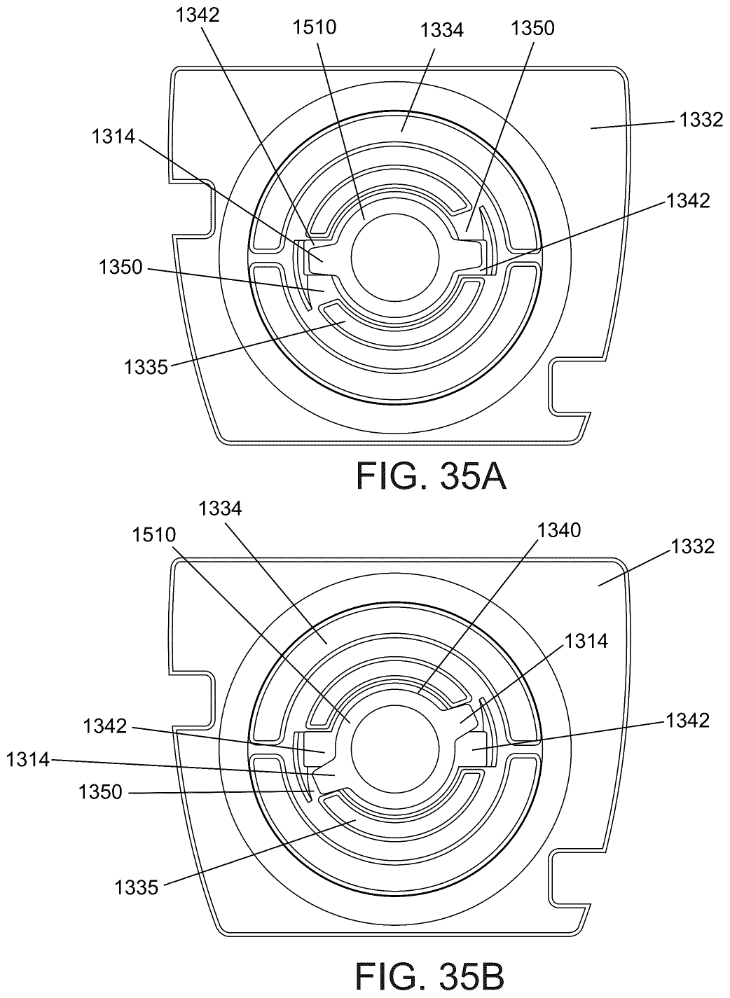

Inside of the inner split ring protrusion 1335 is a keyed center opening 1340 that is best illustrated in FIGS. 35A and 35B. The keyed center opening 1340 has a center hole that passes through the rear base 1332 and a pair of keyed slots 1342 that extend outward from the center hole and as shown, can be oriented 180 degrees apart. The keyed slots 1342 are sized to allow passage of and axial movement of the fins within the keyed slots 1342.

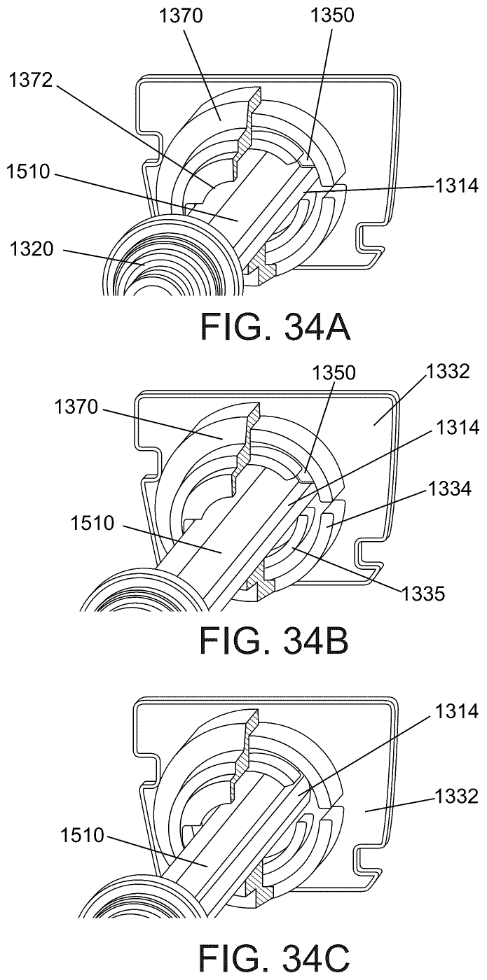

As shown in FIGS. 35A and 35B, one end of the arcuate shaped wall of the inner split ring protrusion 1335 is proximate to and can abut one edge of one keyed slot 1342; however, the other end of the arcuate shaped wall does not extend to the other edge of the one keyed slot 1342, thereby creating a locking space 1350 defined between the other end of the arcuate shaped wall and the one keyed slot 1342 and located in front of the rear base 1332. The functioning of this locking space 1350 is described in more detail with respect to the other embodiment illustrated in FIGS. 34A-34C.

One end of each of the arcuate shaped segments of the inner split ring protrusion 1335 is contoured and has a surface profile that is complementary to the rack of rounded teeth 1315. In particular, the one end of each of the arcuate shaped segment has an undulating locking surface 1360 that faces into the gap 1337. The undulating locking surface 1360 is complementary to the rounded teeth 1315 in that in one position between the two, the rounded teeth 1315 sit within the rounded valleys of the undulating locking surface 1360. This position can be considered to be a locked or engaged position since relative axial movement between the first lock shaft 1310 and the locking plate 1330 is restricted. However, this locking between the rounded teeth 1315 and the undulating locking surface 1360 can be considered to be more of a holding force since it can be overcome by pushing the knob 1151 forward or backwards with enough force. In other words, when sufficient force is applied in the axial direction, the rounded teeth 1315 will slip out of engagement with the undulating locking surface 1360 and then reengages with the undulating locking surface 1360. This slippage action and the reengagement is assisted by a biasing action described below.

It will be appreciated that since the fins 1314 are oriented 180 degrees apart, the first lock shaft 1310 is held and engages the locking plate at two locations 180 degrees apart.

The locking mechanism 1300 also includes a biasing mechanism that applies a biasing force to the first lock shaft 1310 to assist in maintaining it in the held position but permit axial movement under sufficient axial applied force. The biasing mechanism comprises a torque translation cap 1370 and a torsion spring 1390. The torque translation cap 1370 is configured to fit over the locking plate 1330 and rotate relative to the locking plate 1330. The torque translation cap 1370 does not move axially and is only capable of rotation.

The torque translation cap 1370 is a hollow part that has a rear end that seats against the locking plate 1330 and an opposite front end. The front end of the torque translation cap 1370 has an opening 1372 (FIG. 34A) through which the first lock shaft 1310 passes. The construction of the torque translation cap 1370 is such that the first lock shaft 1310 can moved axially relative to the torque translation cap 1370 by moving axially within and through the opening 1372; however, the first lock shaft 1310 cannot rotate relative to the torque translation cap 1370. Instead, the first lock shaft 1310 and the torque translation cap 1370 rotate together. This can be achieved by having guide slots and/or guide features in the torque translation cap 1370 that receive the fins to permit axial movement of the first lock shaft 1310 but prevent any rotation of the first lock shaft 1310 relative to the torque translation cap 1370.

The torsion spring 1390 is best shown in FIG. 29. As is known, the torsion spring 1390 is a type of mechanical spring that works by exerting torque or twisting force when it is twisted along its axis. The torsion spring 1390 is made of wire that is wound in a spiral shape, with first and second ends 1392, 1394 of the wire attached to a stationary point on one end and a rotating point on the other end. In the present application, the first end 1392 is received within the first gap 1336 that is part of the outer split ring protrusion 1334 and thus represents the end of the torsion spring 1390 that is fixed. The second end 1394 of the torsion spring 1390 is the end that is fixed to a rotating point on account of the second end 1394 being attached to the rotatable torque translation cap 1370. For example, inside of the torque translation cap 1370 there is a structure that allows for attachment of the second end 1394. For example, inside of the torque translation cap 1370 there can be at least two securement tabs 1375 with a space between the two tabs 1375 into which the second end 1394 can be received to thereby fix the second end 1394 to the torque translation cap 1370. Since the torque translation cap 1370 rotates relative to the locking plate 1330, the torsion spring stores and release energy. Since the first lock shaft 1310 is coupled to the torque translation cap 1370, the torque action of the torque translation cap 1370 is imparted to the first lock shaft 1310.

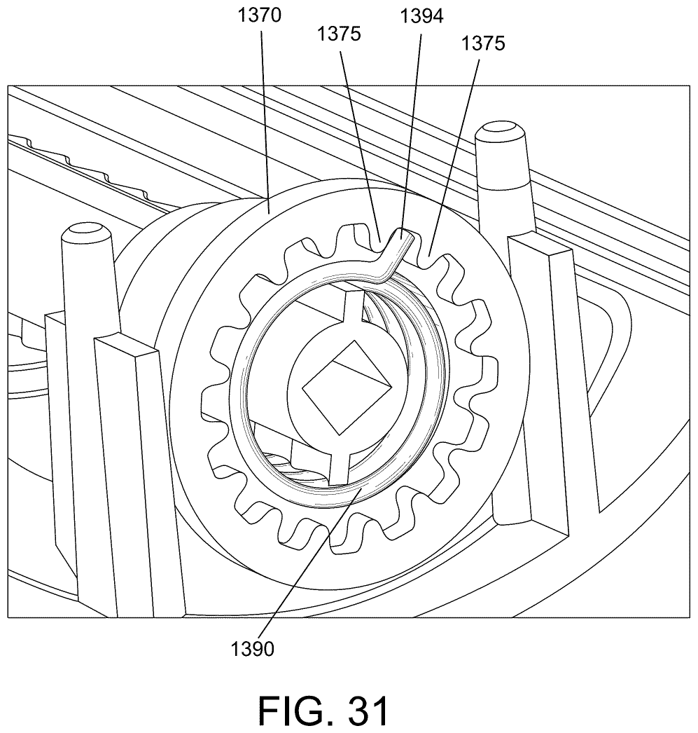

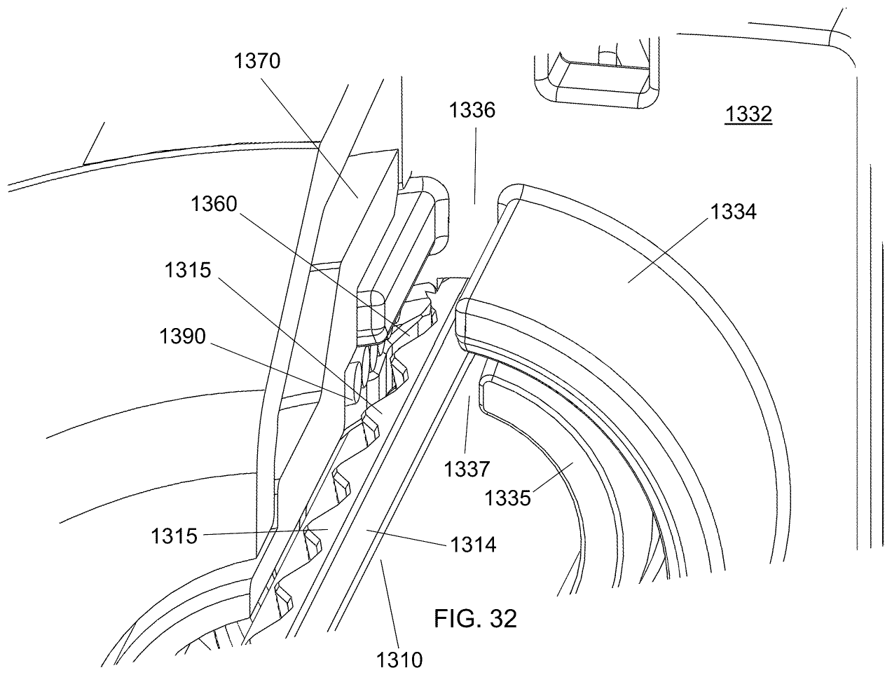

FIG. 31 shows securement tabs 1375 extending around the complete inner circumference of the torque translation cap 1370; however, it will be appreciated that the tabs 1375 do not have to extend around the complete inner circumference.

The lock mechanism 1300 operates in the following manner. The torsion spring 1390 is always under tension and pushes off of the locking plate 1330 and acts on the torque translating cap 1370. More specifically, the stored energy of the torsion spring 1390 acts on the first lock shaft 1310 in a counterclockwise direction in that the first lock shaft 1310 wants to rotate in the counterclockwise direction due to the action of the torsion spring 1390. This torsion spring force will cause the rounded teeth 1315 to be pulled into engagement with the undulating locking surface 1360 resulting in the first lock shaft 1310 being held in a given axial position. However, as mentioned, this holding force can be overcome by applying sufficient axial force as by pushing or pulling the knob 1151 in the axial direction. Under this axial force, the rounded teeth 1315 will slip out of engagement with the undulating locking surface 1360 and momentarily the peaks of the rounded teeth 1315 will be seated against the peaks of the undulating locking surface 1360; however, the stored tension in the torsion spring 1390 continues to apply a counter clockwise rotational force to the first lock shaft 1310 (by means of the torque translation cap 1370) and thus, as the first lock shaft 1310 advances and the peaks of the rounded teeth 1315 are placed into alignment with the valleys of the undulating locking surface 1360, the torsion spring pulls the peaks of the rounded teeth 1315 into engagement with the valleys, thereby locking or holding the first lock shaft 1310 in this axially advanced position. This advancement is akin to a ratcheting action. In other words, during the movement, the first lock shaft 1310 rotates slightly as the axial force translates into torsion and the torsion from the torsion spring is overcome so that the rounded teeth 1315 can climb up and over to the next slot. The smooth tooth design easily and consistently translates axial force into torsional force.

As previously mentioned, during delivery of the catheter 1000, the splines 875, 885 need to remain in the collapsed state and the lock mechanism 1300 achieves this objective by allowing the user to use the slider 1153 or knob 1151 to axially advance the first lock shaft 1310 to the forward (most distal) position which corresponds to the nose tip 860 being in its most distal position and the splines are in the collapsed state. The lock mechanism 1300 ensures that the first lock shaft 1310 and thus, the nitinol tube 850 remain in this most distal position during delivery of the catheter. Once at the site and deployment of the spline basket is desired, the first lock shaft 1310 is ratcheted in the opposite proximal direction cause the nitinol tube 850 and nose tip 860 to move proximally, thereby allowing expansion of the splines 875, 885 to the expanded position. In this position, the lock mechanism 1300 will permit maintenance of the splines in the expanded position since the nitinol tube 850 and nose tip 860 are maintained in a locked (held) position. The torsion spring ensures such various axial positions are held during use of the catheter.

### Knob Lock Mechanism (FIG. 33)

FIG. 33 illustrates an alternative lock mechanism 1400.

The lock mechanism 1400 shares similarities to the lock mechanism 1300 and therefore, like elements are numbered alike. The main difference is in the construction of a second lock shaft 1410 that, like the first lock shaft 1310, has a pair of fins. However, instead of using rounded teeth 1315 that mesh and engage with the undulating locking surface 1360, the second lock shaft 1410 includes a series of sharper teeth 1420 that mesh with complementary teeth 1430 that are part of the locking plate. This design does not allow the slippage that the first lock shaft 1310 exhibited but instead the forward and backward advancement of the second lock shaft 1410 requires that the second lock shaft 1410 be first rotated in a clockwise direction to disengage the teeth 1420, 1430. The rotation of the second lock shaft 1410 results from rotation of the knob 1151 due to the direct connection between the two.

Once the teeth 1420 and teeth 1430 are disengaged, the second lock shaft 1410 can be freely advanced in the axial direction to a more distal or proximal position. Once the desired position is reached, the user simply releases the knob 1151 and the stored energy of the torsion spring 1390 automatically causes the counterclockwise rotation of the second lock shaft 1410 which results in the teeth 1420, 1430 reengaging into a lock position.

In this way, the second lock shaft 1410 can be advanced and held and locked in any number of different axial positions. The second lock shaft 1410 functions similar to a spring-loaded pawl that engages teeth on a rack.

### Knob Lock Forward Mechanism (FIGS. 34A-35B).

FIGS. 34A to 35B depict another alternative lock mechanism 1500 that is similar to the other lock mechanisms 1300, 1400 and therefore, like elements are numbered alike. In this embodiment, the lock mechanism 1500 includes a third lock shaft 1510.

In the third lock shaft 1510, the two fins 1314 are unadorned in that they lack any teeth and are smooth. As with the other embodiments, the torsion spring 1390 and the torque translating cap 1370 always act on the third lock shaft 1510 and translate a rotational force onto the third lock shaft 1510.

FIG. 34A shows an unlocked position of the third lock shaft 1510. The fins 1314 are within the keyed slots 1342 formed in the rear base 1332 and thus, the third lock shaft 1510 can freely move in the axial direction. Since the third lock shaft 1510 moves axially relative to the torque translating cap 1370, the third lock shaft 1510 can freely move axially in this position. In FIG. 34B, the third lock shaft 1510 has moved forward (distally) such that the proximal ends of the fins 1314 have cleared the keyed slots 1342. Once the proximal ends of the fins 1314 clear the keyed slots 1342, the interference between the third lock shaft 1510 and the rear base 1332 is removed and therefore, the torque action of the torsion spring 1390 and torque translating cap 1370 causes the automatic counterclockwise rotation of the third lock shaft 1510. This results in the proximal ends of the fins 1314 moving into the locking spaces 1350 as shown in FIGS. 34C and 34B. Once the fins 1314 occupy the locking spaces 1350, the fins 1314 abut the rear base 1332 and thus, movement of the third lock shaft 1510 in the proximal direction is prevented which ensures that the spline basket remains collapsed.

The lengths of the fins 1314 are purposely created such that when the proximal ends thereof clear the keyed slots 1342, the third lock shaft 1510 (and the nitinol tube 850 and nose tip 860) is in the forward (most distal) position that corresponds to the spline basket being in the collapsed state.

To unlock the third lock shaft 1510, the knob 1151 is rotated to cause clockwise rotation of the third lock shaft 1510 to position the fins 1314 into the keyed slots 1342 as shown in FIG. 35A. Once there is registration between the fins 1314 and the keyed slots 1342, the third lock shaft 1510 can be moved axially in the proximal direction to allow the spline basket to move to the expanded state.

In yet another embodiment, the catheter can be constructed such that a spring is incorporated into the inside of the basket/balloon. Like the previous nitinol spring design (FIG. 25), this design allows for the nitinol tube 850 to move forward and back without the knob 1151 in the handle 1010 moving. The shaft in the balloon basket is split into two pieces which slide axially with respect to one another, where one method of achieving this is axial motion is a nitinol rod that slides inside of a nitinol tube. This motion is linked by a spring that resists the rod sliding into the tube. This design functions similar to the other nitinol spring designs where there is resistance to the proximal movement of the distal end of the catheter. This resistance is tuned to balance the opposing problems described herein.

### Collapsed and Expanded Positions

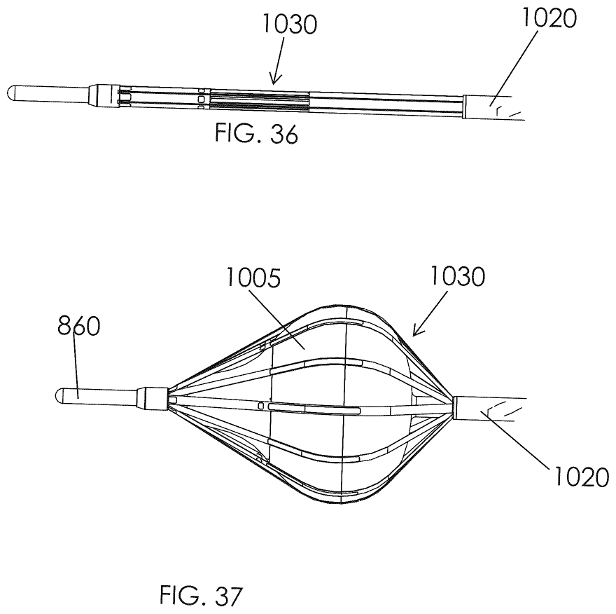

FIG. 36 illustrates the catheter 1000 in a collapsed position. In this collapsed position, the balloon and expandable basket are collapsed and this allows for insertion of the catheter 1000 into a surrounding device, such as a deflectable outer sheath (not shown).

FIG. 37 illustrates the catheter 1000 in an expanded position, whereby both the balloon and the expandable baskets 870, 880 are expanded.

### Electrode Marker

One challenge of using a balloon catheter that includes splines and electrodes located along the splines is determining the orientation of the balloon, and thus electrodes, in the heart. As described herein, the user (operator) is looking through the endoscope image from inside the balloon, which is inside the heart, as well as with fluoroscopy and impedance-based 3D mapping systems. All of these systems need a way of identifying the orientation of the balloon, and ways of identifying the orientation with respect to the other visualization methods. For example, it can be challenging to understand from the endoscope image what the orientation is, but if the user can easily locate electrode #1 on the endoscope image, and the user can locate electrode #1 on the 3D mapping system, then the user can understand the orientation from the mapping system and then use that to understand the orientation in the endoscope view. The same relationship is needed with the fluoroscopy image with the other methods described. Knowing the orientation is important for tailoring the balloon contact, as well as possibly tailoring the ablation parameters for all the splines or for specific splines.

ICE is another visualization method but does not have the same needs for identification of orientation. Other mapping systems such as Luma Vision 3D ICE are being developed and typically use a magnetic sensor on the catheter to establish orientation.

Accordingly, the endoscopically guided catheter ablation process involves looking at the endoscope image and using it to know when you have good contact with the inside of the pulmonary vein. The confirmation of contact guides the doctor. One aspect of this endoscope view that is important is seeing the location of the flex strips (that carry the electrodes). Since the dark polyimide flex strips do not reflect much of the light being shot inside the balloon, Applicant uses a white marker on the backside of the splines (flex strips) to reflect the light. The white marker shows the extent of the electrode, so the user can easily see where the electrode 1100 is located with respect to the contact that is observed, which is shown by white areas (e.g., the length of the white marker on the backside of the spline corresponds to the length of the physical electrode). In an endoscopic image, the dark areas are where there is blood around the balloon, which is where there is no contact with the inside of the heart.

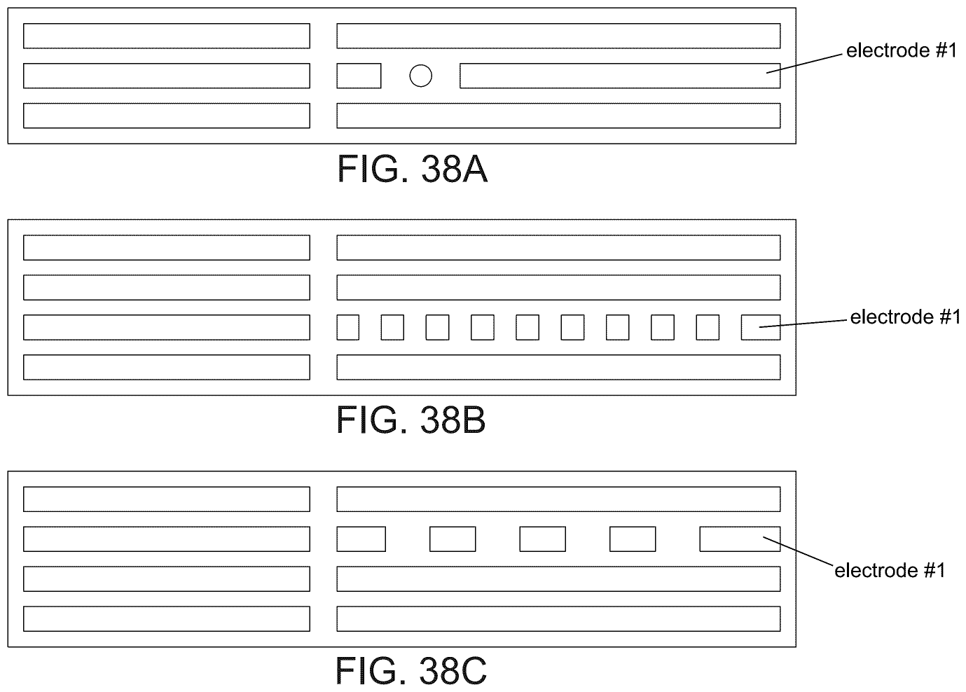

As mentioned, and discussed herein, one aspect that's helpful in orienting the doctor is knowing which electrode is which when viewing the endoscopic image at the operative site. To achieve this objective, one reference electrode is labeled as electrode #1. One method is to visually differentiate the electrode #1 relative to the other electrodes. For example, a unique ink pattern is used to mark electrode #1. In FIG. 38A, the electrode #1 is shown having a circular dot that interrupts the marked region that defines the length of the electrode. Thus, electrode #1 is easily identified in the endoscopic image due to its different looking white marker compared to the others which are simply uninterrupted white markers.

The location of all the other electrodes can be determined by counting clockwise from electrode 1, as the splines are numbered sequentially going clockwise 1 through 12. For many applications, it is not important to know where every individual spline is, but just knowing which electrode is #1 helps to align what the doctor sees in the 3D mapping software with what they're seeing in the endoscope view.

There is also the possibility of individually ablating between specific electrodes, and knowledge of which spline is which number, as well as seeing the contact around those splines, helps the doctor to apply a more tailored ablation parameters for that situation. There could also be overcurrent or sparking happening in one area, and also the doctor could recognize that situation happening beforehand by seeing the splines too close to each other. Either scenario could lead to the doctor deciding to choose certain electrodes to ablate across, deciding to skip 1 or 2 splines, for example ablating between splines 1 and 4, instead of the typical ablating between splines 1 and 3.

In a different embodiment, the marker can constitute two markers, such as bands, that mark the two ends of the electrode. Between the two band markers, there is no marker and thus, if the two bands are visible in the endoscopic view, the user can determine the location of the electrode.

FIG. 38B shows another embodiment in which the electrode #1 is shown having a series of spaced apart square markers that interrupt the marked region that defines the length of the electrode. Thus, electrode #1 is easily identified in the endoscopic image due to its different looking white marker compared to the others which are simply uninterrupted white markers.

FIG. 38C is similar to FIG. 38B but includes a series of longer spaced apart rectangular markers.

Thus, the use of radiopaque ink that is visible in fluoroscope can be used to determine a reference electrode, in this case electrode #1, and once the location of the electrode #1 is determined, the locations of the electrodes can be determined. However, there is one challenge. FIG. 39A is a sample fluoroscopy image and the radiopaque marker identifying spline #1/electrode #1 is generally shown at 990 (which is visually different than the markers of the splines/electrodes). It will be understood that in each of the embodiments described herein, the radiopaque marker (radiopaque markers) can be formed along the inner surface of the spline that faces the balloon or the radiopaque marker can be formed along the outer surface of the spline that faces away from the balloon so long as the radiopaque material does not overlap with the electrode(s) on said spline. The radiopaque marker is visible on a fluoroscopy image whether it is formed along the inner spline surface or outer spline surface.

However, the user cannot tell whether the electrode #1 is closest to the viewer or whether the electrode #1 is on the far side of the balloon. To counter this deficiency, the electrodes are designed so that in addition to the special marker 990 for electrode #1, there are two other reference electrode markers that are positioned at known angles and locations relative to the electrode #1. For example, there can be a first reference electrode marker 991 that is separated from the electrode #1 by 90 degrees and the second reference electrode marker 992 that is also separated from the electrode #1 by 90 degrees in the opposite direction.

The first and second reference electrode markers 991, 992 are visually different from the electrode marker 990 for the electrode #1. In the illustrated embodiment, the electrode marker 990 for electrode #1 is longer in length than the first and second reference electrode markers 991, 992. To further differentiate, the electrode marker 990 for electrode #1 from the first and second reference electrode markers 991, 992, the first reference electrode marker 991 is oriented (formed) distal to the electrode marker 990 and the second reference electrode marker 990 is oriented (formed) proximal to the electrode marker 990.

In FIG. 39B, the user can see the bottom right radiopaque marker (i.e., the second reference electrode marker 992) is visible, so the user knows that electrode #1 must be on top of the balloon (coming out of the page toward the reader), since it is only 90 degrees offset from electrode #1 in the fluoroscopy image. If electrode #1 was on the far side of the balloon (i.e., facing into the page away from the reader), then the second reference electrode marker 992 would have to be 180 degrees offset from the marker 990 of electrode #1. In FIG. 39B, the other marker in the top left represents the first reference electrode marker 991 and is not needed in this orientation and is the less useful of the two in this orientation of balloon, but in scenarios where the bottom right marker (second reference electrode marker 992) is in line with the electrode #1, or otherwise hard to distinguish, having a second marker on the other side makes sure that there is always one electrode marker within view to easily identify orientation of the balloon and the electrodes. In other words, the first and second reference electrode markers 991, 992 allow the user to understand with certainty the location of the electrode #1 using the fluoroscopy image.

As mentioned, the first and second reference electrode markers 991, 992 are visually identifiable from the electrode marker 990 of the electrode #1. For example, the first and second reference electrode markers 991, 992 can have a different graphical pattern (e.g., shape, length, etc.) and further, as discussed, they can be distal and proximal to the electrode marker 990 of the electrode #1. In the illustrated embodiment, the marker lengths and the proximal and distal locations visually identify the first and second reference electrode markers 991, 992 relative to the electrode marker 990 of the electrode #1.

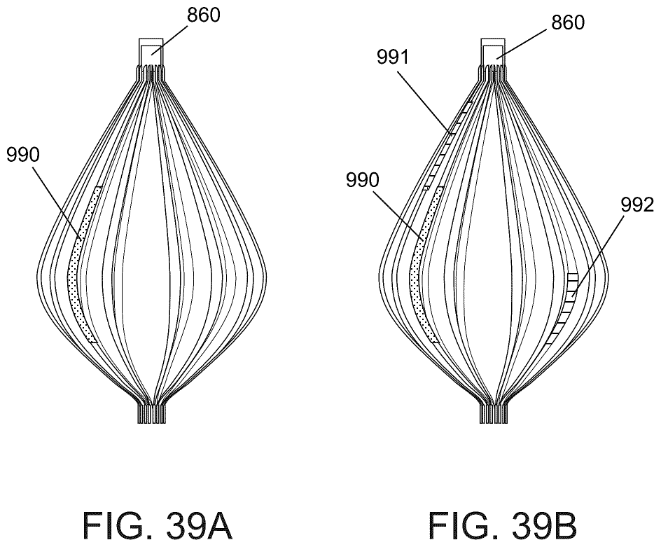

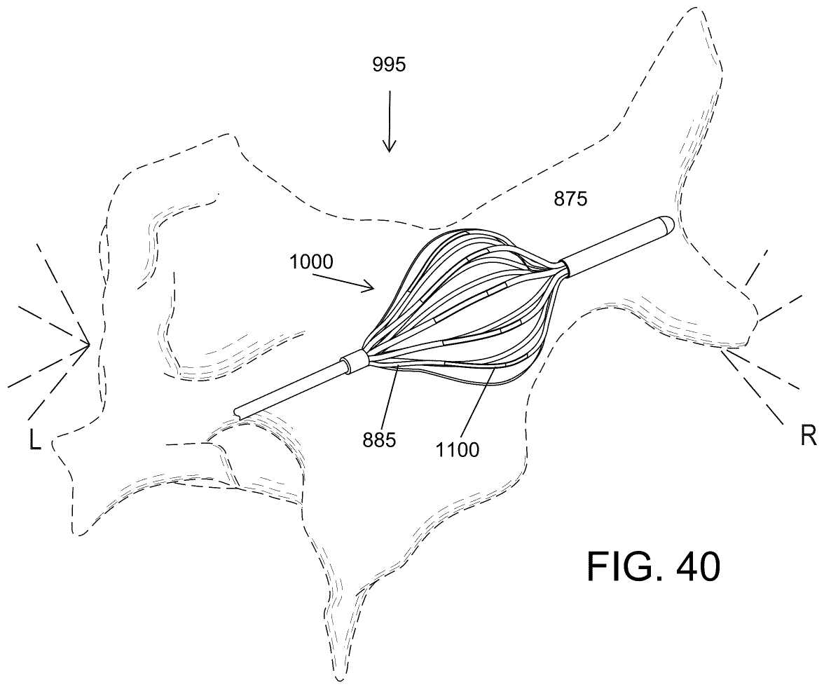

Now referring to FIG. 40 in which a 3D mapping image 995 is shown. As mentioned herein, a 3D mapping system is a medical technology that uses 3D mapping to help electrophysiologists navigate the heart and locate catheters accurately during diagnosis and treatment of cardiac arrhythmias, as well as during other treatments. This system generates real-time 3D maps by processing local electrograms and spatial information from the catheter tip (use of electromagnetic technology). The system uses a triangulation algorithm, similar to GPS, to precisely localize the catheter. These systems also offer other graphical cues, including graphical representations, that allow the user to determine the location of and/or direction toward the patient's head to allow the user to better understand the orientation of the displayed 3D mapping image.

In the 3D mapping image of FIG. 40, anatomical features of the heart are shown. The catheter 1000 is also shown. In addition to understanding the position of the catheter 1000, the splines 875, 885 and electrodes 1100 can also be seen. In one embodiment, the electrode #1 can be graphically highlighted on the 3D mapping video feed in a manner that is visually different than the graphic representation of the other splines.

Thus, in one embodiment, the user is provided with multiple imaging tools that show the catheter 1000 and electrodes 1100 at the surgical site and more particular, the user will have: 1) the real-time endoscopic view; 2) fluoroscopy images; and 3) 3D mapping images. For example, on a display screen, the live endoscopic view can be positioned on one side of the display (e.g., left-side) and the real-time 3D mapping image can be positioned on another side of the display (e.g., right-side).

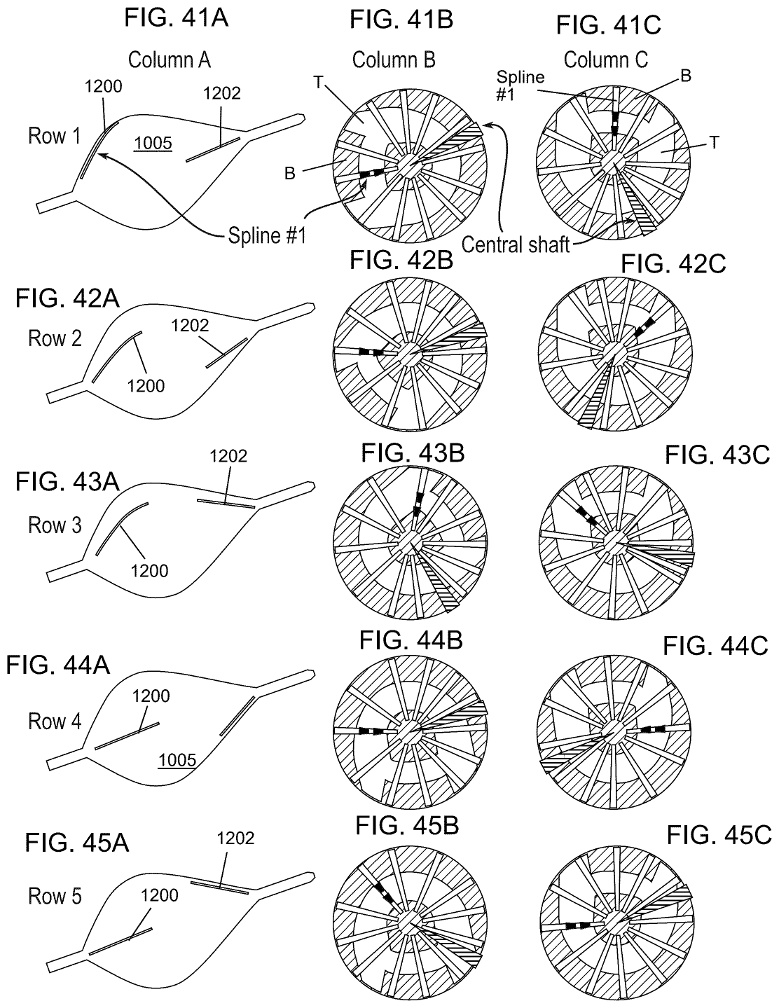

### Pulmonary Vein Application (FIGS. 41A to 45C)

As previously mentioned, and according to one application, a balloon catheter with an endoscope inside the ballon is positioned so the balloon is in contact with the antrum of a pulmonary vein in order to apply pulsed electrical field energy to the vein to treat atrial fibrillation. Wrapped around the balloon 1005 are twelve (12) longitudinal splines 875, 885 with electrodes 1100 on their outer facing surfaces which deliver the PFA energy and which are ordinarily not visible under fluoroscopy but may be made radiopaque by known means such as applying radiopaque ink, metallic film or other radiopaque to the material as described herein. The endoscope views the inside of the transparent balloon looking toward the distal tip of the balloon 1005. The field of view of the endoscope is such that the entire distal surface of the balloon 1005 is visible from the maximum diameter of the balloon forward. This area comprises the part of the balloon that generally contacts the vein antrum during an ablation procedure. Where vein tissue contacts a portion of the balloon 1005 that portion appears light colored in the endoscopic image. In FIGS. 41B-45C, the tissue contact is identified by reference character “T”. Where no tissue is in contact with a portion of the balloon, such as the portion of the balloon located in the lumen of a pulmonary vein or the lumen of an adjacent vein or in the atrial chamber, red blood is seen on that portion of the balloon in the endoscopic image. In FIGS. 41B-45C, the blood contact is identified by reference character “B”.

The twelve longitudinal splines 875, 885 are in contact with the balloon 1005 and are marked with white on their inner facing surfaces and are hence also visible to the endoscope. On the outer surfaces of these splines 875, 885 are located the electrodes 1100 that deliver the PFA energy to atrial tissue. One of the splines 875, 885 is designated as spline #1 and is marked with two black marks visible to the endoscope so as to be visually distinguishable from the other splines. The white splines are visible in the endoscopic view as radial white lines. It will be appreciated that other marks, such as the ones described herein, can be used instead of the two marks in the figures (FIGS. 41B and 41C).

When the balloon catheter is positioned in the vein, the rotation of the catheter 1000 and the rotation of the endoscope inside the catheter have no particular relationship to the patient's anatomy. Hence, as mentioned herein, the physician user is not able to determine by looking at the endoscopic image alone which direction is superior, (i.e. toward the patient's head) which direction is anterior, inferior, etc. Such information is important to the physician since adjusting the position of the balloon catheter in the pulmonary vein to optimize contact between the vein and the ballon is dependent upon the physician knowing in which direction the catheter should be pushed or deflected to optimize balloon contact with the vein.

The problem to be solved then is one of how to reorient the initially randomly oriented endoscopic image such that the upper most part of the reoriented image is toward the patient's head.

The catheters 1000 and systems described here solve the problem of knowing how to correctly adjust the endoscopic image orientation by marker at least two of the splines 875, 885 with radiopaque material that renders them visible under fluoroscopy so that the rotational orientation of the balloon 1005 relative to the patient's anatomy can be determined using a fluoroscopic image of the balloon while the balloon is positioned in the patient; and further providing a marker on at least one of the radiopaque splines 875, 885, said marker being visible to the endoscope in the balloon so that the orientation of the endoscope image may be adjusted to match the rotational orientation of the balloon as determined by the fluoroscopic image.

FIGS. 41A to 45C present five rows of images. The first image in each row is a representation of how the balloon with radiopaque splines appears in a fluoroscopic image of the balloon 1005 while in the patient. Such fluoroscopic images are typically produced such that the direction of the patient's head is toward the top of the fluoroscopic image. As mentioned, two of the splines (e.g., splines 875, 885) on the balloon 1005 have been rendered radiopaque (e.g., using radiopaque ink) along separate portions of the spline's respective lengths. In this embodiment, one spline has been made radiopaque along the proximal half of its length by using a first radiopaque marker 1200 and a second spline has been made radiopaque along the approximately distal half of its length by using a second radiopaque marker 1202. Importantly the two marked splines are oriented a known angular distance apart from one another, in this case 90 degrees to one another with respect to their circumferential orientation around the balloon. In other words, the first radiopaque marker 1200 and the second radiopaque marker 1202 are oriented 90 degrees apart along with being separated in the proximal-distal direction.

As mentioned previously, the first radiopaque marker 1200 and the second radiopaque marker 1202 can be formed either on the outer surface of the respective splines so long as the radiopaque material does not overlap the electrode(s) formed along the outer surface of each of the splines. Alternatively, the radiopaque marker can be formed along the inner surface of each of the splines. As described, herein in the case that the radiopaque marker is on the inner surface of the spline, the radiopaque material would be applied to the spline first. Then, a white ink (“paint) to designate the location and longitudinal extent of each of the electrodes (under endoscopic imaging) would be applied to the inner surface of the spline and this ink would be applied on top of the radiopaque material on the two splines that have said radiopaque material (markers 1200, 1202) on them. Then, an additional visual identifier or identifiers that can be viewed in the endoscopic image, such as one or more graphic mark, such as the two black marks on spline #1 described below, would be applied on top of the white painted section(s).

For purposes of illustration, in FIGS. 41A, 42A, 43A, 44A, 45A, the two radiopaque markers 1200, 1202 are located along the outer surfaces of the two corresponding splines and are at locations that do not overlap the electrode(s) on the respective spline.

If an observer is at the proximal most end of the balloon 1005 and is looking forward toward the distal end of the balloon 1005 and the spline that has its proximal section rendered radiopaque (i.e., identified by the first radiopaque marker 1200) is at the 12 o'clock position, then the spline that has it's distal section rendered radiopaque (i.e., the second radiopaque marker 1202) would be at the 3 o'clock position. With the radiopaque splines so orientated relative to one another on the balloon, their visual appearance in a profile view of the balloon (the profile view is the usual view when observing the balloon fluoroscopically during an ablation procedure (e.g., See, FIG. 41A)) is unique for any rotation of the balloon. This unique appearance of the radiopaque splines will be described in more detail below.

Additionally, and importantly, spline #1 (the spline with the two black marks visible to the endoscope) is also the spline that has been rendered radiopaque on its proximal half by the first radiopaque marker 1200 and is as such visible in the fluoroscopic image. As also mentioned, the location and longitudinal extent of the electrodes on each spline are preferably identified with a marker that is visible under endoscope and thus, can be in the form of a light colored (e.g., white) band of paint that is applied to the inner surface of the spline so as to be seen on the endoscope. To then specially identify the one reference spline/reference electrode, in this case the spline #1, another visual marker, in this case the two black squares, is applied to the inner surface of the spline #1. This allows the surgeon to easily and quickly determine the location of the spline #1 in the endoscopic image.

FIGS. 41A, 42A, 43A, 44A, 45A show the balloon 1005 in profile for various rotational orientations of the balloon 1005 as the balloon 1005 would appear in a fluoroscopic image. It is standard convention to orient the fluoroscopic image so that the top if the image is toward the patient's head and the images of FIGS. 41A, 42A, 43A, 44A, 45A are so oriented. In the fluoroscopy images of FIGS. 41A, 42A, 43A, 44A, 45A, the distal end of the ballon 1005 is to the right and the proximal end of the balloon 1005 is to the left.

As described herein, the endoscopic images in FIGS. 41B and 41C correspond to the fluoroscopy image in FIG. 41A and similarly, the endoscopic images in FIGS. 42B and 42C correspond to the fluoroscopy image in FIG. 42A; the endoscopic images in FIGS. 43B and 43C correspond to the fluoroscopy image in FIG. 43A; the endoscopic images in FIGS. 44B and 44C correspond to the fluoroscopy image in FIG. 44A; and the endoscopic images in FIGS. 45B and 45C correspond to the fluoroscopy image in FIG. 45A.

The images of FIGS. 41B, 42B, 43B, 44B, 45B each shows the initial endoscopic view where the endoscope is looking out through the transparent balloon 1005 toward the patient's pulmonary vein antrum and where the orientation of the vein anatomy is initially random because the rotational orientation of the endoscope in the catheter and the rotational orientation of the balloon catheter in the patient's anatomy may both be random. Note that in the endoscopic images we also see the inner surfaces of the twelve splines which appear as twelve radial lines (in a spoke like pattern and visible due to the applied white paint on the inner surfaces of the splines). We see also spline #1 which has been marked with the two black marks and we also see the central nitinol shaft of the catheter appearing as a pie-shaped member (identified by cross hatching) between two of the splines. In some embodiments of the catheter the central shaft may occupy enough of the endoscopic image that one or two of the splines are obscured by the central shaft. In this case, the functionality and features described here still function perfectly well since only visualization of spline #1 is essential to the image processing steps. During manufacture of the balloon catheter, it is a straightforward to construct the balloon catheter such that the rotational position of spline #1 relative to the position of the endoscope is such that spline #1 is not obscured from view during endoscopic imaging.

The endoscopic images of FIGS. 41C, 42C, 43C, 44C, 45C shows the final (altered/manipulated) orientation of the endoscopic image such that the top of the image corresponds to the direction of the patient's head. Referring to these images, the procedure to reorient the endoscopic image to the final anatomically correct orientation is as follows: In FIG. 41A, one observes the location of the radiopaque splines (identified by the radiopaque markers 1200, 1202). Here, the goal is to first determine the rotational orientation of spline #1 (the spline whose proximal half has been rendered radiopaque by marker 1200) relative to the patient's anatomy. In FIG. 41A, one sees that spline #1 is on the uppermost part of the balloon 1005. Since, as stated earlier, the top of the fluoroscopic image is toward the patient's head it follows that spline #1 is closest to the patient's head. In other words, in FIG. 41A, the spline #1 is in fact directly facing toward the patient's head (since it is located at the topmost location of the balloon in the fluoroscopy image). Now turning to FIG. 41B, one can see spline #1 (identified by the two black marks) in the live endoscopic view. Initially spline #1 happens to be randomly oriented to the left in the endoscopic image. Since the fluoroscopic image indicates that spline #1 is in reality located directly toward the patient's head, the reorientation guidance is that one must rotate the endoscopic image of FIG. 41B to the orientation shown in the image in FIG. 41C where spline #1 (with the two black marks) is uppermost (at the 12 o'clock position) in the final, re-oriented endoscopic image. The means for the user to rotate the endoscopic image could be via electronically rotating the image on the display screen using touch screen controls or other mechanical control buttons or sliders to set the degree of rotation desired. Alternatively, in systems where the endoscope consists of an optical fiber bundle removably connected to a camera, the image may be rotated by rotating the optical fiber bundle connector relative to the camera. This action has the effect of rotating the endoscopic image on the display screen. The user simply rotates the connector until the endoscopic image appears as desired with spline #1 uppermost position as shown in FIG. 41C. Alternatively, the rotation can be done by a processor of a computer in that the live endoscopic image itself is rotated on the screen by a processor as described herein.

Now turning to FIGS. 42A-42C and 43A to 43C. In both FIGS. 42A-42C and 43A to 43C, Spline #1 is not in the uppermost position but is rotated 45 degrees from the uppermost position. In FIGS. 42A-42C, the balloon 1005 is rotated so that spline #1 is rotated 45 degrees towards the viewer of the fluoroscopic image (a clockwise rotation of 45 degrees when looking axially along the catheter from the proximal end of the balloon to the distal end) and, conversely, in FIGS. 43A-43C, the balloon 1005 is rotated so that spline #1 is rotated 45 degrees away from the viewer (a counterclockwise rotation of 45 degrees). Here one will note that if only spline #1 was rendered radiopaque (as by marker 1200), one would not be able to tell the difference between a 45-degree clockwise rotation and a 45-degree clockwise rotation (See, discussion of FIG. 39A). This is obvious from that fact that the fluoroscopic appearance of spline #1, with its proximal portion made radiopaque, is identical for a 45-degree clockwise rotation (FIG. 42A) or a 45 counterclockwise rotation (FIG. 43A). But, since we have also rendered the distal half of a second spline radiopaque as by marker 1202, and this second spline is known to be oriented 90 degrees clockwise from spline #1, the two different 45-degree rotations shown in FIG. 42A and FIG. 43A present themselves as unique, unambiguous configurations of the radiopaque splines.

As before, when we were considering FIGS. 41A-41C, the initial endoscopic image (FIG. 41B) has a random orientation. To adjust the endoscopic image to its proper anatomical orientation (i.e., FIG. 41C), the user rotates the image so that Spline #1 with the two black marks is 45 degrees clockwise from the 12 o'clock position in the case shown in FIG. 42C. Similarly, one rotates the endoscopic image 45 degrees counterclockwise from the 12 o'clock position in the case shown in FIG. 43C.

Continuing to FIGS. 44A-C and FIGS. 45A-45C, these rows show balloon orientations with spline #1 rotated 90 degrees from the position of FIGS. 41A-41C; with FIGS. 44A-44C showing a 90-degree clockwise rotation and FIGS. 45A-45C showing a 90-degree counterclockwise rotation. (again, one defines clockwise rotation as the rotational direction observed when looking from the proximal end of the balloon toward the distal end). Again, one notes that if one had only rendered Spline #1 radiopaque (as by marker 1200), one would not be able to distinguish a 90-degree clockwise rotation as we see in FIGS. 44A-44C from a counterclockwise rotation of FIGS. 45A-45C. Again, the initial orientations of the endoscopic images are random for reasons explained already and again, to obtain an anatomically correctly oriented endoscopic image, one rotates the endoscopic image so that spline #1 with the two black marks is in the 3 o'clock position for FIGS. 44A-44C and in the 9 o'clock position for FIGS. 45A-45C.

Accordingly, the present disclose teaches rendering radiopaque at least part of at least two splines and additionally an endoscopically visible mark on at least one of the radiopaque splines such that first, the rotational orientation of the balloon relative to the anatomy may be unambiguously determined using fluoroscopic imaging and second, the live endoscopic image may be rotated by the user so that the direction toward the patient's head is uppermost in the rotated (altered) final endoscopic image that is displayed to the user. As an additional alternative is possible for both the initial endoscopic image and the fluoroscopic image to be analyzed by computer rather than by the human user and the rotation of the endoscopic performed automatically electronically by the computer. In this case, image recognition software and AI can be used so that the fluoroscopy image (e.g., FIG. 41A) and the initial endoscopic image (e.g., FIG. 41B) are analyzed by software and then the initial endoscopic image is manipulated (e.g., rotated) to the target final endoscopic image orientation which is displayed on a screen or the like, such as display 14.

It will be understood that the endoscopic images represent a live video feed and thus, the reoriented endoscopic image represents a reoriented live video feed that can be viewed in real-time on display 14.

Thus, by examining the orientation of the radiopaque markers under fluoroscopy, the user can determine the desired orientation of the endoscopic image(s). Once the desired orientation of the is determined, images in the endoscopic video stream can be re-oriented (e.g., rotated) to ensure the relative positions of aspects, such as the superior aspect of the vein, can be adjusted appropriately. For example, the images can be rotated so that the superior aspect of the vein is oriented at the 12 o'clock position, while the inferior aspect is oriented at the 6 o'clock position, the anterior aspect is provided at the 3 o'clock position and the posterior aspect is provided at the 9 o'clock position.

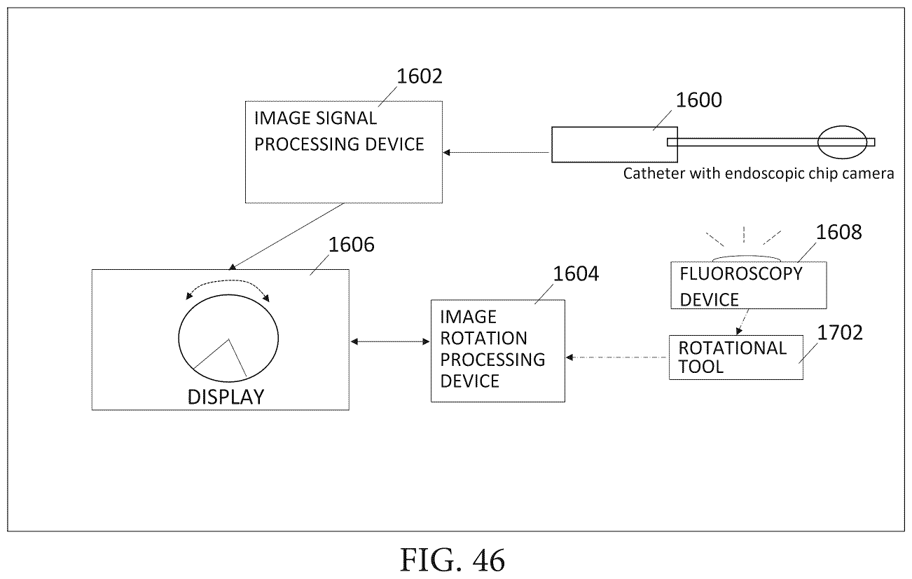

Referring now to FIG. 46, a system diagram is provided that shows an example arrangement that includes the catheter with one or more endoscopic chip camera(s) 1600, image signal processing device 1602, image rotation processing device 1604, and display device 1606. Further, the example devices shown in FIG. 46 include fluoroscopy device 1608 and rotational tool 1702. Although the example in FIG. 46 shows the image signal processing device 1602 and image rotation processing device 1604 as separate devices, it is recognized that devices 1602 and 1604 can be configured in one single processing device.

The devices shown in FIG. 46 can provide for altering the orientation (e.g., rotating) of images from in the endoscopic video stream once the desired rotational orientation has been determined. Solid or dashed line connections between the respective devices can represent transmissions using any known arrangement or technique for sending and receiving information to and from devices.

In the example system shown in FIG. 46, the image signal processing device 1602 can interface with the catheter with endoscopic chip camera(s) 1600, including to convert signals from the chip camera(s) into a standard video signal, such as an analog NTSC signal or HDMI signal. Alternatively, the image signal processing device 1602 can convert signals received from the chip camera(s) into a video stream capable of being transferred to the image rotation processing device 1604 which can be a computer, such as via a USB or other suitable interface. The image rotation processing device 1604 can operate to manipulate images within the video stream to display the images in any rotation on the display device 1606, including as selected by the user.

In one or more embodiments, a graphical user interface can be included with controls for the user to define a desired rotation. For example, a user can cause a clockwise or counterclockwise rotation by tapping a touchscreen device, clicking a mouse or other selector device, turning a virtual or physical knob, pressing a virtual or physical button, or by selecting some other suitable interface control. Further, one or more parameters can be set that defines various properties, such as the direction of rotation and/or predefined increments (or custom amounts) of rotation that are suitable for a respective user. Other implementations are similarly supported and envisioned, such as to provide an interface by which a user makes a selection using a touchscreen, mouse, or other suitable interface gestures (e.g., dragging, swiping, pinching/zooming, or the like), which cause a processor to rotate an image by a particular amount and in a respective direction.

The rotation tool 1702 thus allows the rotation of the initial endoscopic image shown in FIGS. 41B, 42B, 43B, 44B and 45B to the reoriented endoscopic image shown in FIGS. 41C, 42C, 43C, 44C and 45C. In one embodiment, the rotational tool 1702 can include a rotatable graphical control that, when selected, can be used by the user to rotate the orientation of the radiopaque markers to match the orientation of radiopaque markers 1200, 1202 shown in the fluoroscopic image. As also mentioned, this rotation can occur in an automated manner due to software.

More specifically, in one or more implementations, orientation of an endoscopic image is altered during a surgical procedure. For example, at least one computing devices accesses a first image, which can be a fluoroscopic image, of at least part of a catheter during the surgical procedure. The catheter includes a first spline and a second spline of an electrode basket, wherein the catheter in the first image is in a respective orientation, and further wherein the first spline has a first marking and the second spline has a second marking. Further, during the surgical procedure, at least one computing device accesses an endoscopic image in an orientation of at least part of the catheter, wherein the catheter in the endoscopic image shows a third marking formed along a rear face of the first spline. The computing device provides a graphical user interface during the surgical procedure, which displays the endoscopic image. The at least one computing device alters the orientation of the endoscopic image as a function of the first marking, the second marking, and the third marking, during the surgical procedure.

In one or more implementations, altering the orientation of the endoscopic image further includes determining, by the at least one computing device, the orientation of the first spline in the first image as a function of the first marking and the second marking. The computing device alters the orientation of the endoscopic image until the third marking is positioned relative to the determined orientation of the catheter in the first image. Altering the orientation of the endoscopic image can occur automatically by the at least one computing device, for example, processing at least the first image to determine the orientation (e.g., the location of) the first marker and second marker.

Further, the graphical user interface can be configured to provide an alert, such as a change of color, a flash, or graphical element (e.g., a needle) or other feature to represent when the orientation of the endoscopic image is relative to the first marker and second marker.

### Contact Detection System

Applicant's previous application U.S. Ser. No. 18/622,232, which is hereby expressly incorporated by reference in its entirety, discloses different local impedance detectors that can be utilized in the catheters disclosed herein.

In one embodiment, a system for delivering energy to tissue of a patient is provided and comprises:
a catheter having at least one electrode;
at least one impedance sensor correlated to the at least one electrode; and
an impedance indicator that indicates sensed impedance by the at least one impedance sensor.

The impedance indicator can comprise a visual image on a display based on the sensed impedance. The impedance indicator can be the sum of all electrode inputs and displayed as a vector. The vector can be sent and displayed on a mapping system as a 3D vector on a moving catheter graphic.

The algorithm to determine a summative impedance vector/direction is determined by principal component analysis or similar statistical analysis of time-varying data. The magnitude of the display can be integrated into haptic feedback within the catheter.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

What is claimed is:

1. A balloon catheter comprising:
a handle;
an outer catheter shaft coupled to the handle;
an inflatable balloon coupled at a first end to the outer catheter shaft;
an axially translatable nose tip to which a second end of the inflatable balloon is coupled;
an electrode basket surrounding the balloon and being coupled to the outer catheter shaft and to the nose tip, the electrode basket having a plurality of first splines and a plurality of second splines, wherein the plurality of first splines and the plurality of second splines are configured to deploy under inflation of the inflatable balloon and wherein at least some first splines of the plurality of first splines include at least one electrode;
an actuator disposed within the handle for axially translating the nose tip in a longitudinal direction to facilitate the electrode basket moving to a collapsed state when the balloon is deflated and to an expanded state when the balloon is inflated, the actuator moving axially within the handle between an extended position and a retracted position; and
a lock mechanism for locking the actuator in the extended position.

2. The balloon catheter of claim 1, wherein the actuator is spring biased and assumes the extended position as an at rest position and assumes the retracted position when the balloon is inflated and a force of the balloon on the plurality of first and second splines overcomes a spring force of the spring.

3. The balloon catheter of claim 1, wherein the handle has an elongated opening formed therein in which the actuator is disposed, the elongated opening having opposite distal and proximal ends, wherein the spring is disposed between a proximal edge of the actuator and the proximal end of the elongated opening.

4. The balloon catheter of claim 1, wherein the handle has an elongated opening formed therein in which the actuator is disposed and the elongated opening has opposite distal and proximal ends, and wherein the actuator comprises an elongated actuator shaft that is coupled to the nose tip and a slider contained at least partially within the elongated opening and a rotatable knob that is carried by the slider, the actuator shaft being coupled to the knob such that axial movement and rotation of the knob is imparted to the actuator shaft.

5. The balloon catheter of claim 1, wherein the lock mechanism includes a lock plate that is fixedly attached to the handle and a lock shaft that is coupled to the actuator, wherein the lock plate has a first locking part and the lock shaft has a second locking part that selectively engages the first locking part to lock the actuator in the extended position.

6. The balloon catheter of claim 5, further including a torque translation cap that is rotatably coupled to the lock plate and a torsion spring that is coupled between the lock plate and the torque translation cap, the torque translation cap applying a torque force to the lock shaft to promote the engagement of the second locking part to the first locking part.

7. The balloon catheter of claim 6, wherein the lock plate has a through hole through which the lock shaft passes at least when the actuator is in the retraced position and the torsion spring has a proximal end attached to the lock plate and a distal end attached to the torque translation cap.

8. The balloon catheter of claim 5, wherein the second locking part comprises a plurality of rounded teeth formed along at least one fin of the lock shaft and the first locking part comprises an undulating surface formed along the lock plate, wherein the first and second locking parts engage one another when peaks of the plurality of rounded teeth seat within valleys of the undulating surface, whereby the lock shaft is held in place.

9. The balloon catheter of claim 8, wherein the lock shaft has two fins that are located 180 degrees apart, with the plurality of rounded teeth being formed along a length of each of the two fins and the first locking part comprises two undulating surfaces spaced apart from one another.

10. The balloon catheter of claim 5, wherein the handle has an elongated opening formed therein in which the actuator is disposed and the elongated opening has opposite distal and proximal ends, and wherein the actuator comprises an elongated actuator shaft that is coupled to the nose tip and a slider contained at least partially within the elongated opening and a rotatable knob that is carried by the slider, the actuator shaft being coupled to the knob such that axial movement and rotation of the knob is imparted to the actuator shaft, the lock shaft being fixedly coupled to the knob.

11. The balloon catheter of claim 6, wherein the first and second locking parts permit axial movement of the lock shaft in a ratcheting manner, and the applied torque force rotates the lock shaft in a counter-clockwise direction when peaks of the plurality of rounded teeth align with valleys of the undulating surface, thereby pulling the first and second locking parts together.

12. The balloon catheter of claim 11, wherein the second locking part comprises a plurality of sharp teeth formed along at least one fin of the lock shaft and the first locking part comprises a toothed surface formed along the lock plate, wherein the first and second locking parts engage one another when peaks of the plurality of sharp teeth seat within valleys of the toothed surface, whereby the lock shaft is held in place, the actuator being configured such that rotation thereof causes disengagement of the sharp teeth from the toothed surface to allow axial movement of the lock shaft.

13. The balloon catheter of claim 6, wherein the lock plate has a through hole through which the lock shaft passes at least when the actuator is in the retraced position, wherein the second locking part comprises at least one fin formed longitudinally along the lock shaft and the first locking part comprises a locking space that receives the at least one fin when the at least one fin clears the through hole and due to the torque force being applied to the lock shaft which automatically rotates the at least one fin into the locking space, wherein reception of the at least one fin within the locking space prevents proximal movement of the lock shaft and locks the actuator in the extended position.

14. The balloon catheter of claim 13, wherein the at least one fin comprises two fins that are formed 180 degrees apart from one another and the through hole includes two keyed slots through which the fins pass when the actuator is not in the extended position and the lock plate includes two locking spaces for receiving the two fins.

15. A balloon catheter comprising:
an outer catheter shaft;
an inflatable balloon coupled at a first end to the outer catheter shaft;
a translatable nose tip to which a second end of the inflatable balloon is coupled;
an electrode basket having a plurality of first splines that surround the balloon;
an actuator for axially translating the nose tip, the electrode basket being coupled at a first end to the outer catheter shaft and at a second end to the nose tip, wherein one or more of the first splines support one or more electrodes; the actuator being configured to permit the electrode basket to assume a collapsed state when the balloon is deflated and when the nose tip is translated to a distal position;
wherein the plurality of first splines are configured to deploy and radially expand under inflation of the inflatable balloon;
wherein the outer catheter shaft surrounds a plurality of inner lumen structures including an endoscope lumen in which an endoscope passes and a first lumen that carries an axially translatable actuator shaft that is coupled to the nose tip at a first end such that axial movement of the actuator shaft imparts axial translation of the nose tip and permits the electrode basket to move between the collapsed state and a deployed state when the balloon is inflated; and
wherein positions of the tube lumen and the endoscope lumen are fixed within the outer catheter shaft at distal end of the outer catheter shaft.

16. The balloon catheter of claim 15, wherein a distance between the tube lumen and the endoscope lumen within the outer catheter shaft is maximized.

17. The balloon catheter of claim 15, further including an internal mass at the distal end of the outer catheter shaft that fixes the positions of the tube lumen and the endoscope lumen.

18. The balloon catheter of claim 17, wherein the internal mass comprises a potting compound surrounding the inner lumen structures.

19. The balloon catheter of claim 17, wherein the internal mass comprises an endcap through which the first lumen and endoscope lumen pass.

20. The balloon catheter of claim 15, wherein the translatable actuator shaft has a spiral (helical) cut section that acts as a spring when a force is applied to the translatable actuator shaft.

21. The balloon catheter of claim 20, wherein the spiral cut section is located proximal to and outside of the inflatable balloon.

22. The balloon catheter of claim 20, wherein the spiral cut section is disposed between the inflatable balloon and a handle of the catheter.

23. The balloon catheter of claim 15, further including a lock mechanism for locking a position of the translatable tube and thereby lock a position of the electrode basket.

24. The balloon catheter of claim 23, wherein the lock mechanism is configured to lock the translatable actuator shaft in a forward position resulting in the electrode basket being locked in the collapsed state.

25. The balloon catheter of claim 23, wherein the lock mechanism engages an axially movable knob that is contained within the handle, the translatable actuator shaft being coupled to the knob such that axial movement of the knob is translated into axial movement of the translatable actuator shaft.
